SEQUENCE LISTING (51 sequences)

SEQ ID NO: 1 - protein, 532 aa, Saccharomyces cerevisiae
MLLQAFLFLLAGFAAKISASMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 2 - protein, 602 aa, Saccharomyces cerevisiae
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 3 - protein, 513 aa, Saccharomyces cerevisiae
SMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 4 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDAKEAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 5 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDAKWHLYFQYNPNDTVWGLPLFWGHATSDDLTHWQDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGILDQNILELYFNDGDVVSTNTYFMTTGNALGSVNMTTGVDNLFYIDKFQVREVK

SEQ ID NO: 6 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKAVFADLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFEVREVK

SEQ ID NO: 7 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAALSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKNVFPDLSLWFKGLEDPEEYLRMGFEASASSFFLDRGNSPVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 8 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWTLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETSLINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSINMTTGVDNLFYIDKFQVREVK

SEQ ID NO: 9 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDGKWHLYFQYNPNDTVWGLPLYWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 10 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTDPWRSSMSLVRKFSLNTEYQANPETELINLKAEPILNISNAGPWSRFATNTTLTKANSYNVDLSNSTGTLEFELVYAVNTTQTISKNVFPDLSLWFKGLEDPEEYLRMGFEASASSFFLDRGNSKVKFVRENPYFTNRMSVNNQPFKSENDLSYYKVYGLLDQNILELYFNDGDVVSTNTYFMTTGNALGSVNMTTGVDNLFYIDKFQVREVK

SEQ ID NO: 11 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWQYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMSTGNALGSINMTTGVDNLFYIDKFQVREVK

SEQ ID NO: 12 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKEAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTSQTISKNVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 13 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDAKWHLYFQYNPNDTVWGQPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEASASSFFLDRGNSPVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 14 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDAKWHLYFQYNPNDTVWGTPLFWGHATSDDLTHWEDEPIAIAPKRNDSGAYSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISAAGPWSRFATNTTLTKANSYNVDLSNSTGTLEFELVYAVNTTQTISKSVFADLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 15 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDAKDAKWHLYFQYNPNDTVWGTPLFWGHATSDDLTHWEDEPIAIAPKRNDSGAFSGSMVIDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGILDQNILELYFNDGDVVSTNTYFMTTGNALGSVNMTTGVDNLFYIDKFQVREVK

SEQ ID NO: 16 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWQYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKDENDLSYYKVYGLLDQNILELYFNDGDVVSTNTYFMTTGNALGSVNMTTGVDKLFYIDKFQVREVK

SEQ ID NO: 17 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDAKWHLYFQYNPNDTVWGLPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTSVDNLFYIDKIQVREVK

SEQ ID NO: 18 - protein, 602 aa, synthetic construct
MRFPSIFTAVLFAASSALAAPVNTTTEDETAQIPAEAVIGYSDLEGDFDVAVLPFSNSTNNGLLFINTTIASIAAKEEGVSLEKREAEASMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWQYSAFVPTNPWRSSMSLVRKFSLNTEYQANPETELINLKAEPILNISNAGPWSRFATNTTLTKANSYNVDLSNSTGTFEFELVYAVNTTQTISKSVFPDLSLWFKGLEDPEEYLRMGFEASASSFFLDRGNSKVKFVRENPYFTNRMSVNNQPFKSENDLSYYKVYGLLDQNILELYFNDGDVVSTNTYFMTTGNALGSVNMTTGVDNLFYIDKFQVREVK

SEQ ID NO: 19 - protein, 513 aa, synthetic construct
SMTNETSDRPLVHFTPNKGWMNDPNGLWYDAKEAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 20 - protein, 513 aa, synthetic construct
SMTNETSDRPLVHFTPNKGWMNDPNGLWYDEKDAKWHLYFQYNPNDTVWGLPLFWGHATSDDLTHWQDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGILDQNILELYFNDGDVVSTNTYFMTTGNALGSVNMTTGVDNLFYIDKFQVREVK

SEQ ID NO: 21 - protein, 513 aa, synthetic construct
SMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKAVFADLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFEVREVK

SEQ ID NO: 22 - protein, 513 aa, synthetic construct
SMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAALSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKNVFPDLSLWFKGLEDPEEYLRMGFEASASSFFLDRGNSPVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 23 - protein, 513 aa, synthetic construct
SMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWTLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETSLINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSINMTTGVDNLFYIDKFQVREVK

SEQ ID NO: 24 - protein, 513 aa, synthetic construct
SMTNETSDRPLVHFTPNKGWMNDPNGLWYDEKDGKWHLYFQYNPNDTVWGLPLYWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 25 - protein, 513 aa, synthetic construct
SMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTDPWRSSMSLVRKFSLNTEYQANPETELINLKAEPILNISNAGPWSRFATNTTLTKANSYNVDLSNSTGTLEFELVYAVNTTQTISKNVFPDLSLWFKGLEDPEEYLRMGFEASASSFFLDRGNSKVKFVRENPYFTNRMSVNNQPFKSENDLSYYKVYGLLDQNILELYFNDGDVVSTNTYFMTTGNALGSVNMTTGVDNLFYIDKFQVREVK

SEQ ID NO: 26 - protein, 513 aa, synthetic construct
SMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWQYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMSTGNALGSINMTTGVDNLFYIDKFQVREVK

SEQ ID NO: 27 - protein, 513 aa, synthetic construct
SMTNETSDRPLVHFTPNKGWMNDPNGLWYDEKEAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTSQTISKNVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 28 - protein, 513 aa, synthetic construct
SMTNETSDRPLVHFTPNKGWMNDPNGLWYDEKDAKWHLYFQYNPNDTVWGQPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEASASSFFLDRGNSPVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 29 - protein, 513 aa, synthetic construct
SMTNETSDRPLVHFTPNKGWMNDPNGLWYDEKDAKWHLYFQYNPNDTVWGTPLFWGHATSDDLTHWEDEPIAIAPKRNDSGAYSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISAAGPWSRFATNTTLTKANSYNVDLSNSTGTLEFELVYAVNTTQTISKSVFADLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 30 - protein, 513 aa, synthetic construct
SMTNETSDRPLVHFTPNKGWMNDPNGLWYDAKDAKWHLYFQYNPNDTVWGTPLFWGHATSDDLTHWEDEPIAIAPKRNDSGAFSGSMVIDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGILDQNILELYFNDGDVVSTNTYFMTTGNALGSVNMTTGVDNLFYIDKFQVREVK

SEQ ID NO: 31 - protein, 513 aa, synthetic construct
SMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWQYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKDENDLSYYKVYGLLDQNILELYFNDGDVVSTNTYFMTTGNALGSVNMTTGVDKLFYIDKFQVREVK

SEQ ID NO: 32 - protein, 513 aa, synthetic construct
SMTNETSDRPLVHFTPNKGWMNDPNGLWYDEKDAKWHLYFQYNPNDTVWGLPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFPDLSLWFKGLEDPEEYLRMGFEASASSFFLDRGNSKVKFVKENPYFTNRMSVNNQPFKSENDLSYYKVYGLLDQNILELYFNDGDVVSTNTYFMTTGNALGSVNMTTSVDNLFYIDKIQVREVK

SEQ ID NO: 33 - protein, 513 aa, synthetic construct
SMTNETSDRPLVHFTPNKGWMNDPNGLWYDEKDAKWHLYFQYNPNDTVWGTPLFWGHATSDDLTHWEDEPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWQYSAFVPTNPWRSSMSLVRKFSLNTEYQANPETELINLKAEPILNISNAGPWSRFATNTTLTKANSYNVDLSNSTGTFEFELVYAVNTTQTISKSVFPDLSLWFKGLEDPEEYLRMGFEASASSFFLDRGNSKVKFVRENPYFTNRMSVNNQPFKSENDLSYYKVYGLLDQNILELYFNDGDVVSTNTYFMTTGNALGSVNMTTGVDNLFYIDKFQVREVK

SEQ ID NO: 34 - protein, 89 aa, unidentified
  source 1..89: note = FAKS signal sequence
MRFPSIFTAVLFAASSALAAPVNTTTEDETAQIPAEAVIGYSDLEGDFDVAVLPFSNSTNNGLLFINTTIASIAAKEEGVSLEKREAEA

SEQ ID NO: 35 - DNA, 1599 bp, Saccharomyces cerevisiae
  source 1..1599: mol_type = genomic DNA
atgcttttgcaagctttccttttccttttggctggttttgcagccaaaatatctgcatcaatgacaaacgaaactagcgatagacctttggtccacttcacacccaacaagggctggatgaatgacccaaatgggttgtggtacgatgaaaaagatgccaaatggcatctgtactttcaatacaacccaaatgacaccgtatggggtacgccattgttttggggccatgctacttccgatgatttgactcattgggaagatgaacccattgctatcgctcccaagcgtaacgattcaggtgctttctctggctccatggtggttgattacaacaacacgagtgggtttttcaatgatactattgatccaagacaaagatgcgttgcgatttggacttataacactcctgaaagtgaagagcaatacattagctattctcttgatggtggttacacttttactgaataccaaaagaaccctgttttagctgccaactccactcaattcagagatccaaaggtgttctggtatgaaccttctcaaaaatggattatgacggctgccaaatcacaagactacaaaattgaaatttactcctctgatgacttgaagtcctggaagctagaatctgcatttgctaatgaaggtttcttaggctaccaatatgaatgtccaggtttgattgaagtcccaactgagcaagatccttccaaatcctattgggtcatgtttatttctatcaatccaggtgcacctgctggcggttccttcaaccaatattttgttggatccttcaatggtactcattttgaagcgtttgacaatcaatctagagtggtagattttggtaaggactactatgccttgcaaactttcttcaacacagacccaacgtacggttcagcattaggtattgcctgggcttcaaactgggagtacagtgcctttgtcccaactaacccatggagatcatccatgtctttggtccgcaagttttctttgaacactgaatatcaagctaatccagagactgaattgatcaatttgaaagccgaaccaatattgaacattagtaatgctggtccctggtctcgttttgctactaacacaactctaactaaggccaattcttacaatgtcgatttgagcaactcgactggtaccctagagtttgagttggtttacgctgttaacaccacacaaaccatatccaaatccgtctttcccgacttatcactttggttcaagggtttagaagatcctgaagaatatttaagaatgggttttgaagccagtgcttcttccttctttttggaccgtggtaactctaaggtcaagtttgtcaaggagaacccatatttcacaaacagaatgtctgtcaacaaccaaccattcaagtctgagaacgacctaagttactataaagtgtacggcctactggatcaaaacatcttggaattgtacttcaacgatggagatgtggtttctacaaatacctacttcatgaccaccggtaacgctctaggatctgtgaacatgaccactggtgtcgataatttgttctacattgacaagttccaagtaagggaagtaaaatag

SEQ ID NO: 36 - protein, 532 aa, Saccharomyces cerevisiae
MLLQAFLFLLAGFAAKISASMTNETSDRPLVHFTPNKGWMNDPNGLWYDEKDAKWHLYFQYNPNDTVWGTPLFWGHATSDDLTNWEDQPIAIAPKRNDSGAFSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISNAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 37 - protein, 589 aa, Saccharomyces cerevisiae
MTISSAHPETEPKWWKEATFYQIYPASFKDSNDDGWGDMKGIASKLEYIKELGADAIWISPFYDSPQDDMGYDIANYEKVWPTYGTNEDCFALIEKTHKLGMKFITDLVINHCSSEHEWFKESRSSKTNPKRDWFFWRPPKGYDAEGKPIPPNNWKSYFGGSAWTFDEKTQEFYLRLFCSTQPDLNWENEDCRKAIYESAVGYWLDHGVDGFRIDVGSLYSKVVGLPDAPVVDKNSTWQSSDPYTLNGPRIHEFHQEMNQFIRNRVKDGREIMTVGEMQHASDETKRLYTSASRHELSELFNFSHTDVGTSPLFRYNLVPFELKDWKIALAELFRYINGTDCWSTIYLENHDQPRSITRFGDDSPKNRVISGKLLSVLLSALTGTLYVYQGQELGQINFKNWPVEKYEDVEIRNNYNAIKEEHGENSEEMKKFLEAIALISRDHARTPMQWSREEPNAGFSGPSAKPWFYLNDSFREGINVEDEIKDPNSVLNFWKEALKFRKAHKDITVYGYDFEFIDLDNKKLFSFTKKYNNKTLFAALNFSSDATDFKIPNDDSSFKLEFGNYPKKEVDASSRTLKPWEGRIYISE

SEQ ID NO: 38 - protein, 589 aa, Saccharomyces cerevisiae
MTISSAHPETEPKWWKEATIYQIYPASFKDSNNDGWGDMKGIASKLEYIKELGADAIWISPFYDSPQDDMGYDIANYEKVWPTYGTNEDCFALIEKTHKLGMKFITDLVINHCSSEHEWFKESRSSKTNPKRDWFFWRPPKGYDAEGKPIPPNNWRSYFGGSAWTFDEKTQEFYLRLFCSTQPDLNWENEDCRKAIYESAVGYWLDHGVDGFRIDVGSLYSKVAGLPDAPVIDENSKWQPSDPFTMNGPRIHEFHQEMNKFIRNRVKDGREIMTVGEMQHATDETKRLYTSASRHELSELFNFSHTDVGTSPKFRQNLIPYELKDWKVALAELFRYVNGTDCWSTIYLENHDQPRSITRFGDDSPKNRVISGKLLSVLLVSLSGTLYVYQGQELGEINFKNWPIEKYEDVEVRNNYDAIKEEHGENSKEMKRFLEAIALISRDHARTPMQWSREEPNAGFSGPNAKPWFYLNESFREGINAEDESKDPNSVLNFWKEALRFRKAHKDITVYGYDFEFIDLDNKKLFSFTKKYDNKTLFAALNFSSDSIDFTIPNNSSSFKLEFGNYPRSEVDASSRTLKPWEGRIYISE

SEQ ID NO: 39 - protein, 589 aa, Saccharomyces cerevisiae
MTISSAHPETEPKWWKEATIYQIYPASFKDSNNDGWGDMKGIASKLEYIKELGTDAIWISPFYDSPQDDMGYDIANYEKVWPTYGTNEDCFALIEKTHKLGMKFITDLVINHCSSEHEWFKESRSSKTNPKRDWFFWRPPKGYDAEGKPIPPNNWRSYFGGSAWTFDEKTQEFYLRLFCSTQPDLNWENEDCRKAIYESAVGYWLDHGVDGFRIDVGSLYSKVAGLPDAPVIDENSKWQLSDPFTMNGPRIHEFHQEMNKFIRNRVKDGREIMTVGEMRHATDETKRLYTSASRHELSELFNFSHTDVGTSPKFRQNLIPYELKDWKVALAELFRYVNGTDCWSTIYLENHDQPRSITRFGDDSPKNRVISGKLLSVLLVSLSGTLYVYQGQELGEINFKNWPIEKYEDVEVRNNYDAIKEEHGENSKEMKRFLEAIALISRDHARTPMQWSREEPNAGFSGPNAKPWFYLNESFREGINAEDESKDPNSVLNFWKEALRFRKAHKDITVYGYDFEFIDLDNKKLFSFTKKYDNKTLFAALNFSSDSIDFTIPNNSSSFKLEFGNYPRSEVDASSRTLKPWEGRIYISE

SEQ ID NO: 40 - protein, 589 aa, Saccharomyces cerevisiae; identical to SEQ ID NO: 39

SEQ ID NO: 41 - protein, 581 aa, Saccharomyces cerevisiae
MTIIHNPKWWKEATVYQIYPASFKDSNNDGWGDLAGITSKLDYVKELGVDAIWVCPFYDSPQEDMGYDIANYEKVWPRYGTNEDCFQMIEEAHKRGIKVIVDLVINHCSEEHEWFKESRSSKANPKRDWFFWRPPKGYDEKGNPIPPNNWRSFFGGSAWRYDEKTGEFFLHVFALGQPDFNWENEECRKAIYDSSVGYWLRHNVDGFRIDVGSMYSKVEGLPDAPITDPTVPYQKGTEFFINGPRIHEYHKEMHNYMLSQVPEGKEIMTVGEVGIGNEDDFRVYTSAKEGELNMMFNFKHTSVGENPKCKYELIPFTLKDFKLALAESFLFIENTDCWSTIYLENHDQPRSVSRFGSDSPKWREISSKMLATLIISLTGTVFIYQGQELGMPNFKNRKIEQIKCVEGTGTYAAIKRDYGEDSEKMKKFFEALALISRDHGRTPFPWSADEPSAGFSKDAKPWIDMNESFRDGINAEAELKDKNSVFFFWKKALQVRKEHKDILVYGHNFQFIDLDNDKLFMFTKDTDNKKMFAVFNFSSDNTDFSVPDNEASYTMFFGNYANSNGDSRTLQPWEGRLYLLK

SEQ ID NO: 42 - DNA, 1770 bp, Saccharomyces cerevisiae
  source 1..1770: mol_type = genomic DNA
atgactatttcttctgcacatccagagacagaaccaaagtggtggaaagaggccacgttctatcaaatttacccagcaagtttcaaagactctaatgacgatggctggggtgacatgaaagggattgcctccaagctggagtacatcaaagagcttggtgccgatgccatttggatctcaccattctacgactcgccacaagatgatatgggttacgatattgccaactacgaaaaggtctggccaacatatggtacgaatgaagactgctttgccttgatcgaaaagacacataagcttggtatgaaatttatcaccgacttggtcatcaatcactgttccagcgaacatgaatggttcaaagagagcagatcctcgaagactaatccaaagcgtgactggttcttctggagacctcctaaaggttatgacgccgaaggcaagccaattcctccaaacaattggaaatcctattttggtggttccgcatggaccttcgatgaaaagacacaagaattctacttgcgtttgttttgctccactcaacctgatttgaattgggagaatgaagactgtagaaaggcaatctacgaaagtgccgttggatactggttagaccatggtgtagacggctttagaattgatgtcggaagtttgtactccaaagttgtaggtttaccagatgctcctgttgttgacaaaaactcgacttggcaatccagtgatccatacacattgaatggaccacgtattcacgagttccatcaagaaatgaatcaattcatcagaaacagagtgaaggatggcagggagattatgacagttggtgaaatgcaacatgcctccgacgaaactaagagactttatacgagtgcttcaagacacgaacttagtgagttatttaacttttcccacactgatgtggggacttcacctttgttccgttacaacttggtcccatttgaactgaaggattggaagattgcccttgctgagctgttcaggtacattaatggtacagattgttggtcaacaatctatctggaaaatcacgaccaacctcgttcaattacgagatttggtgacgattctcccaagaaccgtgttatttctggtaagttactctctgtgttgctaagtgccttgaccggtactctatatgtgtatcagggacaagagcttggccaaatcaatttcaagaactggcctgttgaaaagtacgaggatgtcgaaatcagaaacaactacaatgccattaaagaagagcatggggaaaactcagaggagatgaaaaagtttttagaagccattgcccttatctccagggaccatgctagaacacctatgcaatggtctcgtgaggagccaaatgctggtttttctggtcctagtgctaaaccatggttttacttgaacgactctttcagagaaggcattaacgtcgaagatgaaatcaaggatcccaactcggttttgaacttctggaaggaggccttgaagtttagaaaggcgcataaagacattactgtgtacggatacgatttcgagtttattgatttagacaataagaagttgtttagcttcacaaagaagtacaacaataaaacattgtttgcggctttgaactttagctctgatgcgacagatttcaagattccaaatgatgattcatcgttcaagttagagtttggaaactatccaaagaaggaggtagatgcctcttccagaacattgaagccatgggaaggaagaatatatatcagcgaatga

SEQ ID NO: 43 - DNA, 1770 bp, Saccharomyces cerevisiae
  source 1..1770: mol_type = genomic DNA
atgactatttcttctgcacatccagaaacagaacccaagtggtggaaagaggccacaatctatcaaatttatcccgcaagtttcaaagactccaacaacgatgggtggggtgatatgaagggtattgcctccaagttggagtatatcaaagagcttggtgccgatgccatttggatctcaccattctacgactcgccacaagatgatatgggttacgatattgccaactacgaaaaggtctggccaacctacggtacgaacgaggactgctttgccttgatcgaaaagacacataagcttggtatgaaatttatcaccgacttagtcatcaaccattgctcgagcgaacatgaatggttcaaagagagcagatcctcaaaaaccaatccaaaacgtgactggttcttctggagacctcctaagggttatgacgccgaaggcaagccaattcctccaaacaattggaggtcttacttcggtggttctgcatggacgttcgatgaaaagacacaagagttttacttgcgtttgttttgctccacccaacctgatttgaattgggagaatgaggactgtagaaaggcaatctacgaaagtgccgttggatactggttagaccatggtgtagacggctttagaattgatgtgggaagcttgtactccaaggttgctggtctaccagacgctcctgtgattgacgaaaactcaaagtggcaacccagtgatcctttcacaatgaacggaccacgtatccatgagtttcatcaagaaatgaacaagttcatcagaaacagagtgaaggatggcagagaaattatgacagttggtgaaatgcaacatgctactgatgaaaccaagaggttgtatacaagtgcatcaagacacgaacttagtgagttattcaacttttcccacactgatgtcgggacttcgcccaagttccgtcaaaatttgatcccatatgaactaaaggattggaaggttgcccttgctgaacttttcagatacgttaacggaactgattgttggtcaacaatttatctggaaaatcacgaccaacctcgttcaattaccagatttggtgacgattctcctaagaaccgtgttatttctggtaagttgttgtctgtgttgttggtgtcactgagcggtactctatatgtgtaccaaggacaggaactgggtgaaatcaatttcaagaactggcctatcgaaaaatacgaggatgtcgaagttagaaataactatgatgcgatcaaggaagagcatggagaaaactcgaaggagatgaagaggtttttggaagcaattgcccttatttccagagaccatgctagaacacctatgcaatggtctcgtgaggagccaaatgctggtttttctggtcctaatgctaaaccatggttttacttgaacgagtctttcagggaaggaattaacgctgaagacgaatccaaggatcccaactcggttttgaacttctggaaggaggccttgagatttagaaaggcacacaaggatattactgtatatggatatgattttgagtttattgatttggacaataagaaactgttcagcttcacaaagaaatacgacaacaaaacattgtttgctgctttgaacttcagctctgattcgattgacttcacgattccaaacaatagctcgtcgtttaagttggaattcggaaactacccaagaagcgaagttgatgcgtcttccagaacattgaagccatgggagggcagaatttacatatctgaatga

SEQ ID NO: 44 - DNA, 1770 bp, Saccharomyces cerevisiae
  source 1..1770: mol_type = genomic DNA
atgactatttcttctgcacatccagaaacagaacccaagtggtggaaagaggccacaatttatcaaatttatcccgcaagtttcaaagactccaacaacgatggttggggtgatatgaagggtattgcctccaagttggagtacatcaaagagcttggtaccgatgccatttggatctcgccattttacgactcgccacaagatgatatgggttacgatattgccaactacgaaaaggtctggccaacctacggtacgaacgaggactgctttgccttgatcgaaaagacacataagcttggtatgaaatttatcaccgacttagtcatcaaccattgctccagcgaacatgaatggttcaaagagagcagatcctcaaaaaccaatccaaaacgtgactggttcttctggagacctcctaagggttatgacgccgaaggcaagccaattcctccaaacaattggaggtcttacttcggtggttctgcatggacgttcgatgaaaagacacaagagttttacttgcgtttgttttgctccacccaacctgatctaaactgggagaacgaagactgcagaaaggcaatctacgaaagtgccgttggatactggttagaccatggtgtagacggctttagaattgatgtgggaagcttgtactccaaggttgctggtctaccagacgctcctgtgattgacgaaaactcaaagtggcaactcagtgatcctttcacaatgaacggaccacgtatccatgagtttcatcaagaaatgaacaagttcatcagaaacagagtgaaggatggcagagaaattatgacagttggtgaaatgcgacatgctactgatgagaccaagaggttgtatacaagtgcgtcaagacacgaacttagtgagttattcaacttttcccacactgatgtcgggacttcgcccaagttccgtcaaaatttgataccatatgaactaaaggattggaaggttgcccttgctgaacttttcagatacgttaacggaactgattgttggtcaacaatttatctggaaaatcacgaccaacctcgttcaattaccagatttggtgacgattctcccaaaaaccgtgttatttctggtaagttgttgtctgtgttgttggtgtcactgagcggtactctatatgtgtaccaaggacaggaactgggtgaaatcaatttcaagaactggcctatcgaaaaatacgaggatgtcgaagttagaaataactatgatgcgatcaaggaagagcatggagaaaactcgaaggagatgaagaggtttttggaagcaattgcccttatttccagagaccatgctagaacacctatgcaatggtctcgtgaggagccaaatgctggtttttctggtcctaatgctaaaccatggttttacttgaacgagtctttcagggaaggaattaacgctgaagacgaatccaaggaccccaactcggttttgaacttctggaaggaggccttgagatttagaaaggcacacaaggatattactgtgtatggatatgattttgagtttattgatttggacaataagaaactgttcagcttcacaaagaaatacgacaacaaaacattgtttgctgctttgaacttcagctctgattcgatcgacttcacgattccaaacaatagctcatcgtttaagttggaattcggaaactacccaagaagtgaagttgatgcgtcttccagaacattgaagccatgggagggcagaatttacatatctgaatga

SEQ ID NO: 45 - DNA, 1770 bp, Saccharomyces cerevisiae; identical to SEQ ID NO: 44
  source 1..1770: mol_type = genomic DNA

SEQ ID NO: 46 - DNA, 1746 bp, Saccharomyces cerevisiae
  source 1..1746: mol_type = genomic DNA
atgacgatcatccataatcctaaatggtggaaagaagccaccgtctaccaaatttatcctgctagtttcaaggactccaacaatgacggatggggtgatttagctgggattacctccaaacttgactatgtcaaagaattaggtgtcgatgcgatatgggtgtgcccattttatgactctcctcaagaagatatgggttacgatattgccaactatgagaaggtttggccacgttacggtaccaatgaggattgtttccaaatgatagaagaagctcataagagaggtatcaaggttattgttgacctggttatcaaccactgttctgaagagcatgaatggttcaaggagagtagatcttctaaggctaatccaaagcgtgactggtttttctggagacctccaaagggttacgatgagaaaggtaatccaattcctcccaacaattggagatctttctttggtggatctgcttggagatatgacgaaaaaactggtgagttctttctgcatgtgtttgctcttggccaacctgatttcaattgggaaaatgaagagtgccgtaaagccatttatgactcctcagtgggctattggctacgtcacaacgttgatggctttcgtattgacgtaggcagcatgtattctaaggttgagggcctacccgatgctcctatcactgatcctacagttccttaccaaaaaggaactgaatttttcataaatgggccacgtatccatgagtatcataaagaaatgcataactacatgctctcgcaagttccagagggtaaagaaatcatgactgttggagaagttggtatcgggaatgaagacgacttcagagtttataccagtgccaaagaaggagaactaaatatgatgttcaacttcaagcacacctcagtaggtgaaaacccaaaatgcaagtacgaacttattccctttactttaaaagactttaagctggctcttgctgaaagctttctgtttatcgaaaacacagattgctggtcgaccatctatttggagaatcatgatcaacctcgtagcgtttcgcgttttggctcagattctcctaaatggcgtgagatttcgtccaagatgttggctactctcatcatttcattgactggtactgtttttatctaccaaggacaagaattgggtatgcccaacttcaaaaacagaaaaattgagcaaatcaaatgcgttgaaggtactggtacatatgccgccattaaacgtgattatggtgaagattctgaaaagatgaaaaagttttttgaagcattagctttgatctctagagatcatgggagaacaccattcccatggagtgctgatgagccttctgcaggtttctcgaaggatgccaaaccatggattgacatgaatgaatcatttagagacggaatcaacgcagaagctgagttaaaagataaaaactcagtcttctttttctggaaaaaggccttacaagttagaaaggagcacaaagatatccttgtttatggacataactttcagttcattgatcttgataatgacaagctattcatgtttacaaaggacacagataacaaaaaaatgtttgctgttttcaatttcagtagcgataatacggacttttcagttcctgataatgaggcctcctacaccatgttttttggcaactatgcaaactcaaatggtgattcccgcaccttgcagccatgggaaggaagactttacttgttgaagtaa

SEQ ID NO: 47 - protein, 564 aa, Limosilactobacillus fermentum
MIYTPKWWWQNSVVYQVYPRSFQDSNHDGIGDLKGIISRLDYIKKLGADIIWLNPIYRSPNVDNGYDISDYRAIDPTFGSLTDFKELLTKAHELGLKIMMDLVVNHSSDENEWFKQSRQGKENPYRDYYIWRDPVDGHEPTNWGSYFSGSAWQYDETSGQYYLHLFAVKQPDLNWENEAVRHSVYDIMNWWADLGVDGFRMDVINLISKPAVYKDVPTAPGMQYGDVEPVVANGHRMHEFLQEMHQAVMAKHDLVTVGETPGATTDDAKKYANLEQTELNMVFEFEHVGLDGNDNPALGKWSDKKVSLPELRDNLVKWQTQLNGKAWNSLYWNNHDQPRVVSRFGNDDPKYRVVSAKMLATMLHCLQGTPYIYAGEELGMTNTTFNSLSDYRDLESINAYHQLVDEEHLVDGKTMSRYLAIHSRDNARTPMQWDDSKNAGFSDAEPWIAVNPNYSEINAKAALADPSSVFYHYQKLIQMRHDLPVMTEGKFALVNGNELDEQVFAYTRDDGETTLLVVANFTKETIKREYAAGQGKLLLSNYEDDMGETLRPYEAKVYEFSSKR

SEQ ID NO: 48 - DNA, 1695 bp, Limosilactobacillus fermentum
  source 1..1695: mol_type = genomic DNA
atgatatatacaccaaaatggtggtggcaaaattcagttgtctaccaagtctatccacggagttttcaagacagcaatcatgatggcattggtgatttaaaaggaatcatcagtcggcttgactatattaaaaaactaggtgctgatattatctggctcaatccaatctaccgttcgccaaacgttgataatgggtatgacatcagtgattatcgggcaattgatccaacttttggctcattgaccgactttaaggagctgctgacaaaggctcatgaacttggattaaaaataatgatggatctggtggtcaatcactcatcagatgaaaatgaatggtttaagcaaagccgccaaggaaaagaaaatccataccgtgactattatatctggcgtgatccagtcgatggccatgaaccaactaattggggatcatacttctctggttcagcttggcagtacgatgagacaagcggtcaatactaccttcacctgttcgctgtaaagcagccggatctaaactgggaaaatgaagccgttcgtcattcggtatacgacatcatgaattggtgggcagatctcggtgtcgatggatttcgaatggatgtcatcaacttaatttcaaaaccggctgtctacaaagacgttccaaccgcgcccggaatgcagtatggcgatgtcgagccggttgttgccaatggtcatcggatgcacgaattccttcaagaaatgcatcaagctgtaatggccaagcatgatttggtaacagttggcgaaacacctggtgctacaacagatgatgccaagaagtacgccaaccttgaacagactgagctgaacatggtttttgagtttgagcatgtcggcttagacggtaacgacaacccagctttaggaaaatggagcgacaaaaaagtcagtctgccagaattgcgagataacctggtgaaatggcagactcagctgaatggtaaggcttggaactcgctttactggaacaaccatgatcagccacgcgtcgtatcccgttttggcaatgatgatccaaaatatcgtgtcgtttcggctaaaatgttggcaacaatgctgcactgtctgcaaggcacgccttatatctacgcaggtgaggaactgggaatgaccaacacgacctttaactctttgtctgactatcgtgatttggagagcattaatgcctaccatcagctggttgacgaagagcatctagttgatggaaagacaatgagcagatatctggcaattcattctcgggacaatgccagaacgccaatgcaatgggatgacagcaagaatgccggattttcggatgctgaaccttggatcgcggtcaatcccaactattcagagatcaacgcaaaggcggcactggcggatccatcatccgtcttctatcactaccagaaactcattcaaatgcgccatgacttgccagtaatgacagaaggaaagtttgcgttggtcaatggcaacgaattggatgagcaggtctttgcttacacgcgtgatgatggagaaacaacgctgcttgtagtagccaactttactaaggaaacaatcaagcgagaatacgcggctggtcaaggcaagctcttattaagcaactacgaggatgacatgggagaaactctgcgtccatatgaagctaaagtatatgaattcagttcaaagaggtaa

SEQ ID NO: 49 - protein, 532 aa, synthetic construct
MLLQAFLFLLAGFAAKISASMTNETSDRPLVHFTPNKGWMNDPNGLWYDEKDAKWHLYFQYNPNDTVWGTPLFWGHATSDDLTNWEDQPIAIAPKRNDSGAYSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISAAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 50 - protein, 513 aa, synthetic construct
SMTNETSDRPLVHFTPNKGWMNDPNGLWYDEKDAKWHLYFQYNPNDTVWGTPLFWGHATSDDLTNWEDQPIAIAPKRNDSGAYSGSMVVDYNNTSGFFNDTIDPRQRCVAIWTYNTPESEEQYISYSLDGGYTFTEYQKNPVLAANSTQFRDPKVFWYEPSQKWIMTAAKSQDYKIEIYSSDDLKSWKLESAFANEGFLGYQYECPGLIEVPTEQDPSKSYWVMFISINPGAPAGGSFNQYFVGSFNGTHFEAFDNQSRVVDFGKDYYALQTFFNTDPTYGSALGIAWASNWEYSAFVPTNPWRSSMSLVRKFSLNTEYQANPETELINLKAEPILNISAAGPWSRFATNTTLTKANSYNVDLSNSTGTLEFELVYAVNTTQTISKSVFADLSLWFKGLEDPEEYLRMGFEVSASSFFLDRGNSKVKFVKENPYFTNRMSVNNQPFKSENDLSYYKVYGLLDQNILELYFNDGDVVSTNTYFMTTGNALGSVNMTTGVDNLFYIDKFQVREVK

SEQ ID NO: 51 - protein, 6 aa, synthetic construct
HHHHHH

(19) United States
(12) Patent Application Publication
(10) Pub. No.: US 2026/0009011 A1
(43) Pub. Date: Jan. 8, 2026

(54) METHODS AND COMPOSITIONS FOR TREATING CONGENITAL SUCRASE-ISOMALTASE DEFICIENCY

(71) Applicant: Anagram Therapeutics, Inc., Natick, MA (US)

(72) Inventors: Sridhar Govindarajan, Los Altos, CA (US); Mark Welch, Fremont, CA (US); Alexey Margolin, Newton, MA (US)

(21) Appl. No.: 18/992,837

(22) PCT Filed: Jul. 13, 2023
(86) PCT No.: PCT/US2023/070179

§ 371 Date: Jan. 9, 2025

Related U.S. Application Data
(60) Provisional application No. 63/388,810, filed on Jul. 13, 2022.
(60) Provisional application No. 63/388,845, filed on Jul. 13, 2022.

(51) Int. Cl. C12N 9/26; A61K 9/19; A61K 38/00; C12N 9/24; C12N 15/81

(52) U.S. Cl. CPC C12N 9/2431; A61K 9/19; C12N 9/2402; C12N 15/81; A61K 38/00; C12Y 302/0101; C12Y 302/01026

(57) ABSTRACT

Disclosed are recombinant mutant invertase enzymes. Also disclosed are pharmaceutical compositions comprising an invertase enzyme, e.g., a recombinant mutant invertase enzyme, and an optional isomaltase enzyme. The enzymes and compositions can be used, among other things, to treat congenital sucrase-isomaltase deficiency (CSID).

### CROSS-REFERENCE TO RELATED APPLICATIONS

This application claims the benefit of and priority to U.S. Provisional Patent Application No. 63/388,810, filed on Jul. 13, 2022 and U.S. Provisional Patent Application No. 63/388,845, filed on Jul. 13, 2022, the disclosures of each of which are hereby incorporated by reference in their entireties for all purposes.

### REFERENCE TO A SEQUENCE LISTING XML

This application contains a Sequence Listing which has been submitted electronically in XML format. The Sequence Listing XML is incorporated herein by reference. Said XML file, created on Jul. 12, 2023, is named ANR-004WO_SL.xml and is 80,804 bytes in size.

### FIELD OF THE INVENTION

The invention relates generally to recombinant mutant invertase (sucrase) enzymes and methods and compositions for treating congenital sucrase-isomaltase deficiency (CSID).

### BACKGROUND

Congenital sucrose-isomaltase deficiency (CSID) is a rare autosomal recessive inherited disease of the small intestine resulting from genetic mutations in sucrase-isomaltase, an enzyme complex responsible for catalyzing the hydrolysis of dietary sucrose and starch. Ritz et al. GASTROENTEROLOGY 125 (6): 1678-85. Decreased or absent invertase (sucrase) and/or isomaltase enzymatic activity has been found in patients with CSID, and investigations at the subcellular and molecular levels in intestinal biopsy specimens have led to the description of several phenotypes, differing in transport efficiency, processing, and sorting of the protein, which result in impaired physiologic functions. Ritz et al. (2003) GASTROENTEROLOGY 125 (6): 1678-85, Jacob et al. (2000) J. CLIN. INVEST. 106 (2): 281-7, Alfalah et al. (2009) GASTROENTEROLOGY 136 (3): 883-92, and Ouwendijk (1996) J. CLIN. INVEST. 97 (3): 633-41.

Affected subjects may present with osmotic diarrhea, mild steatorrhea, chronic diarrhea, irritability, and vomiting after consuming sucrose. The difficulty in providing adequate nutrition to these subjects can lead to dehydration, metabolic acidosis, hypercalcemia, failure to thrive, and developmental delay. Cohen (2016) MOL. CELL. PEDIATR. 3:5. Failure to absorb dietary disaccharides and starch has implications for the absorption of other nutrients and the hormonal regulation of gastrointestinal function.

The prevalence of CSID in the European population has been estimated at 1 in 5000, but it is higher among the indigenous populations of Alaska, Greenland, and Canada. Marcadier et al. (2015) CANADIAN MED. ASSOC. J. 187 (2): 102-107. There is also evidence that heterozygous carriers also experience symptoms of CSID, such as chronic diarrhea, abdominal pain, and bloating. It is currently estimated that 2-9% of Americans of European descent may be affected, suggesting that sucrase-isomaltase deficiency has been underrecognized. DeJonge et al. (2014) GASTROENTEROLOGY 146:S705.

CSID currently may be treated by the oral administration of a solution of sacrosidase (SUCRAID®). However, sacrosidase does not fully replace the enzymatic activity of the endogenous sucrase-isomaltase enzyme complex. The liquid formulation of sacrosidase in 50% glycerol is thermally unstable and unstable under acidic conditions. As a result, sacrosidase solution must be discarded four weeks after opening and cannot be used in conjunction with warm beverages and infant formula or fruit juices, negatively impacting ease of use and patient compliance.

Although developments have been made to date, there is still an ongoing need for new and effective pharmaceutical compositions and therapies for treating and managing CSID.

### SUMMARY

The invention is based, in part, upon the development of recombinant mutant invertase (sucrase) enzymes that are active in humans and have greater stability and/or activity than naturally occurring enzymes. In particular, the recombinant mutant enzymes disclosed herein may exhibit improved pH stability, thermal stability, and/or stability against proteolytic digestion compared to naturally occurring versions of the enzyme. Furthermore, the recombinant mutant enzymes disclosed herein may have greater specific activity than a wild type invertase (sucrase) enzyme. Furthermore, it is contemplated that the recombinant mutant enzymes described herein, given their enhanced stability, may be suitable for oral administration, and potentially safer, more tolerable, and/or more active than commercially available invertase (sucrase) enzymes. The invention is also based, in part, upon the development of pharmaceutical compositions, e.g., solid pharmaceutical compositions, comprising an invertase (sucrase) enzyme and an optional isomaltase enzyme that are stable at room temperature, allowing for easier distribution, storage, and administration to subjects relative to compositions that are unstable at room temperature and/or require refrigeration. The recombinant enzymes and compositions can be used, among other things, to treat congenital sucrase-isomaltase deficiency (CSID).

Accordingly, in one aspect, the disclosure relates to a recombinant mutant S. cerevisiae invertase enzyme. In certain embodiments, the invertase has increased activity at acidic pH (e.g., about pH 3.5, 4.0, 4.5, 5.0, 5.5, 6.0, or 6.5) relative to a corresponding wild-type invertase. In certain embodiments, the invertase has increased activity at neutral pH (e.g., about pH 7.0) relative to a corresponding wild-type invertase. In certain embodiments, the invertase has increased stability at acidic pH (e.g., about pH 2.5) relative to a corresponding wild-type invertase. In certain embodiments, the invertase has increased thermal stability relative to a corresponding wild-type invertase. In certain embodiments, the invertase has combination of the above features.

In certain embodiments, the invertase comprises one or more amino acid substitutions selected from the group consisting of: a substitution of an F residue at a position corresponding to position 172 of SEQ ID NO: 2; a substitution of an N residue at a position corresponding to position 429 of SEQ ID NO: 2; a substitution of a P residue at a position corresponding to position 479 of SEQ ID NO: 2; a substitution of a D residue at a position corresponding to position 122 of SEQ ID NO: 2; a substitution of a T residue at a position corresponding to position 140 of SEQ ID NO: 2; a substitution of an E residue at a position corresponding to position 156 of SEQ ID NO: 2; a substitution of a V residue at a position corresponding to position 178 of SEQ ID NO: 2; a substitution of a K residue at a position corresponding to position 259 of SEQ ID NO: 2; a substitution of a D residue at a position corresponding to position 272 of SEQ ID NO: 2; a substitution of a K residue at a position corresponding to position 277 of SEQ ID NO: 2; a substitution of an E residue at a position corresponding to position 285 of SEQ ID NO: 2; a substitution of an A residue at a position corresponding to position 378 of SEQ ID NO: 2; a substitution of an E residue at a position corresponding to position 382 of SEQ ID NO: 2; a substitution of a V residue at a position corresponding to position 399 of SEQ ID NO: 2; a substitution of a G residue at a position corresponding to position 431 of SEQ ID NO: 2; a substitution of a V residue at a position corresponding to position 477 of SEQ ID NO: 2; a substitution of an A residue at a position corresponding to position 501 of SEQ ID NO: 2; a substitution of an L residue at a position corresponding to position 549 of SEQ ID NO: 2; a substitution of a V residue at a position corresponding to position 581 of SEQ ID NO: 2; a substitution of a G residue at a position corresponding to position 586 of SEQ ID NO: 2; or a substitution of an N residue at a position corresponding to position 589 of SEQ ID NO: 2; or a combination of any of the foregoing substitutions. SEQ ID NO: 2 is the amino acid sequence of an exemplary invertase enzyme derived from Saccharomyces cerevisiae, that is wild-type but for the inclusion of a heterologous FAKS signal sequence. Also contemplated herein are invertase enzymes comprising the foregoing substitutions at the corresponding positions in SEQ ID NO: 1 (wild-type invertase enzyme derived from Saccharomyces cerevisiae including a native signal sequence) and SEQ ID NO: 3 (wild-type invertase enzyme derived from Saccharomyces cerevisiae without a signal sequence), see TABLE 1 below.

In certain embodiments, the invertase comprises: a substitution of an F residue at a position corresponding to position 102 of SEQ ID NO: 1; a substitution of a N residue at a position corresponding to position 359 of SEQ ID NO: 1; a substitution of a P residue at a position corresponding to position 409 of SEQ ID NO: 1; a substitution of a D residue at a position corresponding to position 52 of SEQ ID NO: 1; a substitution of a T residue at a position corresponding to position 70 of SEQ ID NO: 1; a substitution of an E residue at a position corresponding to position 86 of SEQ ID NO: 1; a substitution of a V residue at a position corresponding to position 108 of SEQ ID NO: 1; a substitution of a K residue at a position corresponding to position 189 of SEQ ID NO: 1; a substitution of a D residue at a position corresponding to position 202 of SEQ ID NO: 1; a substitution of a K residue at a position corresponding to position 207 of SEQ ID NO: 1; a substitution of an E residue at a position corresponding to position 215 of SEQ ID NO: 1; a substitution of an A residue at a position corresponding to position 308 of SEQ ID NO: 1; a substitution of an E residue at a position corresponding to position 312 of SEQ ID NO: 1; a substitution of a V residue at a position corresponding to position 329 of SEQ ID NO: 1; a substitution of a G residue at a position corresponding to position 361 of SEQ ID NO: 1; a substitution of a V residue at a position corresponding to position 407 of SEQ ID NO: 1; a substitution of an A residue at a position corresponding to position 431 of SEQ ID NO: 1; a substitution of a L residue at a position corresponding to position 479 of SEQ ID NO: 1; a substitution of a V residue at a position corresponding to position 511 of SEQ ID NO: 1; a substitution of a G residue at a position corresponding to position 516 of SEQ ID NO: 1; a substitution of an N residue at a position corresponding to position 519 of SEQ ID NO: 1; or a combination of any of the foregoing substitutions.

In certain embodiments, the invertase comprises: a substitution of an F residue at a position corresponding to position 83 of SEQ ID NO: 3; a substitution of an N residue at a position corresponding to position 340 of SEQ ID NO: 3; a substitution of a P residue at a position corresponding to position 390 of SEQ ID NO: 3; a substitution of a D residue at a position corresponding to position 33 of SEQ ID NO: 3; a substitution of a T residue at a position corresponding to position 51 of SEQ ID NO: 3; a substitution of an E residue at a position corresponding to position 67 of SEQ ID NO: 3; a substitution of a V residue at a position corresponding to position 89 of SEQ ID NO: 3; a substitution of a K residue at a position corresponding to position 170 of SEQ ID NO: 3; a substitution of a D residue at a position corresponding to position 183 of SEQ ID NO: 3; a substitution of a K residue at a position corresponding to position 188 of SEQ ID NO: 3; a substitution of an E residue at a position corresponding to position 196 of SEQ ID NO: 3; a substitution of an A residue at a position corresponding to position 289 of SEQ ID NO: 3; a substitution of an E residue at a position corresponding to position 293 of SEQ ID NO: 3; a substitution of a V residue at a position corresponding to position 310 of SEQ ID NO: 3; a substitution of a G residue at a position corresponding to position 342 of SEQ ID NO: 3; a substitution of a V residue at a position corresponding to position 388 of SEQ ID NO: 3; a substitution of an A residue at a position corresponding to position 412 of SEQ ID NO: 3; a substitution of a L residue at a position corresponding to position 460 of SEQ ID NO: 3; a substitution of a V residue at a position corresponding to position 492 of SEQ ID NO: 3; a substitution of a G residue at a position corresponding to position 497 of SEQ ID NO: 3; a substitution of an N residue at a position corresponding to position 500 of SEQ ID NO: 3; or a combination of any of the foregoing substitutions.

In certain embodiments, in the invertase: the F residue at a position corresponding to position 172 of SEQ ID NO: 2 is substituted by Y (F172Y); the N residue at a position corresponding to position 429 of SEQ ID NO: 2 is substituted by A (N429A); the P residue at a position corresponding to position 479 of SEQ ID NO: 2 is substituted by A (P479A); the D residue at a position corresponding to position 122 of SEQ ID NO: 2 is substituted by E (D122E); the T residue at a position corresponding to position 140 of SEQ ID NO: 2 is substituted by L (T140L); the E residue at a position corresponding to position 156 of SEQ ID NO: 2 is substituted by Q (E156Q); the V residue at a position corresponding to position 178 of SEQ ID NO: 2 is substituted by I (V178I); the K residue at a position corresponding to position 259 of SEQ ID NO: 2 is substituted by L (K259L); the D residue at a position corresponding to position 272 of SEQ ID NO: 2 is substituted by N (D272N); the K residue at a position corresponding to position 277 of SEQ ID NO: 2 is substituted by T (K277T); the E residue at a position corresponding to position 285 of SEQ ID NO: 2 is substituted by H (E285H); the A residue at a position corresponding to position 378 of SEQ ID NO: 2 is substituted by T (A378T); the E residue at a position corresponding to position 382 of SEQ ID NO: 2 is substituted by Q (E382Q); the V residue at a position corresponding to position 399 of SEQ ID NO: 2 is substituted by A (V399A); the G residue at a position corresponding to position 431 of SEQ ID NO: 2 is substituted by R (G431R); the V residue at a position corresponding to position 477 of SEQ ID NO: 2 is substituted by D (V477D); the A residue at a position corresponding to position 501 of SEQ ID NO: 2 is substituted by N (A501N); the L residue at a position corresponding to position 549 of SEQ ID NO: 2 is substituted by I (L549I); the V residue at a position corresponding to position 581 of SEQ ID NO: 2 is substituted by I (V581I); the G residue at a position corresponding to position 586 of SEQ ID NO: 2 is substituted by S (G586S); or the N residue at a position corresponding to position 589 of SEQ ID NO: 2 is substituted by K (N589K); or the invertase comprises a combination of any of the foregoing substitutions. SEQ ID NO: 2 is the amino acid sequence of an exemplary invertase enzyme derived from Saccharomyces cerevisiae, that is wild-type but for the inclusion of a heterologous FAKS signal sequence. Also contemplated herein are invertases comprising the foregoing substitutions at the corresponding positions in SEQ ID NO: 1 (wild-type invertase enzyme derived from Saccharomyces cerevisiae including a native signal sequence) and SEQ ID NO: 3 (wild-type invertase enzyme derived from Saccharomyces cerevisiae without a signal sequence).

In certain embodiments, in the invertase: the F residue at a position corresponding to position 102 of SEQ ID NO: 1 is substituted by Y (F102Y); the N residue at a position corresponding to position 359 of SEQ ID NO: 1 is substituted by A (N359A); the P residue at a position corresponding to position 409 of SEQ ID NO: 1 is substituted by A (P409A); the D residue at a position corresponding to position 52 of SEQ ID NO: 1 is substituted by E (D52E); the A residue at a position corresponding to position 53 of SEQ ID NO: 1 is substituted by G (A53G); the T residue at a position corresponding to position 70 of SEQ ID NO: 1 is substituted by L (T70L); the E residue at a position corresponding to position 86 of SEQ ID NO: 1 is substituted by Q (E86Q); the V residue at a position corresponding to position 108 of SEQ ID NO: 1 is substituted by I (V108I); the K residue at a position corresponding to position 189 of SEQ ID NO: 1 is substituted by L (K189L); the D residue at a position corresponding to position 202 of SEQ ID NO: 1 is substituted by N (D202N); the K residue at a position corresponding to position 207 of SEQ ID NO: 1 is substituted by T (K207T); the E residue at a position corresponding to position 215 of SEQ ID NO: 1 is substituted by H (E215H); the A residue at a position corresponding to position 308 of SEQ ID NO: 1 is substituted by T (A308T); the E residue at a position corresponding to position 312 of SEQ ID NO: 1 is substituted by Q (E312Q); the V residue at a position corresponding to position 329 of SEQ ID NO: 1 is substituted by A (V329A); the G residue at a position corresponding to position 361 of SEQ ID NO: 1 is substituted by R (G361R); the V residue at a position corresponding to position 407 of SEQ ID NO: 1 is substituted by D (V407D); the A residue at a position corresponding to position 431 of SEQ ID NO: 1 is substituted by N (A431N); the L residue at a position corresponding to position 479 of SEQ ID NO: 1 is substituted by I (L479I); the V residue at a position corresponding to position 511 of SEQ ID NO: 1 is substituted by I (V511I); the N residue at a position corresponding to position 519 of SEQ ID NO: 1 is substituted by K (N519K); or the invertase comprises a combination of any of the foregoing substitutions.

In certain embodiments, in the invertase: the F residue at a position corresponding to position 83 of SEQ ID NO: 3 is substituted by Y (F83Y); the N residue at a position corresponding to position 340 of SEQ ID NO: 3 is substituted by A (N340A); the P residue at a position corresponding to position 390 of SEQ ID NO: 3 is substituted by A (P390A); the D residue at a position corresponding to position 33 of SEQ ID NO: 3 is substituted by E (D33E); the T residue at a position corresponding to position 51 of SEQ ID NO: 3 is substituted by L (T51L); the E residue at a position corresponding to position 67 of SEQ ID NO: 3 is substituted by Q (E67Q); the V residue at a position corresponding to position 89 of SEQ ID NO: 3 is substituted by I (V89I); the K residue at a position corresponding to position 170 of SEQ ID NO: 3 is substituted by L (K170L); the D residue at a position corresponding to position 183 of SEQ ID NO: 3 is substituted by N (D183N); the K residue at a position corresponding to position 188 of SEQ ID NO: 3 is substituted by T (K188T); the E residue at a position corresponding to position 196 of SEQ ID NO: 3 is substituted by H (E196H); the A residue at a position corresponding to position 289 of SEQ ID NO: 3 is substituted by T (A289T); the E residue at a position corresponding to position 293 of SEQ ID NO: 3 is substituted by Q (E293Q); the V residue at a position corresponding to position 310 of SEQ ID NO: 3 is substituted by A (V310A); the G residue at a position corresponding to position 342 of SEQ ID NO: 3 is substituted by R (G342R); the V residue at a position corresponding to position 388 of SEQ ID NO: 3 is substituted by D (V388D); the A residue at a position corresponding to position 412 of SEQ ID NO: 3 is substituted by N (A412N); the L residue at a position corresponding to position 460 of SEQ ID NO: 3 is substituted by I (L460I); the V residue at a position corresponding to position 492 of SEQ ID NO: 3 is substituted by I (V492I); the G residue at a position corresponding to position 497 of SEQ ID NO: 3 is substituted by S (G497S); the N residue at a position corresponding to position 500 of SEQ ID NO: 3 is substituted by K (N500K); or the invertase comprises a combination of any of the foregoing substitutions.

In certain embodiments, the invertase comprises one, two, three, four, five, six, seven, eight, nine, ten, or more than ten mutations relative to the corresponding wild-type invertase. In certain embodiments, the invertase comprises the following substitutions at positions corresponding to the amino acids set forth in SEQ ID NO: 2, including: the F172Y, N429A, and P479A substitutions; the E120A, D122E, and P479A substitutions; the K259L, S476N, and K514P substitutions; the T140L, E156Q, and L549I substitutions; the S476A, P479A, and Q597E substitutions; the K277T, E415S, and V581I substitutions; the A123G, T140L, and F143Y substitutions; the N390D, S476N, and K519R substitutions; the E382Q, T573S, and V581I substitutions; the D122E, T470S, and S476N substitutions; the T140Q, P479A, and K514P substitutions; the E120A, V178I, and L549I substitutions; the E382Q, S537D, and N589K substitutions; the T140L, G586S, and F596I substitutions; or the E382Q, L459F, and K519R substitutions.

In certain embodiments, the invertase comprises the following substitutions at positions corresponding to the amino acids set forth in SEQ ID NO: 1, including: the E50A, D52E, and P409A substitutions; the T70L, E86Q, and L479I substitutions; the K189L, S406N, and K444P substitutions; the S406A, P409A, and Q527E substitutions; the K207T, E345S, and V511I substitutions; the A53G, T70L, and F73Y substitutions; the N320D, S406N, and K449R substitutions; the E312Q, T503S, and V511I substitutions; the D52E, T400S, and S406N substitutions; the T70Q, P409A, and K444P substitutions; the F102Y, N359A, and P409A substitutions; the E50A, V108I, and L479I substitutions; the E312Q, S467D, and N519K substitutions; the T70L, G516S, and F526I substitutions; or the E312Q, L389F, and K449R substitutions.

In certain embodiments, the invertase comprises the following substitutions at positions corresponding to the amino acids set forth in SEQ ID NO: 3, including: the E31A, D33E, and P390A substitutions; the T51L, E67Q, and L460I substitutions; the K170L, S387N, and K425P substitutions; the S387A, P390A, and Q508E substitutions; the K188T, E326S, and V492I substitutions; the A34G, T51L, and F54Y substitutions; the N301D, S388N, and K430R substitutions; the E293Q, T484S, and V492I substitutions; the D33E, T381S, and S388N substitutions; the T51L, P390A, and K425P substitutions; the F83Y, N340A, and P390A substitutions; the E31A, V89I, and L460I substitutions; the E293Q, S448D, and N500K substitutions; the T51L, G497S, and F507I substitutions; or the E293Q, L370F, and K430R substitutions.

In certain embodiments, the invertase comprises the amino acid sequence of any one of 29, 19-28, and 30-33, or an amino acid sequence that has at least 85%, 90%, 95%, 96%, 97%, 98%, or 99% sequence identity to any one of SEQ ID NOs: 29, 19-28, and 30-33.

In certain embodiments, the invertase comprises the amino acid sequence of SEQ ID NO: 29, or an amino acid sequence that has at least 85%, 90%, 95%, 96%, 97%, 98%, or 99% sequence identity to SEQ ID NO: 29.

In certain embodiments, the invertase comprises the amino acid sequence of any one of SEQ ID NOs: 14, 4-13, and 15-18, or an amino acid sequence that has at least 85%, 90%, 95%, 96%, 97%, 98%, or 99% sequence identity to any one of SEQ ID NOs: 14, 4-13, and 15-18.

In one aspect, the disclosure relates to a recombinant mutant S. cerevisiae invertase enzyme comprising a substitution, or combination of substitutions, listed in TABLE 1 or TABLE 4.

In certain embodiments, the invertase has a specific activity at about pH 3.5 of at least 800, 900, 1,000, 1,100, 1,200, 1,300, 1,400, 1,500, 1,600, 1,700, 1,800, 1,900, or 2,000 μmol sucrose consumed per minute per milligram of deglycosylated invertase. In certain embodiments, the invertase has at least 1 fold, 1.5 fold, 2 fold, 2.5 fold, or 3 fold higher activity at about pH 3.5, compared to a corresponding wild-type invertase.

In certain embodiments, the invertase has a specific activity at about pH 5.0 of at least 1,000, 1,100, 1,200, 1,300, 1,400, 1,500, 1,600, 1,700, 1,800, 1,900, 2,000, 2,100, or 2,200 μmol sucrose consumed per minute per milligram of deglycosylated invertase. In certain embodiments, the invertase has at least 1 fold, 1.5 fold, 2 fold, 2.5 fold, or 3 fold higher activity at about pH 5.0, compared to a corresponding wild-type invertase.

In certain embodiments, the invertase has a specific activity at about pH 6.0 or 6.2 of at least 700, 800, 900, 1,000, 1,100, 1,200, 1,300, 1,400, 1,500, 1,600, 1,700, or 1,800 μmol sucrose consumed per minute per milligram of deglycosylated invertase. In certain embodiments, the invertase has at least 1 fold, 1.5 fold, 2 fold, 2.5 fold, or 3 fold higher activity at about pH 6.0 or 6.2, compared to a corresponding wild-type invertase.

In certain embodiments, the invertase has a specific activity at about pH 7.0 or 7.1 of at least 200, 300, 400, 500, 600, 700, 800, 900, 1,000, 1,100, 1,200, or 1,300 μmol sucrose consumed per minute per milligram of deglycosylated invertase. In certain embodiments, the invertase has at least 1 fold, 1.5 fold, 2 fold, 2.5 fold, or 3 fold higher activity at about pH 7.0 or 7.1, compared to a corresponding wild-type invertase.

In certain embodiments, the invertase retains at least 20%, 30%, 40%, 50%, 60%, 70%, 80%, 90%, 95%, 96%, 97%, 98%, or 99% of activity following incubation at about pH 2.5 for about 30 minutes. In certain embodiments, the invertase has at least 1 fold, 1.5 fold, 2 fold, 2.5 fold, 3 fold, 3.5 fold, 4 fold, or 5 fold higher stability at about pH 2.5 compared to a corresponding wild-type invertase.

In certain embodiments, the invertase has a Tm of at least 56, 57, 58, 59, 60, 61, 62, 63, 64, 65, 66, 67, or 68° C. In certain embodiments, the invertase has a Tm that is at least 1, 2, 3, 4, 5, 6, 7, 8, 9, or 10° C. higher than a corresponding wild-type invertase.

In certain embodiments, the invertase has higher stability in the presence of pancreatin or pepsin compared to a corresponding wild-type invertase.

In another aspect, the disclosure relates to a nucleic acid encoding an invertase as described herein. In another aspect, the disclosure relates to an expression vector comprising a nucleic acid as described herein. In another aspect, the disclosure relates to a cell comprising an expression vector as described herein. In certain embodiments, the cell is a P. pastoris cell or a S. cerevisiae cell. In another aspect, the disclosure relates to method of producing a recombinant mutant S. cerevisiae invertase enzyme, the method comprising growing a host cell as described herein under conditions so that the host cell expresses the invertase, and purifying the invertase.

In another aspect, the disclosure relates to a pharmaceutical composition comprising an invertase as described herein, and a pharmaceutically acceptable carrier and/or an excipient. Depending upon the circumstances, the invertase can be spray-dried.

In another aspect, the disclosure relates to a pharmaceutical composition comprising: an invertase enzyme, e.g., a recombinant mutant invertase as described herein; a separate isomaltase enzyme; and a pharmaceutically acceptable carrier and/or an excipient. Depending upon the circumstances and the given the pharmaceutical composition of interest, the isomaltase is spray-dried, the invertase is spray-dried, or both the isomaltase and invertase are spray-dried.

In certain embodiments of the pharmaceutical compositions disclosed herein (i) the invertase is a microbial invertase (e.g., derived from Saccharomyces cerevisiae), or a functional fragment or variant thereof, (ii) the invertase comprises a sequence of any one of SEQ ID NOs: 29, 14, 1-13, 15-28, and 30-33, or a functional fragment or variant thereof, (iii) the invertase is a recombinant mutant S. cerevisiae invertase as described herein, (iv) the isomaltase is a microbial isomaltase (e.g., derived from Saccharomyces cerevisiae or Lactobacillus fermentum, or a functional fragment or variant of each of the foregoing), (v) the isomaltase comprises any one of SEQ ID NOs: 37-41, or a functional fragment or variant thereof, (vi) the isomaltase comprises a sequence of SEQ ID NO: 47, (vii) the isomaltase comprises substitutions at positions corresponding to the amino acids sequence set forth in SEQ ID NO: 47, including: the K115I, R132K, D226S, E310A, L366M, I421A, A444G, A531D, and F560V substitutions; the E93K, K115I, R132K, D226S, E310A, L366M, I421A, A444G, A531D, and F560L substitutions; the E93K, K115I, R132K, D226S, E310A, I421A, A444G, E524Q, A531D, and F560L substitutions; the E93K, K115I, R132K, D226S, E310A, I421A, A444G, A531D, and F560V substitutions; the E93K, K115I, R132K, D226S, L366M, I421A, A444G, E524Q, A531D, and F560L substitutions; the E93K, K115I, R132K, D226S, L366M, I421A, A444G, A531D, and F560V substitutions; the E93K, K115I, R132K, D226S, I421A, A444G, E524Q, A531D, and F560V substitutions; the K115I, R132K, D226S, E310A, L366M, I421A, A444G, E524Q, A531D, and F560L substitutions; the K115I, R132K, D226S, E310A, 1421A, A444G, E524Q, A531D, and F560V substitutions; the K115I, R132K, D226S, L366M, I421A, A444G, E524Q, A531D, and F560V substitutions; the E93K, K115I, R132K, A211E, D226S, 1421A, A444G, E524Q, A531D, and F560V substitutions; the E93K, K115I, R132K, D226S, L366M, I421A, A444G, E524Q, A531D, and F560V substitutions; the E93K, K115I, R132K, D226S, E275D, I421A, A444G, E524Q, A531D, and F560V substitutions; the T89A, E93K, K115I, R132K, D226S, I421A, A444G, E524Q, A531D, and F560V substitutions; the E93K, K115I, R132K, H182R, D226S, I421A, A444G, E524Q, A531D, and F560V substitutions; the E93K, K115I, E122D, R132K, D226S, I421A, A444G, E524Q, A531D, and F560V substitutions; the E93K, K115I, D226S, I421A, A444G, E524Q, A531D, and F560V substitutions; the E93K, K115I, D226S, I421A, A444G, E524Q, A531D, and F560L substitutions; the K115I, A211E, D226S, I421A, A444G, A531D, and F560V substitutions; the K115I, D226S, L366M, I421A, A444G, E524Q, A531D, and F560L substitutions; the K115I, A211E, D226S, I421A, A444G, E524Q, A531D, and F560L substitutions; the K115I, R132K, D226S, E310A, L366M, I421A, A444G, A531D, and F560L substitutions; the E93K, K115I, D226S, E310A, I421A, A444G, A531D, and F560L substitutions; the K115I, D226S, E310A, I421A, A444G, A531D, and F560V substitutions; the E93K, K115I, D226S, L366M, I421A, A444G, A531D, and F560L substitutions; the K115I, D226S, I421A, A444G, E524Q, A531D, and F560V substitutions; the K115I, D226S, E310A, I421A, A444G, E524Q, A531D, and F560L substitutions; the K115I, A211E, D226S, L366M, I421A, A444G, A531D, and F560L substitutions; the K115I, A211E, D226S, E310A, 1421A, A444G, A531D, and F560L substitutions; the E93K, K115I, D226S, I421A, A444G, A531D, and F560V substitutions; the E93K, K115I, A211E, D226S, I421A, A444G, A531D, and F560L substitutions; or the K115I, D226S, L366M, I421A, A444G, A531D, and F560V substitutions, (viii) the isomaltase is a recombinant mutant isomaltase set forth in U.S. Provisional Patent Application No. 63/388,845, filed on Jul. 13, 2022, or (ix) or a combination of any one of features (i)-(viii). In certain embodiments, the invertase comprises the amino acid sequence of SEQ ID NO: 14 or 29, or a functional fragment thereof. In certain embodiments, the invertase comprises the amino acid sequence of or SEQ ID NO: 49 or 50, or a functional fragment thereof.

In certain embodiments, the composition is formulated as an oral dosage form. In certain embodiments, the composition is formulated as a powder, satchel, granulate, pellet, micropellet, tablet, or minitablet. In certain embodiments, the composition is formulated as a powder, satchel, or tablet. In certain embodiments, the composition has a shelf-life at room temperature of at least 3 months, 6 months, 9 months, 12 months, 15 months, 18 months, 21 months, 24 months, 36 months, 48 months, 60 months, 72 months, 84 months, 96 months, 108 months, or 120 months.

In another aspect, the disclosure relates to a method of treating congenital sucrase-isomaltase deficiency (CSID) in a subject in need thereof, the method comprising administering, e.g., orally administering, to the subject an enzyme or pharmaceutical composition as described herein. In certain embodiments, the enzyme or composition is administered to the subject together with a meal or snack.

In another aspect, the disclosure relates to a method of reducing sucrose and branched (1-6 linked) α-limit dextrin concentration in a subject, the method comprising administering, e.g., orally administering to the subject an enzyme or pharmaceutical composition as described herein. In certain embodiments, the method reduces sucrose and branched (1-6 linked) α-limit dextrin concentration in the subject as measured by a hydrogen breath test, e.g., a sucrose methane hydrogen breath test or a ¹³C-sucrose breath test.

In another aspect, the disclosure relates to a method of treating congenital sucrase-isomaltase deficiency (CSID) in a subject in need thereof, the method comprising orally administering to the subject (i) an invertase enzyme and (ii) a separate isomaltase enzyme. In certain embodiments, the invertase is a recombinant mutant S. cerevisiae invertase enzyme as described herein. In certain embodiments, the subject is a mammal. In certain embodiments, the subject is a human.

These and other aspects and features of the invention are described in the following detailed description and claims.

### DETAILED DESCRIPTION

The invention is based, in part, upon the discovery of recombinant mutant invertase (sucrase) enzymes that are active in humans and have greater stability and/or activity than naturally occurring enzymes. In particular, the recombinant mutant enzymes of the invention may exhibit improved pH stability, thermal stability, and/or stability against proteolytic digestion compared to naturally occurring versions of the enzyme. Furthermore, the recombinant mutant enzymes of the invention may have greater specific activity than a wild type invertase (sucrase) enzyme. Furthermore, it is contemplated that the recombinant mutant enzymes described herein, given their enhanced stability, may be suitable for oral administration, and potentially safer, more tolerable, and/or more active than commercially available invertase (sucrase) enzymes. The invention is also based, in part, of the discovery of pharmaceutical compositions, e.g., solid pharmaceutical compositions, comprising an invertase (sucrase) enzyme and an isomaltase enzyme that are stable at room temperature, allowing for easier distribution, storage, and administration to subjects relative to compositions that are unstable at room temperature and/or require refrigeration. The recombinant enzymes and compositions can be used, among other things, to treat congenital sucrase-isomaltase deficiency (CSID).

Various features and aspects of the invention are discussed in detail below.

### I. Invertase (Sucrase) Enzymes

Among other things, the invention provides recombinant mutant invertase (sucrase) enzymes and pharmaceutical compositions comprising recombinant mutant invertase (sucrase) enzymes that, for example, are useful in treating disorders such as congenital sucrase-isomaltase deficiency (CSID).

As used herein, the terms “sucrase” and “invertase” are used interchangeably and refer to any enzyme, or a functional fragment thereof, that is capable of catalyzing the hydrolysis of sucrose to fructose and glucose. Sucrases and invertases are also called, acid invertase, alkaline invertase, β-D-fructofuranosidase, β-fructosidase, β-fructofuranosidase, β-fructofuranoside fructohydrolase, β-fructopyranosidase, β-h-fructosidase, β-invertase, EC 3.2.1.26, exo-β-(2,6)-fructofuraosidase, fructosylinvertase, invertin, glucosucrase, saccharase, and sucrose hydrolase, and, unless indicated otherwise, the terms are used interchangeably herein. The term invertase includes variants having one or more amino acid substitutions, deletions, or insertions relative to a wild-type invertase sequence, and/or fusion proteins or conjugates including an invertase.

As used herein, the term “functional fragment” of an invertase refers to fragment of a full-length invertase that retains, for example, at least 10%, at least 20%, at least 30%, at least 40%, at least 50%, at least 60%, at least 70%, at least 80%, at least 90%, or 100% of the enzymatic activity of the corresponding full-length, naturally occurring invertase. Invertase enzymatic activity may be assayed by any method known in the art. Exemplary invertase activity assays are described in Bacon (1955) METHODS IN ENZYMOLOGY 1:258-262, Lever (1972) ANALYTICAL BIOCHEM. 47:273-279, United States Patent Application Publication No. 20140250942, on the world wide web at sigmaaldrich.com/technical-documents/protocols/biology/enzymatic-assay-of-invertase.html, and in Example 1 herein.

Exemplary invertase enzymes include invertase enzymes derived from Saccharomyces cerevisiae. The amino acid sequence of an exemplary wild-type invertase enzyme derived from Saccharomyces cerevisiae (including a native signal sequence) is depicted in SEQ ID NO: 1, and a nucleotide sequence encoding an exemplary wild-type invertase enzyme derived from Saccharomyces cerevisiae is depicted in SEQ ID NO: 35. The amino acid sequence of an exemplary invertase enzyme derived from Saccharomyces cerevisiae (including a heterologous FAKS signal sequence, but otherwise wild-type) is depicted in SEQ ID NO: 2. The amino acid sequence of an exemplary wild-type invertase enzyme derived from Saccharomyces cerevisiae (without a signal sequence) is depicted in SEQ ID NO: 3.

SEQ ID NO: 1 represents wild-type S. cerevisiae invertase with a native signal sequence where the signal sequence is underlined, and the mature protein sequence is italicized:

MLLQAFLFLLAGFAAKISASMTNETSDRPLVHFTPNKGWMNDPNG
LWYDEKDAKWHLYFQYNPNDTVWGTPLFWGHATSDDLTHWEDEPI
AIAPKRNDSGAFSGSMVVDYNNTSGFENDTIDPRQRCVAIWTYNT
PESEEQYISYSLDGGYTFTEYQKNPVLAANSTQFRDPKVFWYEPS
QKWIMTAAKSQDYKIEIYSSDDLKSWKLESAFANEGFLGYQYECP
GLIEVPTEQDPSKSYWVMFISINPGAPAGGSFNQYFVGSFNGTHF
EAFDNQSRVVDFGKDYYALQTFFNTDPTYGSALGIAWASNWEYSA
FVPTNPWRSSMSLVRKESLNTEYQANPETELINLKAEPILNISNA
GPWSRFATNTTLTKANSYNVDLSNSTGTLEFELVYAVNTTQTISK
SVFPDLSLWFKGLEDPEEYLRMGFEASASSFFLDRGNSKVKFVKE
NPYFTNRMSVNNQPFKSENDLSYYKVYGLLDQNILELYENDGDVV
STNTYFMTTGNALGSVNMTTGVDNLFYIDKFQVREVK.

SEQ ID NO: 2 represents wild-type S. cerevisiae invertase with a heterologous FAKS signal sequence, where the signal sequence is underlined, and the mature protein sequence is italicized:

MRFPSIFTAVLFAASSALAAPVNTTTEDETAQIPAEAVIGYSDLE
GDEDVAVLPFSNSTNNGLLFINTTIASIAAKEEGVSLEKREAEAS
MTNETSDRPLVHFTPNKGWMNDPNGLWYDEKDAKWHLYFQYNPND
TVWGTPLFWGHATSDDLTHWEDEPIAIAPKRNDSGAFSGSMVVDY
NNTSGFFNDTIDPRQRCVAIWTYNTPESEEQYISYSLDGGYTFTE
YQKNPVLAANSTQFRDPKVEWYEPSQKWIMTAAKSQDYKIEIYSS
DDLKSWKLESAFANEGELGYQYECPGLIEVPTEQDPSKSYWVMFI
SINPGAPAGGSFNQYFVGSENGTHFEAFDNQSRVVDFGKDYYALQ
TFFNTDPTYGSALGIAWASNWEYSAFVPTNPWRSSMSLVRKFSLN
TEYQANPETELINLKAEPILNISNAGPWSRFATNTTLTKANSYNV
DLSNSTGTLEFELVYAVNTTQTISKSVFPDLSLWFKGLEDPEEYL
RMGFEASASSFFLDRGNSKVKFVKENPYFTNRMSVNNQPFKSEND
LSYYKVYGLLDQNILELYFNDGDVVSTNTYFMTTGNALGSVNMTT
GVDNLFYIDKFQVREVK.

SEQ ID NO: 3 represents wild-type S. cerevisiae invertase without a signal sequence:

SMTNETSDRPLVHFTPNKGWMNDPNGLWYDEKDAKWHLYFQYNPN
DTVWGTPLFWGHATSDDLTHWEDEPIAIAPKRNDSGAFSGSMVVD
YNNTSGFENDTIDPRQRCVAIWTYNTPESEEQYISYSLDGGYTFT
EYQKNPVLAANSTQFRDPKVFWYEPSQKWIMTAAKSQDYKIEIYS
SDDLKSWKLESAFANEGFLGYQYECPGLIEVPTEQDPSKSYWVMF
ISINPGAPAGGSFNQYFVGSENGTHFEAFDNQSRVVDFGKDYYAL
QTFFNTDPTYGSALGIAWASNWEYSAFVPTNPWRSSMSLVRKFSL
NTEYQANPETELINLKAEPILNISNAGPWSRFATNTTLTKANSYN
VDLSNSTGTLEFELVYAVNTTQTISKSVFPDLSLWFKGLEDPEEY
LRMGFEASASSFFLDRGNSKVKFVKENPYFTNRMSVNNQPFKSEN
DLSYYKVYGLLDQNILELYFNDGDVVSTNTYFMTTGNALGSVNMT
TGVDNLFYIDKFQVREVK.

A contemplated invertase enzyme may comprise the amino acid sequence of SEQ ID NO: 1, SEQ ID NO: 2, or SEQ ID NO: 3 or an amino acid sequence that comprises at least one (e.g., one, two, three, four, five, six, seven, eight, nine or ten, 2-10, 3-10, 4-10, 5-10, 6-10, 7-10, 8-10, 9-10, 2-9, 3-9, 4-9, 5-9, 6-9, 7-9, 8-9, 2-8, 3-8, 4-8, 5-8, 6-8, 7-8, 2-6, 3-6, 4-6, or 5-6) mutation(s) and has at least 80%, 85%, 90%, 95%, 96%, 97%, 98%, or 99% sequence identity to SEQ ID NO: 1, SEQ ID NO: 2, or SEQ ID NO: 3.

Additional exemplary invertase enzymes can be found on the world wide web at brenda-enzymes.org/enzyme.php?ecno=3.2.1.26&onlyTable=Sequence. Additional exemplary invertase enzymes are described in United States Patent Application Publication No. 2014/0250942.

Among other things, the invention provides recombinant mutant invertases that are useful, for example, in treating disorders associated with a reduced ability to digest or absorb dietary sucrose and starches.

In certain embodiments, the invertase comprises: (i) increased activity at acidic pH (e.g., about pH 3.5, 4.0, 4.5, 5.0, 5.5, 6.0, or 6.5) relative to a corresponding wild-type invertase; (ii) increased activity at neutral pH (e.g., about pH 7.0) relative to a corresponding wild-type invertase; (iii) increased stability at acidic pH (e.g., about pH 2.5) relative to a corresponding wild-type invertase, (iv) increased thermal stability relative to a corresponding wild-type invertase, or any combination of features (i), (ii), (iii), or (iv). Exemplary combinations include (i) and (ii); (i) and (iii); (i) and (iv); (ii) and (iii); (ii) and (iv); (i), (ii) and (iii); (i), (ii) and (iv); (i), (iii) and (iv); (ii), (iii) and (iv); and (i), (ii), (iii) and (iv).

In certain embodiments, the recombinant mutant invertase comprises at least one (e.g., one, two, three, four, five, six, seven, eight, nine or ten, 2-10, 3-10, 4-10, 5-10, 6-10, 7-10, 8-10, 9-10, 2-9, 3-9, 4-9, 5-9, 6-9, 7-9, 8-9, 2-8, 3-8, 4-8, 5-8, 6-8, 7-8, 2-6, 3-6, 4-6, or 5-6) mutation(s) at a position corresponding to wild type S. cerevisiae invertase of SEQ ID NO: 2. In certain embodiments, at least one mutation is selected from a substitution of a residue at a position corresponding to position to position 122 of SEQ ID NO: 2; a substitution of a T residue at a position corresponding to position 140 of SEQ ID NO: 2; a substitution of an E residue at a position corresponding to position 156 of SEQ ID NO: 2; a substitution of an F residue at a position corresponding to position 172 of SEQ ID NO: 2; a substitution of a V residue at a position corresponding to position 178 of SEQ ID NO: 2; a substitution of a K residue at a position corresponding to position 259 of SEQ ID NO: 2; a substitution of a D residue at a position corresponding to position 272 of SEQ ID NO: 2; a substitution of a K residue at a position corresponding to position 277 of SEQ ID NO: 2; a substitution of an E residue at a position corresponding to position 285 of SEQ ID NO: 2; a substitution of an A residue at a position corresponding to position 378 of SEQ ID NO: 2; a substitution of an E residue at a position corresponding to position 382 of SEQ ID NO: 2; a substitution of a V residue at a position corresponding to position 399 of SEQ ID NO: 2; a substitution of a G residue at a position corresponding to position 431 of SEQ ID NO: 2; a substitution of a V residue at a position corresponding to position 477 of SEQ ID NO: 2; a substitution of a P residue at a position corresponding to position 479 of SEQ ID NO: 2; a substitution of an A residue at a position corresponding to position 501 of SEQ ID NO: 2; a substitution of an L residue at a position corresponding to position 549 of SEQ ID NO: 2; a substitution of a V residue at a position corresponding to position 581 of SEQ ID NO: 2; a substitution of a G residue at a position corresponding to position 586 of SEQ ID NO: 2; or a substitution of an N residue at a position corresponding to position 589 of SEQ ID NO: 2. In certain embodiments, the recombinant mutant invertase comprises a combination of any of the foregoing substitutions. SEQ ID NO: 2 is the amino acid sequence of an exemplary invertase enzyme derived from Saccharomyces cerevisiae, that is wild-type but for the inclusion of a heterologous FAKS signal sequence. Also contemplated herein are invertases comprising the foregoing substitutions at the corresponding positions in SEQ ID NO: 1 (wild-type invertase enzyme derived from Saccharomyces cerevisiae including a native signal sequence) and in SEQ ID NO: 3 (wild-type invertase enzyme derived from Saccharomyces cerevisiae without a signal sequence).

In certain embodiments, the invertase comprises: a substitution of a D residue at a position corresponding to position 52 of SEQ ID NO: 1; a substitution of a T residue at a position corresponding to position 70 of SEQ ID NO: 1; a substitution of an E residue at a position corresponding to position 86 of SEQ ID NO: 1; a substitution of an F residue at a position corresponding to position 102 of SEQ ID NO: 1; a substitution of a V residue at a position corresponding to position 108 of SEQ ID NO: 1; a substitution of a K residue at a position corresponding to position 189 of SEQ ID NO: 1; a substitution of a D residue at a position corresponding to position 202 of SEQ ID NO: 1; a substitution of a K residue at a position corresponding to position 207 of SEQ ID NO: 1; a substitution of an E residue at a position corresponding to position 215 of SEQ ID NO: 1; a substitution of an A residue at a position corresponding to position 308 of SEQ ID NO: 1; a substitution of an E residue at a position corresponding to position 312 of SEQ ID NO: 1; a substitution of a V residue at a position corresponding to position 329 of SEQ ID NO: 1; a substitution of a G residue at a position corresponding to position 361 of SEQ ID NO: 1; a substitution of a V residue at a position corresponding to position 407 of SEQ ID NO: 1; a substitution of a P residue at a position corresponding to position 409 of SEQ ID NO: 1; a substitution of an A residue at a position corresponding to position 431 of SEQ ID NO: 1; a substitution of a L residue at a position corresponding to position 479 of SEQ ID NO: 1; a substitution of a V residue at a position corresponding to position 511 of SEQ ID NO: 1; a substitution of a G residue at a position corresponding to position 516 of SEQ ID NO: 1; or a substitution of an N residue at a position corresponding to position 519 of SEQ ID NO: 1. In certain embodiments, the recombinant mutant invertase comprises a combination of any of the foregoing substitutions.

In certain embodiments, the invertase comprises: a substitution of a D residue at a position corresponding to position 33 of SEQ ID NO: 3; a substitution of a T residue at a position corresponding to position 51 of SEQ ID NO: 3; a substitution of an E residue at a position corresponding to position 67 of SEQ ID NO: 3; a substitution of an F residue at a position corresponding to position 83 of SEQ ID NO: 3; a substitution of a V residue at a position corresponding to position 89 of SEQ ID NO: 3; a substitution of a K residue at a position corresponding to position 170 of SEQ ID NO: 3; a substitution of a D residue at a position corresponding to position 183 of SEQ ID NO: 3; a substitution of a K residue at a position corresponding to position 188 of SEQ ID NO: 3; a substitution of an E residue at a position corresponding to position 196 of SEQ ID NO: 3; a substitution of an A residue at a position corresponding to position 289 of SEQ ID NO: 3; a substitution of an E residue at a position corresponding to position 293 of SEQ ID NO: 3; a substitution of a V residue at a position corresponding to position 310 of SEQ ID NO: 3; a substitution of a G residue at a position corresponding to position 342 of SEQ ID NO: 3; a substitution of a V residue at a position corresponding to position 388 of SEQ ID NO: 3; a substitution of a P residue at a position corresponding to position 390 of SEQ ID NO: 3; a substitution of an A residue at a position corresponding to position 412 of SEQ ID NO: 3; a substitution of a L residue at a position corresponding to position 460 of SEQ ID NO: 3; a substitution of a V residue at a position corresponding to position 492 of SEQ ID NO: 3; a substitution of a G residue at a position corresponding to position 497 of SEQ ID NO: 3; or a substitution of an N residue at a position corresponding to position 500 of SEQ ID NO: 3. In certain embodiments, the recombinant mutant invertase comprises a combination of any of the foregoing substitutions.

In certain embodiments, the D residue at a position corresponding to position 122 of SEQ ID NO: 2 is substituted by E (D122E); the T residue at a position corresponding to position 140 of SEQ ID NO: 2 is substituted by L (T140L); the E residue at a position corresponding to position 156 of SEQ ID NO: 2 is substituted by Q (E156Q); the F residue at a position corresponding to position 172 of SEQ ID NO: 2 is substituted by Y (F172Y); the V residue at a position corresponding to position 178 of SEQ ID NO: 2 is substituted by I (V178I); the K residue at a position corresponding to position 259 of SEQ ID NO: 2 is substituted by L (K259L); the D residue at a position corresponding to position 272 of SEQ ID NO: 2 is substituted by N (D272N); the K residue at a position corresponding to position 277 of SEQ ID NO: 2 is substituted by T (K277T); the E residue at a position corresponding to position 285 of SEQ ID NO: 2 is substituted by H (E285H); the A residue at a position corresponding to position 378 of SEQ ID NO: 2 is substituted by T (A378T); the E residue at a position corresponding to position 382 of SEQ ID NO: 2 is substituted by Q (E382Q); the V residue at a position corresponding to position 399 of SEQ ID NO: 2 is substituted by A (V399A); the G residue at a position corresponding to position 431 of SEQ ID NO: 2 is substituted by R (G431R); the V residue at a position corresponding to position 477 of SEQ ID NO: 2 is substituted by D (V477D); the P residue at a position corresponding to position 479 of SEQ ID NO: 2 is substituted by A (P479A); the A residue at a position corresponding to position 501 of SEQ ID NO: 2 is substituted by N (A501N); the L residue at a position corresponding to position 549 of SEQ ID NO: 2 is substituted by I (L549I); the V residue at a position corresponding to position 581 of SEQ ID NO: 2 is substituted by I (V581I); the G residue at a position corresponding to position 586 of SEQ ID NO: 2 is substituted by S (G586S); or the N residue at a position corresponding to position 589 of SEQ ID NO: 2 is substituted by K (N589K); or the invertase comprises a combination of any of the foregoing substitutions. SEQ ID NO: 2 is the amino acid sequence of an exemplary invertase enzyme derived from Saccharomyces cerevisiae, that is wild-type but for the inclusion of a heterologous FAKS signal sequence. Also contemplated herein are invertases comprising the foregoing substitutions at the corresponding positions in SEQ ID NO: 1 (wild-type invertase enzyme derived from Saccharomyces cerevisiae including a native signal sequence) and SEQ ID NO: 3 (wild-type invertase enzyme derived from Saccharomyces cerevisiae without a signal sequence).

In certain embodiments, in the invertase: the D residue at a position corresponding to position 52 of SEQ ID NO: 1 is substituted by E (D52E); the A residue at a position corresponding to position 53 of SEQ ID NO: 1 is substituted by G (A53G); the T residue at a position corresponding to position 70 of SEQ ID NO: 1 is substituted by L (T70L); the E residue at a position corresponding to position 86 of SEQ ID NO: 1 is substituted by Q (E86Q); the F residue at a position corresponding to position 102 of SEQ ID NO: 1 is substituted by Y (F102Y); the V residue at a position corresponding to position 108 of SEQ ID NO: 1 is substituted by I (V108I); the K residue at a position corresponding to position 189 of SEQ ID NO: 1 is substituted by L (K189L); the D residue at a position corresponding to position 202 of SEQ ID NO: 1 is substituted by N (D202N); the K residue at a position corresponding to position 207 of SEQ ID NO: 1 is substituted by T (K207T); the E residue at a position corresponding to position 215 of SEQ ID NO: 1 is substituted by H (E215H); the A residue at a position corresponding to position 308 of SEQ ID NO: 1 is substituted by T (A308T); the E residue at a position corresponding to position 312 of SEQ ID NO: 1 is substituted by Q (E312Q); the V residue at a position corresponding to position 329 of SEQ ID NO: 1 is substituted by A (V329A); the G residue at a position corresponding to position 361 of SEQ ID NO: 1 is substituted by R (G361R); the V residue at a position corresponding to position 407 of SEQ ID NO: 1 is substituted by D (V407D); the P residue at a position corresponding to position 409 of SEQ ID NO: 1 is substituted by A (P409A); the A residue at a position corresponding to position 431 of SEQ ID NO: 1 is substituted by N (A431N); the L residue at a position corresponding to position 479 of SEQ ID NO: 1 is substituted by I (L479I); the V residue at a position corresponding to position 511 of SEQ ID NO: 1 is substituted by I (V511I); the N residue at a position corresponding to position 519 of SEQ ID NO: 1 is substituted by K (N519K); or the invertase comprises a combination of any of the foregoing substitutions.

In certain embodiments, in the invertase: the D residue at a position corresponding to position 33 of SEQ ID NO: 3 is substituted by E (D33E); the T residue at a position corresponding to position 51 of SEQ ID NO: 3 is substituted by L (T51L); the E residue at a position corresponding to position 67 of SEQ ID NO: 3 is substituted by Q (E67Q); the F residue at a position corresponding to position 83 of SEQ ID NO: 3 is substituted by Y (F83Y); the V residue at a position corresponding to position 89 of SEQ ID NO: 3 is substituted by I (V89I); the K residue at a position corresponding to position 170 of SEQ ID NO: 3 is substituted by L (K170L); the D residue at a position corresponding to position 183 of SEQ ID NO: 3 is substituted by N (D183N); the K residue at a position corresponding to position 188 of SEQ ID NO: 3 is substituted by T (K188T); the E residue at a position corresponding to position 196 of SEQ ID NO: 3 is substituted by H (E196H); the A residue at a position corresponding to position 289 of SEQ ID NO: 3 is substituted by T (A289T); the E residue at a position corresponding to position 293 of SEQ ID NO: 3 is substituted by Q (E293Q); the V residue at a position corresponding to position 310 of SEQ ID NO: 3 is substituted by A (V310A); the G residue at a position corresponding to position 342 of SEQ ID NO: 3 is substituted by R (G342R); the V residue at a position corresponding to position 388 of SEQ ID NO: 3 is substituted by D (V388D); the P residue at a position corresponding to position 390 of SEQ ID NO: 3 is substituted by A (P390A); the A residue at a position corresponding to position 412 of SEQ ID NO: 3 is substituted by N (A412N); the L residue at a position corresponding to position 460 of SEQ ID NO: 3 is substituted by I (L460I); the V residue at a position corresponding to position 492 of SEQ ID NO: 3 is substituted by I (V492I); the G residue at a position corresponding to position 497 of SEQ ID NO: 3 is substituted by S (G497S); the N residue at a position corresponding to position 500 of SEQ ID NO: 3 is substituted by K (N500K); or the invertase comprises a combination of any of the foregoing substitutions.

In certain embodiments, one or more mutations may be conservative substitutions relative to wild type S. cerevisiae invertase of SEQ ID NO: 1, SEQ ID NO: 2, or SEQ ID NO: 3, whereas in certain other embodiments, one or more mutations may be non-conservative substitutions relative to wild type S. cerevisiae invertase of SEQ ID NO: 1, SEQ ID NO: 2, or SEQ ID NO: 3. As used herein, the term “conservative substitution” refers to a substitution with a structurally and/or functionally similar amino acid.

In certain embodiments, the substitution of a given amino acid is with a hydrophobic amino acid (e.g., A, I, L, M, or V), a positively charged amino acid (e.g., K, R or H), a negatively charged amino acid (e.g., D or E), a polar neutral amino acid (e.g., N, C, Q, S or T), an aromatic amino acid (e.g., F, Y or W) or a bulkier amino acid based on side chain volume or a smaller amino acid based on side chain volume. Conservative substitutions may also be defined by the BLAST (Basic Local Alignment Search Tool) algorithm, the BLOSUM substitution matrix (e.g., BLOSUM 62 matrix), or the PAM substitution:p matrix (e.g., the PAM 250 matrix). Non-conservative substitutions are amino acid substitutions that are not conservative substitutions.

In certain embodiments, the recombinant mutant invertase enzyme comprises one or more substitutions from TABLE 1, wherein the positions of the substitutions are shown relative to SEQ ID NO: 1 (wild-type Saccharomyces cerevisiae invertase enzyme, including a native signal sequence) or SEQ ID NO: 2 (Saccharomyces cerevisiae invertase enzyme including a heterologous FAKS signal sequence, but otherwise wild-type) or SEQ ID NO: 3 (wild-type Saccharomyces cerevisiae invertase enzyme without a signal sequence).

TABLE 1

| Position relative | Position relative | Position relative | Exemplary |
|---|---|---|---|
| to SEQ ID NO: 2 | to SEQ ID NO: 1 | to SEQ ID NO: 3 | Substitutions |
| E120 | E50 | E31 | A |
| D122 | D52 | D33 | E |
| A123 | A53 | A34 | G |
| T140 | T70 | T51 | L, Q |
| F143 | F73 | F54 | Y |
| E156 | E86 | E67 | Q |
| F172 | F102 | F83 | Y |
| V178 | V108 | V89 | I |
| K259 | K189 | K170 | L |
| K277 | K207 | K188 | T |
| L359 | L289 | L270 | F |
| T365 | T295 | T276 | S |
| A378 | A308 | A289 | T |
| E382 | E312 | E293 | Q |
| N390 | N320 | N301 | D |
| E415 | E345 | E326 | S |
| L416 | L346 | L327 | Q |
| N429 | N359 | N340 | A |
| L459 | L389 | L370 | F |
| T470 | T400 | T381 | S |
| S476 | S406 | S387 | A, N |
| P479 | P409 | P390 | A |
| N512 | N442 | N423 | G |
| K514 | K444 | K425 | P |
| K519 | K449 | K430 | R |
| S537 | S467 | S448 | D |
| L549 | L479 | L460 | I |
| T573 | T503 | T484 | S |
| V581 | V511 | V492 | I |
| G586 | G516 | G497 | S |
| N589 | N519 | N500 | K |
| F596 | F526 | F507 | I |
| Q597 | Q527 | Q508 | E |

In certain embodiments, the recombinant mutant invertase comprises at least one (e.g., at least one, at least two, at least three, at least four, at least five, at least six, at least seven, at least eight, at least nine, at least ten, or at least eleven) mutation(s) selected from 5 TABLE 1.

In certain embodiments, the recombinant mutant invertase comprises at least three substitutions selected from: the F172Y, N429A, and P479A substitutions relative to SEQ ID NO: 2; the E120A, D122E, and P479A substitutions relative to SEQ ID NO: 2; the K259L, S476N, and K514P substitutions relative to SEQ ID NO: 2; the T140L, E156Q, and L549I substitutions relative to SEQ ID NO: 2; the S476A, P479A, and Q597E substitutions relative to SEQ ID NO: 2; the K277T, E415S, and V581I substitutions relative to SEQ ID NO: 2; the A123G, T140L, and F143Y substitutions relative to SEQ ID NO: 2; the N390D, S476N, and K519R substitutions relative to SEQ ID NO: 2; the E382Q, T573S, and V581I substitutions relative to SEQ ID NO: 2; the D122E, T470S, and S476N substitutions relative to SEQ ID NO: 2; the T140Q, P479A, and K514P substitutions relative to SEQ ID NO: 2; the E120A, V178I, and L549I substitutions relative to SEQ ID NO: 2; the E382Q, S537D, and N589K substitutions relative to SEQ ID NO: 2; the T140L, G586S, and F596I substitutions relative to SEQ ID NO: 2; or the E382Q, L459F, and K519R substitutions relative to SEQ ID NO: 2. It is understood, the recombinant mutant invertase can comprise the same groups of three substitutions noted above with reference to SEQ ID NO:5, as well as invertases containing the same groups of three substitutions but with reference to SEQ ID NO: 1 or SEQ ID NO: 3.

In certain embodiments, the invertase comprises the following substitutions at positions corresponding to the amino acids set forth in SEQ ID NO: 1, including: the E50A, D52E, and P409A substitutions relative to SEQ ID NO: 1; the T70L, E86Q, and L479I substitutions relative to SEQ ID NO: 1; the K189L, S406N, and K444P substitutions relative to SEQ ID NO: 1; the S406A, P409A, and Q527E substitutions relative to SEQ ID NO: 1; the K207T, E345S, and V511I substitutions relative to SEQ ID NO: 1; the A53G, T70L, and F73Y substitutions relative to SEQ ID NO: 1; the N320D, S406N, and K449R substitutions relative to SEQ ID NO: 1; the E312Q, T503S, and V511I substitutions relative to SEQ ID NO: 1; the D52E, T400S, and S406N substitutions relative to SEQ ID NO: 1; the T70Q, P409A, and K444P substitutions relative to SEQ ID NO: 1; the F102Y, N359A, and P409A substitutions relative to SEQ ID NO: 1; the E50A, V108I, and L479I substitutions relative to SEQ ID NO: 1; the E312Q, S467D, and N519K substitutions relative to SEQ ID NO: 1; the T70L, G516S, and F526I substitutions relative to SEQ ID NO: 1; or the E312Q, L389F, and K449R substitutions relative to SEQ ID NO: 1.

In certain embodiments, the recombinant mutant invertase comprises at least three substitutions selected from: the E31A, D33E, and P390A substitutions relative to SEQ ID NO: 3; the T51L, E67Q, and L460I substitutions relative to SEQ ID NO: 3; the K170L, S387N, and K425P substitutions relative to SEQ ID NO: 3; the S387A, P390A, and Q508E substitutions relative to SEQ ID NO: 3; the K188T, E326S, and V492I substitutions relative to SEQ ID NO: 3; the A34G, T51L, and F54Y substitutions relative to SEQ ID NO: 3; the N301D, S388N, and K430R substitutions relative to SEQ ID NO: 3; the E293Q, T484S, and V492I substitutions relative to SEQ ID NO: 3; the D33E, T381S, and S388N substitutions relative to SEQ ID NO: 3; the T51L, P390A, and K425P substitutions relative to SEQ ID NO: 3; the F83Y, N340A, and P390A substitutions relative to SEQ ID NO: 3; the E31A, V89I, and L460I substitutions relative to SEQ ID NO: 3; the E293Q, S448D, and N500K substitutions relative to SEQ ID NO: 3; the T51L, G497S, and F507I substitutions relative to SEQ ID NO: 3; or the E293Q, L370F, and K430R substitutions relative to SEQ ID NO: 3.

In certain embodiments, the recombinant mutant invertase comprises the E120A, D122E, and P479A substitutions (relative to SEQ ID NO: 2). For example, the recombinant mutant invertase may comprise SEQ ID NO: 4, also referred to as V6 herein.

SEQ ID NO: 4
MRFPSIFTAVLFAASSALAAPVNTTTEDETAQIPAEAVIGYSDLE
GDEDVAVLPFSNSINNGLLFINTTIASIAAKEEGVSLEKREAEAS
MTNETSDRPLVHFTPNKGWMNDPNGLWYDAKEAKWHLYFQYNPND
TVWGTPLFWGHATSDDLTHWEDEPIAIAPKRNDSGAFSGSMVVDY
NNTSGFFNDTIDPRQRCVAIWTYNTPESEEQYISYSLDGGYTFTE
YQKNPVLAANSTQFRDPKVEWYEPSQKWIMTAAKSQDYKIEIYSS
DDLKSWKLESAFANEGFLGYQYECPGLIEVPTEQDPSKSYWVMFI
SINPGAPAGGSFNQYFVGSENGTHFEAFDNQSRVVDFGKDYYALQ
TFFNTDPTYGSALGIAWASNWEYSAFVPTNPWRSSMSLVRKESLN
TEYQANPETELINLKAEPILNISNAGPWSRFATNTTLTKANSYNV
DLSNSTGTLEFELVYAVNTTQTISKSVFADLSLWEKGLEDPEEYL
RMGFEASASSFFLDRGNSKVKFVKENPYFTNRMSVNNQPFKSEND
LSYYKVYGLLDQNILELYFNDGDVVSTNTYFMTTGNALGSVNMTT
GVDNLFYIDKFQVREVK.

In certain embodiments, the recombinant mutant invertase comprises the T140L, E156Q, and L549I substitutions relative to SEQ ID NO: 2. For example, the recombinant mutant invertase may comprise SEQ ID NO: 5, also referred to as V9 herein.

SEQ ID NO: 5
MRFPSIFTAVLFAASSALAAPVNTTTEDETAQIPAEAVIGYSDLE
GDEDVAVLPFSNSINNGLLFINTTIASIAAKEEGVSLEKREAEAS
MTNETSDRPLVHFTPNKGWMNDPNGLWYDEKDAKWHLYFQYNPND
TVWGLPLFWGHATSDDLTHWQDEPIAIAPKRNDSGAFSGSMVVDY
NNTSGFFNDTIDPRQRCVAIWTYNTPESEEQYISYSLDGGYTFTE
YQKNPVLAANSTQFRDPKVEWYEPSQKWIMTAAKSQDYKIEIYSS
DDLKSWKLESAFANEGFLGYQYECPGLIEVPTEQDPSKSYWVMFI
SINPGAPAGGSFNQYFVGSENGTHFEAFDNQSRVVDFGKDYYALQ
TFENTDPTYGSALGIAWASNWEYSAFVPTNPWRSSMSLVRKESLN
TEYQANPETELINLKAEPILNISNAGPWSRFATNTTLTKANSYNV
DLSNSTGTLEFELVYAVNTTQTISKSVEPDLSLWFKGLEDPEEYL
RMGFEASASSFFLDRGNSKVKFVKENPYFTNRMSVNNQPFKSEND
LSYYKVYGILDQNILELYFNDGDVVSTNTYFMTTGNALGSVNMTT
GVDNLFYIDKFQVREVK.

In certain embodiments, the recombinant mutant invertase comprises the S476A, P479A, and Q597E substitutions relative to SEQ ID NO: 2. For example, the recombinant mutant invertase may comprise SEQ ID NO: 6, also referred to as V14 herein.

SEQ ID NO: 6
MRFPSIFTAVLFAASSALAAPVNTTTEDETAQIPAEAVIGYSDLE
GDEDVAVLPESNSTNNGLLFINTTIASIAAKEEGVSLEKREAEAS
MTNETSDRPLVHFTPNKGWMNDPNGLWYDEKDAKWHLYFQYNPND
TVWGTPLFWGHATSDDLTHWEDEPIAIAPKRNDSGAFSGSMVVDY
NNTSGFFNDTIDPRQRCVAIWTYNTPESEEQYISYSLDGGYTFTE
YQKNPVLAANSTQFRDPKVFWYEPSQKWIMTAAKSQDYKIEIYSS
DDLKSWKLESAFANEGFLGYQYECPGLIEVPTEQDPSKSYWVMFI
SINPGAPAGGSFNQYFVGSENGTHFEAFDNQSRVVDFGKDYYALQ
TFFNTDPTYGSALGIAWASNWEYSAFVPTNPWRSSMSLVRKFSLN
TEYQANPETELINLKAEPILNISNAGPWSRFATNTTLTKANSYNV
DLSNSTGTLEFELVYAVNTTQTISKAVFADLSLWFKGLEDPEEYL
RMGFEASASSFFLDRGNSKVKFVKENPYFTNRMSVNNQPFKSEND
LSYYKVYGLLDQNILELYFNDGDVVSTNTYFMTTGNALGSVNMTT
GVDNLFYIDKFEVREVK.

In certain embodiments, the recombinant mutant invertase comprises the K259L, S476N, and K514P substitutions (relative to SEQ ID NO: 2). For example, the recombinant mutant invertase may comprise SEQ ID NO: 7, also referred to as V22 herein.

SEQ ID NO: 7
MRFPSIFTAVLFAASSALAAPVNTTTEDETAQIPAEAVIGYSDLE
GDEDVAVLPFSNSTNNGLLFINTTIASIAAKEEGVSLEKREAEAS
MTNETSDRPLVHFTPNKGWMNDPNGLWYDEKDAKWHLYFQYNPND
TVWGTPLFWGHATSDDLTHWEDEPIAIAPKRNDSGAFSGSMVVDY
NNTSGFFNDTIDPRQRCVAIWTYNTPESEEQYISYSLDGGYTFTE
YQKNPVLAANSTQFRDPKVFWYEPSQKWIMTAALSQDYKIEIYSS
DDLKSWKLESAFANEGFLGYQYECPGLIEVPTEQDPSKSYWVMFI
SINPGAPAGGSFNQYFVGSENGTHFEAFDNQSRVVDEGKDYYALQ
TFFNTDPTYGSALGIAWASNWEYSAFVPTNPWRSSMSLVRKESLN
TEYQANPETELINLKAEPILNISNAGPWSRFATNTTLTKANSYNV
DLSNSTGTLEFELVYAVNTTQTISKNVFPDLSLWFKGLEDPEEYL
RMGFEASASSFFLDRGNSPVKFVKENPYFTNRMSVNNQPFKSEND
LSYYKVYGLLDQNILELYFNDGDVVSTNTYFMTTGNALGSVNMTT
GVDNLFYIDKFQVREVK.

In certain embodiments, the recombinant mutant invertase comprises the K277T, E415S, and V581I substitutions relative to SEQ ID NO: 2. For example, the recombinant mutant invertase may comprise SEQ ID NO: 8, also referred to as V25 herein.

SEQ ID NO: 8
MRFPSIFTAVLFAASSALAAPVNTTTEDETAQIPAEAVIGYSDLEGDEDVAVLPESNSTNNG
LLFINTTIASIAAKEEGVSLEKREAEASMTNETSDRPLVHFTPNKGWMNDPNGLWYDEKDAK
WHLYFQYNPNDTVWGTPLFWGHATSDDLTHWEDEPIAIAPKRNDSGAFSGSMVVDYNNTSGF
FNDTIDPRQRCVAIWTYNTPESEEQYISYSLDGGYTFTEYQKNPVLAANSTQFRDPKVEWYE
PSQKWIMTAAKSQDYKIEIYSSDDLKSWTLESAFANEGFLGYQYECPGLIEVPTEQDPSKSY
WVMFISINPGAPAGGSFNQYFVGSENGTHFEAFDNOSRVVDFGKDYYALQTFENTDPTYGSA
LGIAWASNWEYSAFVPTNPWRSSMSLVRKESLNTEYQANPETSLINLKAEPILNISNAGPWS
RFATNTTLTKANSYNVDLSNSTGTLEFELVYAVNTTQTISKSVFPDLSLWFKGLEDPEEYLR
MGFEASASSFFLDRGNSKVKFVKENPYFTNRMSVNNQPFKSENDLSYYKVYGLLDONILELY
FNDGDVVSTNTYFMTTGNALGSINMTTGVDNLFYIDKFQVREVK.

In certain embodiments, the recombinant mutant invertase comprises the A123G, T140L, and F143Y substitutions relative to SEQ ID NO: 2. For example, the recombinant mutant invertase may comprise SEQ ID NO: 9, also referred to as V32 herein.

SEQ ID NO: 9
MRFPSIFTAVLFAASSALAAPVNTTTEDETAQIPAEAVIGYSDLEGDEDVAVLPFSNSINNG
LLFINTTIASIAAKEEGVSLEKREAEASMTNETSDRPLVHFTPNKGWMNDPNGLWYDEKDGK
WHLYFQYNPNDTVWGLPLYWGHATSDDLTHWEDEPIAIAPKRNDSGAFSGSMVVDYNNTSGF
FNDTIDPRORCVAIWTYNTPESEEQYISYSLDGGYTFTEYQKNPVLAANSTOFRDPKVEWYE
PSQKWIMTAAKSQDYKIEIYSSDDLKSWKLESAFANEGFLGYQYECPGLIEVPTEQDPSKSY
WVMFISINPGAPAGGSFNQYFVGSFNGTHFEAFDNQSRVVDFGKDYYALQTFFNTDPTYGSA
LGIAWASNWEYSAFVPTNPWRSSMSLVRKESLNTEYQANPETELINLKAEPILNISNAGPWS
RFATNTTLTKANSYNVDLSNSTGTLEFELVYAVNTTQTISKSVFPDLSLWFKGLEDPEEYLR
MGFEASASSFFLDRGNSKVKFVKENPYFTNRMSVNNOPFKSENDLSYYKVYGLLDONILELY
FNDGDVVSTNTYFMTTGNALGSVNMTTGVDNLFYIDKFQVREVK.

In certain embodiments, the recombinant mutant invertase comprises the N390D, S476N, and K519R substitutions relative to SEQ ID NO: 2. For example, the recombinant mutant invertase may comprise SEQ ID NO: 10, also referred to as V38 herein.

SEQ ID NO: 10
MRFPSIFTAVLFAASSALAAPVNTTTEDETAQIPAEAVIGYSDLEGDEDVAVLPFSNSTNNG
LLFINTTIASIAAKEEGVSLEKREAEASMTNETSDRPLVHFTPNKGWMNDPNGLWYDEKDAK
WHLYFQYNPNDTVWGTPLFWGHATSDDLTHWEDEPIAIAPKRNDSGAFSGSMVVDYNNTSGF
FNDTIDPRORCVAIWTYNTPESEEQYISYSLDGGYTFTEYQKNPVLAANSTOFRDPKVEWYE
PSQKWIMTAAKSQDYKIEIYSSDDLKSWKLESAFANEGFLGYQYECPGLIEVPTEQDPSKSY
WVMFISINPGAPAGGSFNQYFVGSFNGTHFEAFDNQSRVVDFGKDYYALQTFFNTDPTYGSA
LGIAWASNWEYSAFVPTDPWRSSMSLVRKESLNTEYQANPETELINLKAEPILNISNAGPWS
RFATNTTLTKANSYNVDLSNSTGTLEFELVYAVNTTQTISKNVEPDLSLWFKGLEDPEEYLR
MGFEASASSFFLDRGNSKVKFVRENPYFTNRMSVNNQPFKSENDLSYYKVYGLLDONILELY
FNDGDVVSTNTYFMTTGNALGSVNMTTGVDNLFYIDKFQVREVK.

In certain embodiments, the recombinant mutant invertase comprises the E382Q, T573S, and V581I substitutions relative to SEQ ID NO: 2. For example, the recombinant mutant invertase may comprise SEQ ID NO: 11, also referred to as V60 herein.

SEQ ID NO: 11
MRFPSIFTAVLFAASSALAAPVNTTTEDETAQIPAEAVIGYSDLEGDFDVAVLPESNSINNG
LLFINTTIASIAAKEEGVSLEKREAEASMTNETSDRPLVHFTPNKGWMNDPNGLWYDEKDAK
WHLYFQYNPNDTVWGTPLFWGHATSDDLTHWEDEPIAIAPKRNDSGAFSGSMVVDYNNTSGF
FNDTIDPRORCVAIWTYNTPESEEQYISYSLDGGYTFTEYQKNPVLAANSTQFRDPKVEWYE
PSQKWIMTAAKSQDYKIEIYSSDDLKSWKLESAFANEGFLGYQYECPGLIEVPTEQDPSKSY
WVMFISINPGAPAGGSFNQYFVGSFNGTHFEAFDNQSRVVDFGKDYYALQTFFNTDPTYGSA
LGIAWASNWQYSAFVPTNPWRSSMSLVRKESLNTEYOANPETELINLKAEPILNISNAGPWS
RFATNTTLTKANSYNVDLSNSTGTLEFELVYAVNTTQTISKSVEPDLSLWEKGLEDPEEYLR
MGFEASASSFFLDRGNSKVKFVKENPYFTNRMSVNNQPFKSENDLSYYKVYGLLDONILELY
FNDGDVVSTNTYFMSTGNALGSINMTTGVDNLFYIDKFOVREVK.

In certain embodiments, the recombinant mutant invertase comprises the D122E, T470S, and S476N substitutions relative to SEQ ID NO: 2. For example, the recombinant mutant invertase may comprise SEQ ID NO: 12, also referred to as V63 herein.

SEQ ID NO: 12
MRFPSIFTAVLFAASSALAAPVNTTTEDETAQIPAEAVIGYSDLEGDEDVAVLPFSNSINNG
LLFINTTIASIAAKEEGVSLEKREAEASMTNETSDRPLVHFTPNKGWMNDPNGLWYDEKEAK
WHLYFQYNPNDTVWGTPLFWGHATSDDLTHWEDEPIAIAPKRNDSGAFSGSMVVDYNNTSGF
FNDTIDPRORCVAIWTYNTPESEEQYISYSLDGGYTFTEYQKNPVLAANSTOFRDPKVEWYE
PSQKWIMTAAKSQDYKIEIYSSDDLKSWKLESAFANEGELGYQYECPGLIEVPTEQDPSKSY
WVMFISINPGAPAGGSFNQYFVGSENGTHFEAFDNOSRVVDEGKDYYALQTFENTDPTYGSA
LGIAWASNWEYSAFVPTNPWRSSMSLVRKESLNTEYQANPETELINLKAEPILNISNAGPWS
RFATNTTLTKANSYNVDLSNSTGTLEFELVYAVNTSQTISKNVFPDLSLWFKGLEDPEEYLR
MGFEASASSFFLDRGNSKVKFVKENPYFTNRMSVNNQPFKSENDLSYYKVYGLLDONILELY
FNDGDVVSTNTYFMTTGNALGSVNMTTGVDNLFYIDKFQVREVK.

In certain embodiments, the recombinant mutant invertase comprises the T140Q, P479A, and K514P substitutions relative to SEQ ID NO: 2. For example, the recombinant mutant invertase may comprise SEQ ID NO: 13, also referred to as V70 herein.

SEQ ID NO: 13
MRFPSIFTAVLFAASSALAAPVNTTTEDETAQIPAEAVIGYSDLEGDEDVAVLPFSNSINNG
LLFINTTIASIAAKEEGVSLEKREAEASMTNETSDRPLVHFTPNKGWMNDPNGLWYDEKDAK
WHLYFQYNPNDTVWGQPLFWGHATSDDLTHWEDEPIAIAPKRNDSGAFSGSMVVDYNNTSGF
FNDTIDPRORCVAIWTYNTPESEEQYISYSLDGGYTFTEYQKNPVLAANSTOFRDPKVEWYE
PSQKWIMTAAKSQDYKIEIYSSDDLKSWKLESAFANEGELGYQYECPGLIEVPTEQDPSKSY
WVMFISINPGAPAGGSFNQYFVGSENGTHFEAFDNQSRVVDEGKDYYALQTFENTDPTYGSA
LGIAWASNWEYSAFVPTNPWRSSMSLVRKESLNTEYQANPETELINLKAEPILNISNAGPWS
RFATNTTLTKANSYNVDLSNSTGTLEFELVYAVNTTQTISKSVFADLSLWFKGLEDPEEYLR
MGFEASASSFFLDRGNSPVKFVKENPYFTNRMSVNNQPFKSENDLSYYKVYGLLDONILELY
FNDGDVVSTNTYFMTTGNALGSVNMTTGVDNLFYIDKFQVREVK.

In certain embodiments, the recombinant mutant invertase comprises the F172Y, N429A, and P479A substitutions (relative to SEQ ID NO: 2). For example, the recombinant mutant invertase may comprise SEQ ID NO: 14, also referred to as V78 herein.

SEQ ID NO: 14
MRFPSIFTAVLFAASSALAAPVNTTTEDETAQIPAEAVIGYSDLEGDEDVAVLPFSNSINNG
LLFINTTIASIAAKEEGVSLEKREAEASMTNETSDRPLVHFTPNKGWMNDPNGLWYDEKDAK
WHLYFQYNPNDTVWGTPLFWGHATSDDLTHWEDEPIAIAPKRNDSGAYSGSMVVDYNNTSGF
FNDTIDPRORCVAIWTYNTPESEEQYISYSLDGGYTFTEYOKNPVLAANSTOFRDPKVFWYE
PSQKWIMTAAKSQDYKIEIYSSDDLKSWKLESAFANEGFLGYQYECPGLIEVPTEQDPSKSY
WVMFISINPGAPAGGSFNQYFVGSFNGTHFEAFDNQSRVVDFGKDYYALQTFFNTDPTYGSA
LGIAWASNWEYSAFVPTNPWRSSMSLVRKESLNTEYQANPETELINLKAEPILNISAAGPWS
RFATNTTLTKANSYNVDLSNSTGTLEFELVYAVNTTQTISKSVFADLSLWEKGLEDPEEYLR
MGFEASASSFFLDRGNSKVKFVKENPYFTNRMSVNNQPFKSENDLSYYKVYGLLDONILELY
FNDGDVVSTNTYFMTTGNALGSVNMTTGVDNLFYIDKFQVREVK.

In certain embodiments, the recombinant mutant invertase comprises the E120A, V178I, and L549I substitutions relative to SEQ ID NO: 2. For example, the recombinant mutant invertase may comprise SEQ ID NO: 15, also referred to as V83 herein.

SEQ ID NO: 15
MRFPSIFTAVLFAASSALAAPVNTTTEDETAQIPAEAVIGYSDLEGDEDVAVLPFSNSTNNG
LLFINTTIASIAAKEEGVSLEKREAEASMTNETSDRPLVHFTPNKGWMNDPNGLWYDAKDAK
WHLYFQYNPNDTVWGTPLFWGHATSDDLTHWEDEPIAIAPKRNDSGAFSGSMVIDYNNTSGF
FNDTIDPRORCVAIWTYNTPESEEQYISYSLDGGYTFTEYQKNPVLAANSTOFRDPKVEWYE
PSQKWIMTAAKSQDYKIEIYSSDDLKSWKLESAFANEGFLGYQYECPGLIEVPTEQDPSKSY
WVMFISINPGAPAGGSFNQYFVGSFNGTHFEAFDNOSRVVDFGKDYYALQTFENTDPTYGSA
LGIAWASNWEYSAFVPTNPWRSSMSLVRKFSLNTEYQANPETELINLKAEPILNISNAGPWS
RFATNTTLTKANSYNVDLSNSTGTLEFELVYAVNTTOTISKSVFPDLSLWFKGLEDPEEYLR
MGFEASASSFFLDRGNSKVKFVKENPYFTNRMSVNNQPFKSENDLSYYKVYGILDQNILELY
FNDGDVVSTNTYFMTTGNALGSVNMTTGVDNLFYIDKFQVREVK.

In certain embodiments, the recombinant mutant invertase comprises the E382Q, S537D, and N589K substitutions relative to SEQ ID NO: 2. For example, the recombinant mutant invertase may comprise SEQ ID NO: 16, also referred to as V87 herein.

SEQ ID NO: 16
MRFPSIFTAVLFAASSALAAPVNTTTEDETAQIPAEAVIGYSDLEGDFDVAVLPFSNSINNG
LLFINTTIASIAAKEEGVSLEKREAEASMTNETSDRPLVHFTPNKGWMNDPNGLWYDEKDAK
WHLYFQYNPNDTVWGTPLFWGHATSDDLTHWEDEPIAIAPKRNDSGAFSGSMVVDYNNTSGF
FNDTIDPRORCVAIWTYNTPESEEQYISYSLDGGYTFTEYQKNPVLAANSTQFRDPKVEWYE
PSQKWIMTAAKSQDYKIEIYSSDDLKSWKLESAFANEGFLGYQYECPGLIEVPTEQDPSKSY
WVMFISINPGAPAGGSFNQYFVGSFNGTHFEAFDNQSRVVDFGKDYYALQTFENTDPTYGSA
LGIAWASNWQYSAFVPTNPWRSSMSLVRKESLNTEYQANPETELINLKAEPILNISNAGPWS
RFATNTTLTKANSYNVDLSNSTGTLEFELVYAVNTTQTISKSVEPDLSLWFKGLEDPEEYLR
MGFEASASSFFLDRGNSKVKFVKENPYFTNRMSVNNQPFKDENDLSYYKVYGLLDONILELY
FNDGDVVSTNTYFMTTGNALGSVNMTTGVDKLFYIDKFQVREVK.

In certain embodiments, the recombinant mutant invertase comprises the T140L, G586S, and F596I substitutions relative to SEQ ID NO: 2. For example, the recombinant mutant invertase may comprise SEQ ID NO: 17, also referred to as V89 herein.

SEQ ID NO: 17
MRFPSIFTAVLFAASSALAAPVNTTTEDETAQIPAEAVIGYSDLEGDEDVAVLPFSNSINNG
LLFINTTIASIAAKEEGVSLEKREAEASMTNETSDRPLVHFTPNKGWMNDPNGLWYDEKDAK
WHLYFQYNPNDTVWGLPLFWGHATSDDLTHWEDEPIAIAPKRNDSGAFSGSMVVDYNNTSGF
FNDTIDPRORCVAIWTYNTPESEEQYISYSLDGGYTFTEYQKNPVLAANSTOFRDPKVFWYE
PSQKWIMTAAKSQDYKIEIYSSDDLKSWKLESAFANEGFLGYQYECPGLIEVPTEQDPSKSY
WVMFISINPGAPAGGSFNQYFVGSFNGTHFEAFDNOSRVVDFGKDYYALQTFFNTDPTYGSA
LGIAWASNWEYSAFVPTNPWRSSMSLVRKESLNTEYQANPETELINLKAEPILNISNAGPWS
RFATNTTLTKANSYNVDLSNSTGTLEFELVYAVNTTQTISKSVFPDLSLWFKGLEDPEEYLR
MGFEASASSFFLDRGNSKVKFVKENPYFTNRMSVNNOPFKSENDLSYYKVYGLLDONILELY
FNDGDVVSTNTYFMTTGNALGSVNMTTSVDNLFYIDKIQVREVK.

In certain embodiments, the recombinant mutant invertase comprises the E382Q, L459F, and K519R substitutions relative to SEQ ID NO: 2. For example, the recombinant mutant invertase may comprise SEQ ID NO: 18, also referred to as V90 herein.

SEQ ID NO: 18
MRFPSIFTAVLFAASSALAAPVNTTTEDETAQIPAEAVIGYSDLEGDEDVAVLPFSNSINNG
LLFINTTIASIAAKEEGVSLEKREAEASMTNETSDRPLVHFTPNKGWMNDPNGLWYDEKDAK
WHLYFQYNPNDTVWGTPLFWGHATSDDLTHWEDEPIAIAPKRNDSGAFSGSMVVDYNNTSGF
FNDTIDPRORCVAIWTYNTPESEEQYISYSLDGGYTFTEYQKNPVLAANSTOFRDPKVEWYE
PSQKWIMTAAKSQDYKIEIYSSDDLKSWKLESAFANEGFLGYQYECPGLIEVPTEQDPSKSY
WVMFISINPGAPAGGSFNQYFVGSENGTHFEAFDNQSRVVDFGKDYYALQTFENTDPTYGSA
LGIAWASNWQYSAFVPTNPWRSSMSLVRKESLNTEYQANPETELINLKAEPILNISNAGPWS
RFATNTTLTKANSYNVDLSNSTGTFEFELVYAVNTTQTISKSVFPDLSLWFKGLEDPEEYLR
MGFEASASSFFLDRGNSKVKFVRENPYFTNRMSVNNQPFKSENDLSYYKVYGLLDONILELY
FNDGDVVSTNTYFMTTGNALGSVNMTTGVDNLFYIDKFQVREVK.

In certain embodiments, the recombinant mutant invertase comprises the E31A, D33E, and P390A substitutions (relative to SEQ ID NO: 3). For example, the recombinant mutant invertase may comprise SEQ ID NO: 19, also referred to as V6 herein.

SEQ ID NO: 19
SMTNETSDRPLVHFTPNKGWMNDPNGLWYDAKEAKWHLYFQYNPNDTVWGTPLFWGHATSDD
LTHWEDEPIAIAPKRNDSGAFSGSMVVDYNNTSGFFNDTIDPRQRCVAIWTYNTPESEEQYI
SYSLDGGYTFTEYQKNPVLAANSTQFRDPKVFWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNOSRVVDFGKDYYALQTFFNTDPTYGSALGIAWASNWEYSAFVPTNPWRSSMSLV
RKFSLNTEYQANPETELINLKAEPILNISNAGPWSRFATNTTLTKANSYNVDLSNSTGTLEF
ELVYAVNTTQTISKSVFADLSLWFKGLEDPEEYLRMGFEASASSFFLDRGNSKVKFVKENPY
FTNRMSVNNQPFKSENDLSYYKVYGLLDQNILELYENDGDVVSTNTYFMTTGNALGSVNMTT
GVDNLFYIDKFQVREVK.

In certain embodiments, the recombinant mutant invertase comprises the T51L, E67Q, and L460I substitutions relative to SEQ ID NO: 3. For example, the recombinant mutant invertase may comprise SEQ ID NO: 20, also referred to as V9 herein.

SEQ ID NO: 20
SMTNETSDRPLVHFTPNKGWMNDPNGLWYDEKDAKWHLYFQYNPNDTVWGLPLEWGHATSDD
LTHWQDEPIAIAPKRNDSGAFSGSMVVDYNNTSGFENDTIDPRORCVAIWTYNTPESEEQYI
SYSLDGGYTFTEYQKNPVLAANSTOFRDPKVFWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDFGKDYYALQTFFNTDPTYGSALGIAWASNWEYSAFVPTNPWRSSMSLV
RKFSLNTEYQANPETELINLKAEPILNISNAGPWSRFATNTTLTKANSYNVDLSNSTGTLEF
ELVYAVNTTQTISKSVFPDLSLWFKGLEDPEEYLRMGFEASASSFFLDRGNSKVKFVKENPY
FTNRMSVNNQPFKSENDLSYYKVYGILDQNILELYFNDGDVVSTNTYFMTTGNALGSVNMTT
GVDNLFYIDKFQVREVK.

In certain embodiments, the recombinant mutant invertase comprises the S387A, P390A, and Q508E substitutions relative to SEQ ID NO: 3. For example, the recombinant mutant invertase may comprise SEQ ID NO: 21, also referred to as V14 herein.

SEQ ID NO: 21
SMTNETSDRPLVHFTPNKGWMNDPNGLWYDEKDAKWHLYFQYNPNDTVWGTPLFWGHATSDD
LTHWEDEPIAIAPKRNDSGAFSGSMVVDYNNTSGFENDTIDPRQRCVAIWTYNTPESEEQYI
SYSLDGGYTFTEYQKNPVLAANSTQFRDPKVEWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDFGKDYYALQTFFNTDPTYGSALGIAWASNWEYSAFVPTNPWRSSMSLV
RKFSLNTEYQANPETELINLKAEPILNISNAGPWSRFATNTTLTKANSYNVDLSNSTGTLEF
ELVYAVNTTQTISKAVFADLSLWEKGLEDPEEYLRMGFEASASSFFLDRGNSKVKFVKENPY
FTNRMSVNNQPFKSENDLSYYKVYGLLDQNILELYFNDGDVVSTNTYFMTTGNALGSVNMTT
GVDNLFYIDKFEVREVK.

In certain embodiments, the recombinant mutant invertase comprises the K170L, S387N, and K425P substitutions (relative to SEQ ID NO: 3). For example, the recombinant mutant invertase may comprise SEQ ID NO: 22, also referred to as V22 herein.

SEQ ID NO: 22
SMTNETSDRPLVHFTPNKGWMNDPNGLWYDEKDAKWHLYFQYNPNDTVWGTPLFWGHATSDD
LTHWEDEPIAIAPKRNDSGAFSGSMVVDYNNTSGFENDTIDPRQRCVAIWTYNTPESEEQYI
SYSLDGGYTFTEYQKNPVLAANSTQFRDPKVEWYEPSQKWIMTAALSQDYKIEIYSSDDLKS
WKLESAFANEGFLGYQYECPGLIEVPTEQDPSKSYWVMFISINPGAPAGGSFNQYFVGSENG
THFEAFDNQSRVVDEGKDYYALQTFFNTDPTYGSALGIAWASNWEYSAFVPTNPWRSSMSLV
RKFSLNTEYQANPETELINLKAEPILNISNAGPWSRFATNTTLTKANSYNVDLSNSTGTLEF
ELVYAVNTTQTISKNVFPDLSLWFKGLEDPEEYLRMGFEASASSFFLDRGNSPVKFVKENPY
FTNRMSVNNQPFKSENDLSYYKVYGLLDONILELYENDGDVVSTNTYFMTTGNALGSVNMTT
GVDNLFYIDKFQVREVK.

In certain embodiments, the recombinant mutant invertase comprises the K188T, E326S, and V492I substitutions relative to SEQ ID NO: 3. For example, the recombinant mutant invertase may comprise SEQ ID NO: 23, also referred to as V25 herein.

SEQ ID NO: 23
SMTNETSDRPLVHFTPNKGWMNDPNGLWYDEKDAKWHLYFQYNPNDTVWG
TPLFWGHATSDDLTHWEDEPIAIAPKRNDSGAFSGSMVVDYNNTSGFEND
TIDPRQRCVAIWTYNTPESEEQYISYSLDGGYTFTEYQKNPVLAANSTQF
RDPKVFWYEPSQKWIMTAAKSQDYKIEIYSSDDLKSWTLESAFANEGFLG
YQYECPGLIEVPTEQDPSKSYWVMFISINPGAPAGGSFNQYFVGSENGTH
FEAFDNQSRVVDFGKDYYALQTFFNTDPTYGSALGIAWASNWEYSAFVPT
NPWRSSMSLVRKFSLNTEYQANPETSLINLKAEPILNISNAGPWSRFATN
TTLTKANSYNVDLSNSTGTLEFELVYAVNTTQTISKSVFPDLSLWFKGLE
DPEEYLRMGFEASASSFFLDRGNSKVKFVKENPYFTNRMSVNNQPFKSEN
DLSYYKVYGLLDQNILELYFNDGDVVSTNTYFMTTGNALGSINMTTGVDN
LFYIDKFQVREVK.

In certain embodiments, the recombinant mutant invertase comprises the A34G, T51L, and F54Y substitutions relative to SEQ ID NO: 3. For example, the recombinant mutant invertase may comprise SEQ ID NO: 24, also referred to as V32 herein.

SEQ ID NO: 24
SMTNETSDRPLVHFTPNKGWMNDPNGLWYDEKDGKWHLYFQYNPNDTVWGLPLYWGHATSDD
LTHWEDEPIAIAPKRNDSGAFSGSMVVDYNNTSGFENDTIDPRQRCVAIWTYNTPESEEQYI
SYSLDGGYTFTEYQKNPVLAANSTQFRDPKVEWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDFGKDYYALQTFFNTDPTYGSALGIAWASNWEYSAFVPTNPWRSSMSLV
RKFSLNTEYQANPETELINLKAEPILNISNAGPWSRFATNTTLTKANSYNVDLSNSTGTLEF
ELVYAVNTTQTISKSVFPDLSLWFKGLEDPEEYLRMGFEASASSFFLDRGNSKVKFVKENPY
FTNRMSVNNQPFKSENDLSYYKVYGLLDQNILELYFNDGDVVSTNTYFMTTGNALGSVNMTT
GVDNLFYIDKFQVREVK.

In certain embodiments, the recombinant mutant invertase comprises the N301D, S388N, and K430R substitutions relative to SEQ ID NO: 3. For example, the recombinant mutant invertase may comprise SEQ ID NO: 25, also referred to as V38 herein.

SEQ ID NO: 25
SMTNETSDRPLVHFTPNKGWMNDPNGLWYDEKDAKWHLYFQYNPNDTVWGTPLFWGHATSDD
LTHWEDEPIAIAPKRNDSGAFSGSMVVDYNNTSGFENDTIDPRQRCVAIWTYNTPESEEQYI
SYSLDGGYTFTEYQKNPVLAANSTQFRDPKVEWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDFGKDYYALQTFFNTDPTYGSALGIAWASNWEYSAFVPTDPWRSSMSLV
RKFSLNTEYQANPETELINLKAEPILNISNAGPWSRFATNTTLTKANSYNVDLSNSTGTLEF
ELVYAVNTTQTISKNVFPDLSLWEKGLEDPEEYLRMGFEASASSFFLDRGNSKVKEVRENPY
FTNRMSVNNQPFKSENDLSYYKVYGLLDQNILELYENDGDVVSTNTYFMTTGNALGSVNMTT
GVDNLFYIDKFQVREVK.

In certain embodiments, the recombinant mutant invertase comprises the E293Q, T484S, and V492I substitutions relative to SEQ ID NO: 3. For example, the recombinant mutant invertase may comprise SEQ ID NO: 26, also referred to as V60 herein.

SEQ ID NO: 26
SMTNETSDRPLVHFTPNKGWMNDPNGLWYDEKDAKWHLYFQYNPNDTVWGTPLFWGHATSDD
LTHWEDEPIAIAPKRNDSGAFSGSMVVDYNNTSGFENDTIDPRQRCVAIWTYNTPESEEQYI
SYSLDGGYTFTEYQKNPVLAANSTQFRDPKVEWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDFGKDYYALQTFFNTDPTYGSALGIAWASNWQYSAFVPTNPWRSSMSLV
RKFSLNTEYQANPETELINLKAEPILNISNAGPWSRFATNTTLTKANSYNVDLSNSTGTLEF
ELVYAVNTTQTISKSVFPDLSLWFKGLEDPEEYLRMGFEASASSFFLDRGNSKVKFVKENPY
FTNRMSVNNQPFKSENDLSYYKVYGLLDQNILELYFNDGDVVSTNTYEMSTGNALGSINMTT
GVDNLFYIDKFQVREVK.

In certain embodiments, the recombinant mutant invertase comprises the D33E, T381S, and SN substitutions relative to SEQ ID NO: 3. For example, the recombinant mutant invertase may comprise SEQ ID NO: 27, also referred to as V63 herein.

SEQ ID NO: 27
SMTNETSDRPLVHFTPNKGWMNDPNGLWYDEKEAKWHLYFQYNPNDTVWGTPLFWGHATSDD
LTHWEDEPIAIAPKRNDSGAFSGSMVVDYNNTSGFENDTIDPRQRCVAIWTYNTPESEEQYI
SYSLDGGYTFTEYQKNPVLAANSTQFRDPKVFWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDFGKDYYALQTFFNTDPTYGSALGIAWASNWEYSAFVPTNPWRSSMSLV
RKFSLNTEYQANPETELINLKAEPILNISNAGPWSRFATNTTLTKANSYNVDLSNSTGTLEF
ELVYAVNTSQTISKNVFPDLSLWFKGLEDPEEYLRMGFEASASSFFLDRGNSKVKFVKENPY
FTNRMSVNNQPFKSENDLSYYKVYGLLDQNILELYFNDGDVVSTNTYFMTTGNALGSVNMTT
GVDNLFYIDKFQVREVK.

In certain embodiments, the recombinant mutant invertase comprises the T51Q, P390A, and K425P substitutions relative to SEQ ID NO: 3. For example, the recombinant mutant invertase may comprise SEQ ID NO: 28, also referred to as V70 herein.

SEQ ID NO: 28
SMTNETSDRPLVHFTPNKGWMNDPNGLWYDEKDAKWHLYFQYNPNDTVWGQPLFWGHATSDD
LTHWEDEPIAIAPKRNDSGAFSGSMVVDYNNTSGFENDTIDPRQRCVAIWTYNTPESEEQYI
SYSLDGGYTFTEYQKNPVLAANSTQFRDPKVFWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDFGKDYYALQTFENTDPTYGSALGIAWASNWEYSAFVPTNPWRSSMSLV
RKFSLNTEYQANPETELINLKAEPILNISNAGPWSRFATNTTLTKANSYNVDLSNSTGTLEF
ELVYAVNTTQTISKSVFADLSLWFKGLEDPEEYLRMGFEASASSFFLDRGNSPVKFVKENPY
FTNRMSVNNQPFKSENDLSYYKVYGLLDQNILELYFNDGDVVSTNTYFMTTGNALGSVNMTT
GVDNLFYIDKFQVREVK.

In certain embodiments, the recombinant mutant invertase comprises the F83Y, N340A, and P390A substitutions (relative to SEQ ID NO: 3). For example, the recombinant mutant invertase may comprise SEQ ID NO: 29, also referred to as V78 herein.

SEQ ID NO: 29
SMTNETSDRPLVHFTPNKGWMNDPNGLWYDEKDAKWHLYFQYNPNDTVWGTPLFWGHATSDD
LTHWEDEPIAIAPKRNDSGAYSGSMVVDYNNTSGFFNDTIDPRQRCVAIWTYNTPESEEQYI
SYSLDGGYTFTEYQKNPVLAANSTQFRDPKVFWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDFGKDYYALQTFFNTDPTYGSALGIAWASNWEYSAFVPTNPWRSSMSLV
RKFSLNTEYQANPETELINLKAEPILNISAAGPWSRFATNTTLTKANSYNVDLSNSTGTLEF
ELVYAVNTTQTISKSVFADLSLWFKGLEDPEEYLRMGFEASASSFFLDRGNSKVKFVKENPY
FTNRMSVNNQPFKSENDLSYYKVYGLLDQNILELYENDGDVVSTNTYFMTTGNALGSVNMTT
GVDNLFYIDKFQVREVK.

In certain embodiments, the recombinant mutant invertase comprises the E31A, V89I, and L460I substitutions relative to SEQ ID NO: 3. For example, the recombinant mutant invertase may comprise SEQ ID NO: 30, also referred to as V83 herein.

SEQ ID NO: 30
SMTNETSDRPLVHFTPNKGWMNDPNGLWYDAKDAKWHLYFQYNPNDTVWGTPLFWGHATSDD
LTHWEDEPIAIAPKRNDSGAFSGSMVIDYNNTSGFENDTIDPRQRCVAIWTYNTPESEEQYI
SYSLDGGYTFTEYQKNPVLAANSTQFRDPKVEWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDFGKDYYALQTFFNTDPTYGSALGIAWASNWEYSAFVPTNPWRSSMSLV
RKFSLNTEYQANPETELINLKAEPILNISNAGPWSRFATNTTLTKANSYNVDLSNSTGTLEF
ELVYAVNTTQTISKSVFPDLSLWFKGLEDPEEYLRMGFEASASSFFLDRGNSKVKFVKENPY
FTNRMSVNNQPFKSENDLSYYKVYGILDQNILELYFNDGDVVSTNTYFMTTGNALGSVNMTT
GVDNLFYIDKFQVREVK.

In certain embodiments, the recombinant mutant invertase comprises the E293Q, S448D, and N500K substitutions relative to SEQ ID NO: 3. For example, the recombinant mutant invertase may comprise SEQ ID NO: 31, also referred to as V87 herein.

SEQ ID NO: 31
SMTNETSDRPLVHFTPNKGWMNDPNGLWYDEKDAKWHLYFQYNPNDTVWGTPLFWGHATSDD
LTHWEDEPIAIAPKRNDSGAFSGSMVVDYNNTSGFENDTIDPRQRCVAIWTYNTPESEEQYI
SYSLDGGYTFTEYQKNPVLAANSTQFRDPKVEWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDFGKDYYALQTFFNTDPTYGSALGIAWASNWQYSAFVPTNPWRSSMSLV
RKFSLNTEYQANPETELINLKAEPILNISNAGPWSRFATNTTLTKANSYNVDLSNSTGTLEF
ELVYAVNTTQTISKSVFPDLSLWFKGLEDPEEYLRMGFEASASSFFLDRGNSKVKFVKENPY
FTNRMSVNNQPFKDENDLSYYKVYGLLDQNILELYFNDGDVVSTNTYFMTTGNALGSVNMTT
GVDKLFYIDKFQVREVK.

In certain embodiments, the recombinant mutant invertase comprises the T51L, G497S, and F507I substitutions relative to SEQ ID NO: 3. For example, the recombinant mutant invertase may comprise SEQ ID NO: 32, also referred to as V89 herein.

SEQ ID NO: 32
SMTNETSDRPLVHFTPNKGWMNDPNGLWYDEKDAKWHLYFQYNPNDTVWGLPLFWGHATSDD
LTHWEDEPIAIAPKRNDSGAFSGSMVVDYNNTSGFENDTIDPRQRCVAIWTYNTPESEEQYI
SYSLDGGYTFTEYQKNPVLAANSTQFRDPKVFWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDFGKDYYALQTFENTDPTYGSALGIAWASNWEYSAFVPTNPWRSSMSLV
RKESLNTEYQANPETELINLKAEPILNISNAGPWSRFATNTTLTKANSYNVDLSNSTGTLEF
ELVYAVNTTQTISKSVFPDLSLWEKGLEDPEEYLRMGFEASASSFFLDRGNSKVKFVKENPY
FTNRMSVNNQPFKSENDLSYYKVYGLLDQNILELYFNDGDVVSTNTYFMTTGNALGSVNMTT
SVDNLFYIDKIQVREVK.

In certain embodiments, the recombinant mutant invertase comprises the E293Q, L370F, and K430R substitutions relative to SEQ ID NO: 3. For example, the recombinant mutant invertase may comprise SEQ ID NO: 33, also referred to as V90 herein.

SEQ ID NO: 33
SMTNETSDRPLVHFTPNKGWMNDPNGLWYDEKDAKWHLYFQYNPNDTVWGTPLFWGHATSDD
LTHWEDEPIAIAPKRNDSGAFSGSMVVDYNNTSGFENDTIDPRQRCVAIWTYNTPESEEQYI
SYSLDGGYTFTEYQKNPVLAANSTQFRDPKVEWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDFGKDYYALQTFFNTDPTYGSALGIAWASNWQYSAFVPTNPWRSSMSLV
RKFSLNTEYQANPETELINLKAEPILNISNAGPWSRFATNTTLTKANSYNVDLSNSTGTFEF
ELVYAVNTTQTISKSVFPDLSLWFKGLEDPEEYLRMGFEASASSFFLDRGNSKVKFVRENPY
FTNRMSVNNQPFKSENDLSYYKVYGLLDQNILELYFNDGDVVSTNTYFMTTGNALGSVNMTT
GVDNLFYIDKFQVREVK.

In certain embodiments, the recombinant mutant invertase comprises a non-wild type invertase with F172Y and N429A mutations relative to SEQ ID NO: 2, without signal sequence. For example, the recombinant mutant invertase may comprise the Sc_S288C invertase with F172Y and N429A mutations (SEQ ID NO: 50)

(SEQ ID NO: 50)
SMTNETSDRPLVHFTPNKGWMNDPNGLWYDEKDAKWHLYFQYNPNDTVWGTPLFWGHATSDD
LTNWEDQPIAIAPKRNDSGAYSGSMVVDYNNTSGFENDTIDPRQRCVAIWTYNTPESEEQYI
SYSLDGGYTFTEYQKNPVLAANSTQFRDPKVFWYEPSQKWIMTAAKSQDYKIEIYSSDDLKS
WKLESAFANEGFLGYQYECPGLIEVPTEQDPSKSYWVMFISINPGAPAGGSFNQYFVGSENG
THFEAFDNQSRVVDFGKDYYALQTFENTDPTYGSALGIAWASNWEYSAFVPTNPWRSSMSLV
RKFSLNTEYQANPETELINLKAEPILNISAAGPWSRFATNTTLTKANSYNVDLSNSTGTLEF
ELVYAVNTTQTISKSVFADLSLWFKGLEDPEEYLRMGFEVSASSFFLDRGNSKVKFVKENPY
FTNRMSVNNQPFKSENDLSYYKVYGLLDQNILELYENDGDVVSTNTYFMTTGNALGSVNMTT
GVDNLFYIDKFQVREVK

In certain embodiments, the recombinant mutant invertase comprises the amino acid sequence of any one of SEQ ID NOs: 4-18, or an amino acid sequence that has at least 85%, 90%, 95%, 96%, 97%, 98%, or 99% sequence identity to any one of SEQ ID NOs: 4-18.

In certain embodiments, the recombinant mutant invertase comprises the amino acid sequence of SEQ ID NO: 4, or an amino acid sequence that has at least 85%, 90%, 95%, 96%, 97%, 98%, or 99% sequence identity to SEQ ID NO: 4. In certain embodiments, the recombinant mutant invertase comprises the amino acid sequence of SEQ ID NO: 5, or an amino acid sequence that has at least 85%, 90%, 95%, 96%, 97%, 98%, or 99% sequence identity to SEQ ID NO: 5. In certain embodiments, the recombinant mutant invertase comprises the amino acid sequence of SEQ ID NO: 6, or an amino acid sequence that has at least 85%, 90%, 95%, 96%, 97%, 98%, or 99% sequence identity to SEQ ID NO: 6. In certain embodiments, the recombinant mutant invertase comprises the amino acid sequence of SEQ ID NO: 7, or an amino acid sequence that has at least 85%, 90%, 95%, 96%, 97%, 98%, or 99% sequence identity to SEQ ID NO: 7. In certain embodiments, the recombinant mutant invertase comprises the amino acid sequence of SEQ ID NO: 8, or an amino acid sequence that has at least 85%, 90%, 95%, 96%, 97%, 98%, or 99% sequence identity to SEQ ID NO: 8. In certain embodiments, the recombinant mutant invertase comprises the amino acid sequence of SEQ ID NO: 9, or an amino acid sequence that has at least 85%, 90%, 95%, 96%, 97%, 98%, or 99% sequence identity to SEQ ID NO: 9. In certain embodiments, the recombinant mutant invertase comprises the amino acid sequence of SEQ ID NO: 10, or an amino acid sequence that has at least 85%, 90%, 95%, 96%, 97%, 98%, or 99% sequence identity to SEQ ID NO: 10. In certain embodiments, the recombinant mutant invertase comprises the amino acid sequence of SEQ ID NO: 11, or an amino acid sequence that has at least 85%, 90%, 95%, 96%, 97%, 98%, or 99% sequence identity to SEQ ID NO: 11. In certain embodiments, the recombinant mutant invertase comprises the amino acid sequence of SEQ ID NO: 12, or an amino acid sequence that has at least 85%, 90%, 95%, 96%, 97%, 98%, or 99% sequence identity to SEQ ID NO: 12. In certain embodiments, the recombinant mutant invertase comprises the amino acid sequence of SEQ ID NO: 13, or an amino acid sequence that has at least 85%, 90%, 95%, 96%, 97%, 98%, or 99% sequence identity to SEQ ID NO: 13. In certain embodiments, the recombinant mutant invertase comprises the amino acid sequence of SEQ ID NO: 14, or an amino acid sequence that has at least 85%, 90%, 95%, 96%, 97%, 98%, or 99% sequence identity to SEQ ID NO: 14. In certain embodiments, the recombinant mutant invertase comprises the amino acid sequence of SEQ ID NO: 15, or an amino acid sequence that has at least 85%, 90%, 95%, 96%, 97%, 98%, or 99% sequence identity to SEQ ID NO: 15. In certain embodiments, the recombinant mutant invertase comprises the amino acid sequence of SEQ ID NO: 16, or an amino acid sequence that has at least 85%, 90%, 95%, 96%, 97%, 98%, or 99% sequence identity to SEQ ID NO: 16. In certain embodiments, the recombinant mutant invertase comprises the amino acid sequence of SEQ ID NO: 17, or an amino acid sequence that has at least 85%, 90%, 95%, 96%, 97%, 98%, or 99% sequence identity to SEQ ID NO: 17. In certain embodiments, the recombinant mutant invertase comprises the amino acid sequence of SEQ ID NO: 18 or an amino acid sequence that has at least 85%, 90%, 95%, 96%, 97%, 98%, or 99% sequence identity to SEQ ID NO: 18.

In certain embodiments, the recombinant mutant invertase comprises the amino acid sequence of any one of SEQ ID NOs: 19-33, or an amino acid sequence that has at least 85%, 90%, 95%, 96%, 97%, 98%, or 99% sequence identity to any one of SEQ ID NOs: 19-33.

In certain embodiments, the recombinant mutant invertase comprises the amino acid sequence of SEQ ID NO: 19, or an amino acid sequence that has at least 85%, 90%, 95%, 96%, 97%, 98%, or 99% sequence identity to SEQ ID NO: 19. In certain embodiments, the recombinant mutant invertase comprises the amino acid sequence of SEQ ID NO: 20, or an amino acid sequence that has at least 85%, 90%, 95%, 96%, 97%, 98%, or 99% sequence identity to SEQ ID NO: 20. In certain embodiments, the recombinant mutant invertase comprises the amino acid sequence of SEQ ID NO: 21, or an amino acid sequence that has at least 85%, 90%, 95%, 96%, 97%, 98%, or 99% sequence identity to SEQ ID NO: 21. In certain embodiments, the recombinant mutant invertase comprises the amino acid sequence of SEQ ID NO: 22, or an amino acid sequence that has at least 85%, 90%, 95%, 96%, 97%, 98%, or 99% sequence identity to SEQ ID NO: 22. In certain embodiments, the recombinant mutant invertase comprises the amino acid sequence of SEQ ID NO: 23, or an amino acid sequence that has at least 85%, 90%, 95%, 96%, 97%, 98%, or 99% sequence identity to SEQ ID NO: 23. In certain embodiments, the recombinant mutant invertase comprises the amino acid sequence of SEQ ID NO: 24, or an amino acid sequence that has at least 85%, 90%, 95%, 96%, 97%, 98%, or 99% sequence identity to SEQ ID NO: 24. In certain embodiments, the recombinant mutant invertase comprises the amino acid sequence of SEQ ID NO: 25, or an amino acid sequence that has at least 85%, 90%, 95%, 96%, 97%, 98%, or 99% sequence identity to SEQ ID NO: 25. In certain embodiments, the recombinant mutant invertase comprises the amino acid sequence of SEQ ID NO: 26, or an amino acid sequence that has at least 85%, 90%, 95%, 96%, 97%, 98%, or 99% sequence identity to SEQ ID NO: 26. In certain embodiments, the recombinant mutant invertase comprises the amino acid sequence of SEQ ID NO: 27, or an amino acid sequence that has at least 85%, 90%, 95%, 96%, 97%, 98%, or 99% sequence identity to SEQ ID NO: 27. In certain embodiments, the recombinant mutant invertase comprises the amino acid sequence of SEQ ID NO: 28, or an amino acid sequence that has at least 85%, 90%, 95%, 96%, 97%, 98%, or 99% sequence identity to SEQ ID NO: 28. In certain embodiments, the recombinant mutant invertase comprises the amino acid sequence of SEQ ID NO: 29, or an amino acid sequence that has at least 85%, 90%, 95%, 96%, 97%, 98%, or 99% sequence identity to SEQ ID NO: 29. In certain embodiments, the recombinant mutant invertase comprises the amino acid sequence of SEQ ID NO: 30, or an amino acid sequence that has at least 85%, 90%, 95%, 96%, 97%, 98%, or 99% sequence identity to SEQ ID NO: 30. In certain embodiments, the recombinant mutant invertase comprises the amino acid sequence of SEQ ID NO: 31, or an amino acid sequence that has at least 85%, 90%, 95%, 96%, 97%, 98%, or 99% sequence identity to SEQ ID NO: 31. In certain embodiments, the recombinant mutant invertase comprises the amino acid sequence of SEQ ID NO: 32, or an amino acid sequence that has at least 85%, 90%, 95%, 96%, 97%, 98%, or 99% sequence identity to SEQ ID NO: 32. In certain embodiments, the recombinant mutant invertase comprises the amino acid sequence of SEQ ID NO: 33 or an amino acid sequence that has at least 85%, 90%, 95%, 96%, 97%, 98%, or 99% sequence identity to SEQ ID NO: 33.

As used herein, percent “identity” between a polypeptide sequence and a reference sequence is defined as the percentage of amino acid residues in the polypeptide sequence that are identical to the amino acid residues in the reference sequence, after aligning the sequences and introducing gaps, if necessary, to achieve the maximum percent sequence identity. Similarly, percent “identity” between a nucleic acid sequence and a reference sequence is defined as the percentage of nucleotides in the nucleic acid sequence that are identical to the nucleotides in the reference sequence, after aligning the sequences and introducing gaps, if necessary, to achieve the maximum percent sequence identity. Alignment for purposes of determining percent sequence identity (e.g., amino acid sequence identity or nucleic acid sequence identity) can be achieved in various ways that are within the skill in the art, for instance, using publicly available computer software such as BLAST, BLAST-2, ALIGN, MEGALIGN (DNASTAR), CLUSTALW, CLUSTAL OMEGA, or MUSCLE software. Those skilled in the art can determine appropriate parameters for aligning sequences, including any algorithms needed to achieve maximal alignment over the full length of the sequences being compared.

BLAST (Basic Local Alignment Search Tool) analysis using the algorithm employed by the programs blastp, blastn, blastx, tblastn and tblastx (Karlin et al., (1990) PROC. NATL. ACAD. SCI. USA 87:2264-2268; Altschul, (1993) J. MOL. EVOL. 36, 290-300; Altschul et al., (1997) NUCLEIC ACIDS RES. 25:3389-3402, incorporated by reference) are tailored for sequence similarity searching. For a discussion of basic issues in searching sequence databases, see Altschul et al., (1994) NATURE GENETICS 6:119-129, which is fully incorporated by reference. Those skilled in the art can determine appropriate parameters for measuring alignment, including any algorithms needed to achieve maximal alignment over the full length of the sequences being compared. The search parameters for histogram, descriptions, alignments, expect (i.e., the statistical significance threshold for reporting matches against database sequences), cutoff, matrix and filter are at the default settings. The default scoring matrix used by blastp, blastx, tblastn, and tblastx is the BLOSUM62 matrix (Henikoff et al., (1992) PROC. NATL. ACAD. SCI. USA 89:10915-10919, fully incorporated by reference). Four blastn parameters may be adjusted as follows: Q=10 (gap creation penalty); R=10 (gap extension penalty); wink=1 (generates word hits at every wink.sup.th position along the query); and gapw=16 (sets the window width within which gapped alignments are generated). The equivalent Blastp parameter settings may be Q=9; R=2; wink=1; and gapw=32. Searches may also be conducted using the NCBI (National Center for Biotechnology Information) BLAST Advanced Option parameter (e.g.: -G, Cost to open gap [Integer]: default=5 for nucleotides/11 for proteins; -E, Cost to extend gap [Integer]: default=2 for nucleotides/1 for proteins; -q, Penalty for nucleotide mismatch [Integer]: default=−3; -r, reward for nucleotide match [Integer]: default=1; -e, expect value [Real]: default=10; -W, wordsize [Integer]: default=11 for nucleotides/28 for megablast/3 for proteins; -y, Dropoff (X) for blast extensions in bits: default=20 for blastn/7 for others; −X, X dropoff value for gapped alignment (in bits): default=15 for all programs, not applicable to blastn; and -Z, final X dropoff value for gapped alignment (in bits): 50 for blastn, 25 for others). ClustalW for pairwise protein alignments may also be used (default parameters may include, e.g., Blosum62 matrix and Gap Opening Penalty=10 and Gap Extension Penalty=0.1). A Bestfit comparison between sequences, available in the GCG package version 10.0, uses DNA parameters GAP=50 (gap creation penalty) and LEN=3 (gap extension penalty) and the equivalent settings in protein comparisons are GAP=8 and LEN=2.

In certain embodiments, a recombinant mutant invertase has increased stability at acidic pH (e.g., pH 3.0 or 4.0) relative to a corresponding wild-type invertase enzyme. An increased stability at acidic pH may, in certain conditions, allow the recombinant mutant invertase to survive acidic conditions of the digestive system, especially the stomach. An increased stability at acidic pH may, in certain conditions, also allow the recombinant mutant invertase to survive in certain food or beverages, e.g., fruit juice.

In certain embodiments, the invertase has a specific activity at about pH 3.5 of at least 800, 900, 1,000, 1,100, 1,200, 1,300, 1,400, 1,500, 1,600, 1,700, 1,800, 1,900, or 2,000 μmol sucrose consumed per minute per milligram of deglycosylated invertase. In certain embodiments, the invertase has at least 1 fold, 1.5 fold, 2 fold, 2.5 fold, or 3 fold higher activity at about pH 3.5, compared to a corresponding wild-type invertase. In certain embodiments, the invertase has a specific activity at about pH 5.0 of at least 1,000, 1,100, 1,200, 1,300, 1,400, 1,500, 1,600, 1,700, 1,800, 1,900, 2,000, 2,100, or 2,200 μmol sucrose consumed per minute per milligram of deglycosylated invertase. In certain embodiments, the invertase has at least 1 fold, 1.5 fold, 2 fold, 2.5 fold, or 3 fold higher activity at about pH 5.0, compared to a corresponding wild-type invertase. In certain embodiments, the invertase has a specific activity at about pH 6.0 or 6.2 of at least 700, 800, 900, 1,000, 1,100, 1,200, 1,300, 1,400, 1,500, 1,600, 1,700, or 1,800 μmol sucrose consumed per minute per milligram of deglycosylated invertase. In certain embodiments, the invertase has at least 1 fold, 1.5 fold, 2 fold, 2.5 fold, or 3 fold higher activity at about pH 6.0 or 6.2, compared to a corresponding wild-type invertase. In certain embodiments, the invertase has a specific activity at about pH 7.0 or 7.1 of at least 200, 300, 400, 500, 600, 700, 800, 900, 1,000, 1,100, 1,200, or 1,300 μmol sucrose consumed per minute per milligram of deglycosylated invertase. In certain embodiments, the invertase has at least 1 fold, 1.5 fold, 2 fold, 2.5 fold, or 3 fold higher activity at about pH 7.0 or 7.1, compared to a corresponding wild-type invertase. In certain embodiments, the invertase retains at least 20%, 30%, 40%, 50%, 60%, 70%, 80%, 90%, 95%, 96%, 97%, 98%, or 99% of activity following incubation at about pH 2.5 for about 30 minutes. In certain embodiments, the invertase has at least 1 fold, 1.5 fold, 2 fold, 2.5 fold, 3 fold, 3.5 fold, 4 fold, or 5 fold higher stability at about pH 2.5 compared to a corresponding wild-type invertase.

Methods for testing the stability and activity of an invertase are known in the art and can include, for example, the methods described in Example 1 herein. In certain embodiments, stability of an invertase in low pH is determined using by exposing the invertase to a specific pH, and using the dinitro salicylate (DNS) colorimetric method to monitor reducing sugar formation upon hydrolysis of sucrose.

In certain embodiments, a recombinant mutant invertase has increased stability at higher temperature (e.g., Tm of 56-68° C.) relative to a corresponding wild-type invertase enzyme. In certain embodiments, the invertase has a Tm of at least 56, 57, 58, 59, 60, 61, 62, 63, 64, 65, 66, 67, or 68° C. In certain embodiments the invertase has a Tm that is at least 1, 2, 3, 4, 5, 6, 7, 8, 9, or 10° C. higher than a corresponding wild-type invertase.

Methods for testing the thermostability and activity of an invertase are known in the art and can include, for example, the methods described in Example 1 herein. In certain embodiments, stability and activity of an invertase at high temperature is determined by treatment of invertase samples at high temperature prior to assaying residual activity using the DNS method as described above.

In certain embodiments, the invertase has increased stability in the presence of a protease (e.g., a serine protease and/or an aspartic protease) relative to the corresponding wild-type invertase enzyme. Increased stability in the presence of a protease may, in certain conditions, allow a recombinant mutant invertase to survive the conditions of the stomach. In certain embodiments, a recombinant mutant invertase has increased stability in the presence of pancreatin or pepsin relative to a corresponding wild-type invertase enzyme.

In certain embodiments, a disclosed invertase enzyme retains at least 20%, 30%, 40%, 50%, 60%, 70%, 80%, 90%, 95%, 96%, 97%, 98%, or 99% of activity following incubation with pepsin (e.g., incubation with about 10 mg/ml pepsin at about at 37° C. in simulated gastric fluid (SGF) for about 2 hours). In certain embodiments, a disclosed invertase enzyme retains at least 20%, 30%, 40%, 50%, 60%, 70%, 80%, 90%, 95%, 96%, 97%, 98%, or 99% of activity following incubation with pancreatin (e.g., incubation with about 3.4 mg/ml pancreatin at about at 37° C. in simulated intestinal fluid (SGF) for about 2 hours).

Methods for testing the proteolytic stability and activity of an invertase are known in the art and can include, for example, the methods described in Example 1 herein. In certain embodiments, stability and activity of an invertase in the presence of pepsin or pancreatin is determined by incubation of invertase with pepsin or pancreatin prior to assaying residual activity using the DNS method as described above.

### II. Isomaltase Enzymes

Among other things, the invention provides pharmaceutical compositions optionally further comprising an isomaltase enzyme that, for example, are useful in treating disorders such as congenital sucrase-isomaltase deficiency (CSID).

As used herein, the term “isomaltase” refers to any enzyme, or a functional fragment thereof, that is capable of catalyzing the cleavage of branched (1-6 linked) α-limit dextrins (starches). Isomaltases are also called α-glucosidase, oligo-1,6-glucosidase, and EC 3.2.1.10, and, unless indicated otherwise, the terms are used interchangeably herein. The term isomaltase includes variants having one or more amino acid substitutions, deletions, or insertions relative to a wild-type isomaltase sequence, and/or fusion proteins or conjugates including a isomaltase. As used herein, the term “functional fragment” of a isomaltase refers to fragment of a full-length isomaltase that retains, for example, at least 10%, at least 20%, at least 30%, at least 40%, at least 50%, at least 60%, at least 70%, at least 80%, at least 90%, or 100% of the enzymatic activity of the corresponding full-length, naturally occurring isomaltase. Exemplary isomaltase activity assays are described in Noguchi et al. (2003) J. BIOCHEM. 134 (4): 543-50 and Schonert et al. (1998) J. BACTERIOL. 180 (9): 2574-8. Additionally, because glucose is a product of the enzymatic reactions catalyzed by both invertase and isomaltase, it is understood that an assay based on the detection of glucose may in certain embodiments be used to measure the activity of both an invertase enzyme and an isomaltase enzyme.

Exemplary isomaltase enzymes include isomaltase enzymes derived from Saccharomyces cerevisiae. The amino acid sequences of exemplary wild-type isomaltase enzymes derived from Saccharomyces cerevisiae are depicted in SEQ ID NOs: 37-41 and nucleotide sequences encoding exemplary wild-type isomaltase enzymes derived from Saccharomyces cerevisiae are depicted in SEQ ID NOs: 42-46.

An exemplary wild-type isomaltase enzyme derived from L. fermentum is depicted in SEQ ID NO: 47.

SEQ ID NO: 47
MIYTPKWWWQNSVVYQVYPRSFQDSNHDGIGDLKGIISRLDYIKKLGADI
IWLNPIYRSPNVDNGYDISDYRAIDPTFGSLTDFKELLTKAHELGLKIMM
DLVVNHSSDENEWFKQSRQGKENPYRDYYIWRDPVDGHEPTNWGSYFSGS
AWQYDETSGQYYLHLFAVKQPDLNWENEAVRHSVYDIMNWWADLGVDGFR
MDVINLISKPAVYKDVPTAPGMQYGDVEPVVANGHRMHEFLQEMHQAVMA
KHDLVTVGETPGATTDDAKKYANLEQTELNMVFEFEHVGLDGNDNPALGK
WSDKKVSLPELRDNLVKWQTQLNGKAWNSLYWNNHDQPRVVSREGNDDPK
YRVVSAKMLATMLHCLQGTPYIYAGEELGMTNTTENSLSDYRDLESINAY
HQLVDEEHLVDGKTMSRYLAIHSRDNARTPMQWDDSKNAGFSDAEPWIAV
NPNYSEINAKAALADPSSVFYHYQKLIQMRHDLPVMTEGKFALVNGNELD
EQVFAYTRDDGETTLLVVANFTKETIKREYAAGQGKLLLSNYEDDMGETL
RPYEAKVYEFSSKR

An exemplary isomaltase enzyme variant may comprise an amino acid sequence that has at least 80%, 85%, 90%, 91%, 92%, 93%, 94%, 95%, 96%, 97%, 98%, or 99% sequence identity to SEQ ID NO: 47. In certain embodiments, the isomaltase enzyme variant comprises at least one amino acid substitution (e.g., 1, 2, 3, 4, 5, 6, 7, 8, 9, 10 amino acid substitutions) and an amino acid sequence that has at least 98%, 98.5%, 99% or 99.5% sequence identity to SEQ ID NO: 47. In certain embodiments, the isomaltase comprises substitutions at positions corresponding to the amino acids sequence set forth in SEQ ID NO: 47, including: the E93K, K115I, R132K, D226S, E310A, L366M, I421A, A444G, A531D, and F560L substitutions; the E93K, K115I, R132K, D226S, E310A, I421A, A444G, E524Q, A531D, and F560L substitutions; the E93K, K115I, R132K, D226S, E310A, I421A, A444G, A531D, and F560V substitutions; the E93K, K115I, R132K, D226S, L366M, I421A, A444G, E524Q, A531D, and F560L substitutions; the E93K, K115I, R132K, D226S, L366M, I421A, A444G, A531D, and F560V substitutions; the E93K, K115I, R132K, D226S, I421A, A444G, E524Q, A531D, and F560V substitutions; the K115I, R132K, D226S, E310A, L366M, I421A, A444G, E524Q, A531D, and F560L substitutions; the K115I, R132K, D226S, E310A, L366M, I421A, A444G, A531D, and F560V substitutions; the K115I, R132K, D226S, E310A, I421A, A444G, E524Q, A531D, and F560V substitutions; the K115I, R132K, D226S, L366M, I421A, A444G, E524Q, A531D, and F560V substitutions; the E93K, K115I, R132K, A211E, D226S, I421A, A444G, E524Q, A531D, and F560V substitutions; the E93K, K115I, R132K, D226S, L366M, I421A, A444G, E524Q, A531D, and F560V substitutions; the E93K, K115I, R132K, D226S, E275D, I421A, A444G, E524Q, A531D, and F560V substitutions; the T89A, E93K, K115I, R132K, D226S, I421A, A444G, E524Q, A531D, and F560V substitutions; the E93K, K115I, R132K, H182R, D226S, I421A, A444G, E524Q, A531D, and F560V substitutions; the E93K, K115I, E122D, R132K, D226S, I421A, A444G, E524Q, A531D, and F560V substitutions; the E93K, K115I, D226S, I421A, A444G, E524Q, A531D, and F560V substitutions; the E93K, K115I, D226S, I421A, A444G, E524Q, A531D, and F560L substitutions; the K115I, A211E, D226S, I421A, A444G, A531D, and F560V substitutions; the K115I, D226S, L366M, I421A, A444G, E524Q, A531D, and F560L substitutions; the K115I, A211E, D226S, I421A, A444G, E524Q, A531D, and F560L substitutions; the K115I, R132K, D226S, E310A, L366M, I421A, A444G, A531D, and F560L substitutions; the E93K, K115I, D226S, E310A, 1421A, A444G, A531D, and F560L substitutions; the K115I, D226S, E310A, I421A, A444G, A531D, and F560V substitutions; the E93K, K115I, D226S, L366M, 1421A, A444G, A531D, and F560L substitutions; the K115I, D226S, 1421A, A444G, E524Q, A531D, and F560V substitutions; the K115I, D226S, E310A, 1421A, A444G, E524Q, A531D, and F560L substitutions; the K115I, A211E, D226S, L366M, 1421A, A444G, A531D, and F560L substitutions; the K115I, A211E, D226S, E310A, I421A, A444G, A531D, and F560L substitutions; the E93K, K115I, D226S, I421A, A444G, A531D, and F560V substitutions; the E93K, K115I, A211E, D226S, I421A, A444G, A531D, and F560L substitutions; or the K115I, D226S, L366M, I421A, A444G, A531D, and F560V substitutions.

Additional exemplary isomaltase enzymes can be found on the world wide web at brenda-enzymes.org/enzyme.php?ecno=3.2.1.10&onlyTable=Sequence.

A contemplated isomaltase enzyme may comprise the amino acid sequence of any one of SEQ ID NOs: 37-41, or an amino acid sequence that has at least 80%, 85%, 90%, 95%, 96%, 97%, 98%, or 99% sequence identity to any one of SEQ ID NOs: 42-46. Other isomaltase enzymes that may be used in combination with the recombinant invertase enzymes described herein are described in U.S. Provisional Patent Application No. 63/388,845, filed on Jul. 13, 2022.

In certain embodiments, the isomaltase enzyme comprises one or more conservative substitutions relative to isomaltase enzyme disclosed herein. In other embodiments, the isomaltase enzyme comprises one or more non-conservative substitutions relative to isomaltase enzyme disclosed herein.

In certain embodiments, the composition comprises (i) an invertase comprising the amino acid sequence of SEQ ID NO: 14 (variant V78 with a FAKS signal sequence) or SEQ ID NO: 29 (variant 78 without a signal sequence) and (ii) one or more of the isomaltase enzymes described in this Section II.

In certain embodiments, the composition comprises (i) an invertase comprising the amino acid sequence of SEQ ID NO: 49 (Sc_S288C invertase with F172Y and N429A mutations relative to SEQ ID NO: 2, with wild type signal sequence) or SEQ ID NO: 50 (Sc_S288C invertase with F172Y and N429A mutations relative to SEQ ID NO: 2 without a signal sequence) and (ii) one or more of the isomaltase enzymes described in this Section II.

### III. Enzyme Production

Methods for producing invertase and/or isomaltase enzymes are known in the art. For example, DNA molecules encoding an invertase and/or isomaltase enzyme can be chemically synthesized using the sequence information provided herein. Synthetic DNA molecules can be ligated to other appropriate nucleotide sequences, including, e.g., expression control sequences, to produce conventional gene expression constructs encoding the desired invertase and/or isomaltase enzyme.

Nucleic acids encoding desired invertase and/or isomaltase enzymes can be incorporated (ligated) into expression vectors, which can be introduced into host cells through conventional transfection or transformation techniques. Transformed host cells can be grown under conditions that permit the host cells to express the genes that encode the invertase and/or isomaltase enzyme.

An exemplary DNA sequence encoding a wild-type S. cerevisiae invertase is depicted in SEQ ID NO: 35.

(wild type S. cerevisiae invertase DNA sequence):
SEQ ID NO: 35
ATGCTTTTGCAAGCTTTCCTTTTCCTTTTGGCTGGTTTTGCAGCCAAAATATCTGCATCAAT
GACAAACGAAACTAGCGATAGACCTTTGGTCCACTTCACACCCAACAAGGGCTGGATGAATG
ACCCAAATGGGTTGTGGTACGATGAAAAAGATGCCAAATGGCATCTGTACTTTCAATACAAC
CCAAATGACACCGTATGGGGTACGCCATTGTTTTGGGGCCATGCTACTTCCGATGATTTGAC
TCATTGGGAAGATGAACCCATTGCTATCGCTCCCAAGCGTAACGATTCAGGTGCTTTCTCTG
GCTCCATGGTGGTTGATTACAACAACACGAGTGGGTTTTTCAATGATACTATTGATCCAAGA
CAAAGATGCGTTGCGATTTGGACTTATAACACTCCTGAAAGTGAAGAGCAATACATTAGCTA
TTCTCTTGATGGTGGTTACACTTTTACTGAATACCAAAAGAACCCTGTTTTAGCTGCCAACT
CCACTCAATTCAGAGATCCAAAGGTGTTCTGGTATGAACCTTCTCAAAAATGGATTATGACG
GCTGCCAAATCACAAGACTACAAAATTGAAATTTACTCCTCTGATGACTTGAAGTCCTGGAA
GCTAGAATCTGCATTTGCTAATGAAGGTTTCTTAGGCTACCAATATGAATGTCCAGGTTTGA
TTGAAGTCCCAACTGAGCAAGATCCTTCCAAATCCTATTGGGTCATGTTTATTTCTATCAAT
CCAGGTGCACCTGCTGGCGGTTCCTTCAACCAATATTTTGTTGGATCCTTCAATGGTACTCA
TTTTGAAGCGTTTGACAATCAATCTAGAGTGGTAGATTTTGGTAAGGACTACTATGCCTTGC
AAACTTTCTTCAACACAGACCCAACGTACGGTTCAGCATTAGGTATTGCCTGGGCTTCAAAC
TGGGAGTACAGTGCCTTTGTCCCAACTAACCCATGGAGATCATCCATGTCTTTGGTCCGCAA
GTTTTCTTTGAACACTGAATATCAAGCTAATCCAGAGACTGAATTGATCAATTTGAAAGCCG
AACCAATATTGAACATTAGTAATGCTGGTCCCTGGTCTCGTTTTGCTACTAACACAACTCTA
ACTAAGGCCAATTCTTACAATGTCGATTTGAGCAACTCGACTGGTACCCTAGAGTTTGAGTT
GGTTTACGCTGTTAACACCACACAAACCATATCCAAATCCGTCTTTCCCGACTTATCACTTT
GGTTCAAGGGTTTAGAAGATCCTGAAGAATATTTAAGAATGGGTTTTGAAGCCAGTGCTTCT
TCCTTCTTTTTGGACCGTGGTAACTCTAAGGTCAAGTTTGTCAAGGAGAACCCATATTTCAC
AAACAGAATGTCTGTCAACAACCAACCATTCAAGTCTGAGAACGACCTAAGTTACTATAAAG
TGTACGGCCTACTGGATCAAAACATCTTGGAATTGTACTTCAACGATGGAGATGTGGTTTCT
ACAAATACCTACTTCATGACCACCGGTAACGCTCTAGGATCTGTGAACATGACCACTGGTGT
CGATAATTTGTTCTACATTGACAAGTTCCAAGTAAGGGAAGTAAAATAG

An exemplary DNA sequence encoding a wild-type L. fermentum isomaltase is provided in SEQ ID NO: 48.

(wild type L. fermentum isomaltase DNA sequence):
SEQ ID NO: 48
ATGATATATACACCAAAATGGTGGTGGCAAAATTCAGTTGTCTACCAAGTCTATCCACGGAG
TTTTCAAGACAGCAATCATGATGGCATTGGTGATTTAAAAGGAATCATCAGTCGGCTTGACT
ATATTAAAAAACTAGGTGCTGATATTATCTGGCTCAATCCAATCTACCGTTCGCCAAACGTT
GATAATGGGTATGACATCAGTGATTATCGGGCAATTGATCCAACTTTTGGCTCATTGACCGA
CTTTAAGGAGCTGCTGACAAAGGCTCATGAACTTGGATTAAAAATAATGATGGATCTGGTGG
TCAATCACTCATCAGATGAAAATGAATGGTTTAAGCAAAGCCGCCAAGGAAAAGAAAATCCA
TACCGTGACTATTATATCTGGCGTGATCCAGTCGATGGCCATGAACCAACTAATTGGGGATC
ATACTTCTCTGGTTCAGCTTGGCAGTACGATGAGACAAGCGGTCAATACTACCTTCACCTGT
TCGCTGTAAAGCAGCCGGATCTAAACTGGGAAAATGAAGCCGTTCGTCATTCGGTATACGAC
ATCATGAATTGGTGGGCAGATCTCGGTGTCGATGGATTTCGAATGGATGTCATCAACTTAAT
TTCAAAACCGGCTGTCTACAAAGACGTTCCAACCGCGCCCGGAATGCAGTATGGCGATGTCG
AGCCGGTTGTTGCCAATGGTCATCGGATGCACGAATTCCTTCAAGAAATGCATCAAGCTGTA
ATGGCCAAGCATGATTTGGTAACAGTTGGCGAAACACCTGGTGCTACAACAGATGATGCCAA
GAAGTACGCCAACCTTGAACAGACTGAGCTGAACATGGTTTTTGAGTTTGAGCATGTCGGCT
TAGACGGTAACGACAACCCAGCTTTAGGAAAATGGAGCGACAAAAAAGTCAGTCTGCCAGAA
TTGCGAGATAACCTGGTGAAATGGCAGACTCAGCTGAATGGTAAGGCTTGGAACTCGCTTTA
CTGGAACAACCATGATCAGCCACGCGTCGTATCCCGTTTTGGCAATGATGATCCAAAATATC
GTGTCGTTTCGGCTAAAATGTTGGCAACAATGCTGCACTGTCTGCAAGGCACGCCTTATATC
TACGCAGGTGAGGAACTGGGAATGACCAACACGACCTTTAACTCTTTGTCTGACTATCGTGA
TTTGGAGAGCATTAATGCCTACCATCAGCTGGTTGACGAAGAGCATCTAGTTGATGGAAAGA
CAATGAGCAGATATCTGGCAATTCATTCTCGGGACAATGCCAGAACGCCAATGCAATGGGAT
GACAGCAAGAATGCCGGATTTTCGGATGCTGAACCTTGGATCGCGGTCAATCCCAACTATTC
AGAGATCAACGCAAAGGCGGCACTGGCGGATCCATCATCCGTCTTCTATCACTACCAGAAAC
TCATTCAAATGCGCCATGACTTGCCAGTAATGACAGAAGGAAAGTTTGCGTTGGTCAATGGC
AACGAATTGGATGAGCAGGTCTTTGCTTACACGCGTGATGATGGAGAAACAACGCTGCTTGT
AGTAGCCAACTTTACTAAGGAAACAATCAAGCGAGAATACGCGGCTGGTCAAGGCAAGCTCT
TATTAAGCAACTACGAGGATGACATGGGAGAAACTCTGCGTCCATATGAAGCTAAAGTATAT
GAATTCAGTTCAAAGAGGTAA

Nucleic acids encoding a recombinant mutant invertase and/or isomaltase may be generated by mutating a nucleotide sequence encoding a wild type invertase, e.g., SEQ ID NO: 1, SEQ ID NO: 2, or SEQ ID NO: 3, or a wild type isomaltase, e.g., SEQ ID NOs: 37-41, using methods known in the art. SEQ ID Nos: 1 and 2 represent isomaltase protein sequences containing N-terminal signal sequences that can be removed post-translationally. SEQ ID NO: 3 represents a corresponding mature isomaltase protein without an —N-terminal signal sequence. Furthermore, nucleic acids encoding such recombinant invertase and/or isomaltase enzymes may be codon optimized for expression in a heterologous cell, e.g., an Escherichia coli, Saccharomyces cerevisiae, or Pichia pastoris cell, using methods known in the art

In one embodiment, the disclosure relates to a nucleotide sequence encoding a recombinant mutant invertase that comprises the following substitutions: E120A, D122E, and P479A, relative to SEQ ID NO:2, e.g., a recombinant mutant invertase referred to as V6 herein.

In one embodiment, the disclosure relates to a nucleotide sequence encoding a recombinant mutant invertase that comprises the following substitutions: T140L. E156Q, and L549I relative to SEQ ID NO: 2, e.g., a recombinant mutant invertase referred to as V9 herein.

In one embodiment, the disclosure relates to a nucleotide sequence encoding a recombinant mutant invertase that comprises the following substitutions: S476A, P479A, and Q597E relative to SEQ ID NO: 2, e.g., a recombinant mutant invertase referred to as V14 herein.

In one embodiment, the disclosure relates to a nucleotide sequence encoding a recombinant mutant invertase that comprises the following substitutions: K259L, S476N, and K514P relative to SEQ ID NO: 2, e.g., a recombinant mutant invertase referred to as V22 herein.

In one embodiment, the disclosure relates to a nucleotide sequence encoding a recombinant mutant invertase that comprises the following substitutions: K277T, E415S, and V581I relative to SEQ ID NO: 2, e.g., a recombinant mutant invertase referred to as V25 herein.

In one embodiment, the disclosure relates to a nucleotide sequence encoding a recombinant mutant invertase that comprises the following substitutions: A123G, T140L. and F143Y relative to SEQ ID NO: 2, e.g., a recombinant mutant invertase referred to as V32 herein.

In one embodiment, the disclosure relates to a nucleotide sequence encoding a recombinant mutant invertase that comprises the following substitutions: N390D, S476N, and K519R relative to SEQ ID NO: 2, e.g., a recombinant mutant invertase referred to as V38 herein.

In one embodiment, the disclosure relates to a nucleotide sequence encoding a recombinant mutant invertase that comprises the following substitutions: E382Q, T573S, and V581I relative to SEQ ID NO: 2, e.g., a recombinant mutant invertase referred to as V60 herein.

In one embodiment, the disclosure relates to a nucleotide sequence encoding a recombinant mutant invertase that comprises the following substitutions: D122E, T470S, and S476N relative to SEQ ID NO: 2, e.g., a recombinant mutant invertase referred to as V63 herein.

In one embodiment, the disclosure relates to a nucleotide sequence encoding a recombinant mutant invertase that comprises the following substitutions: T140Q, P479A, and K514P relative to SEQ ID NO: 2, e.g., a recombinant mutant invertase referred to as V70 herein.

In one embodiment, the disclosure relates to a nucleotide sequence encoding a recombinant mutant invertase that comprises the following substitutions: F172Y, N429A, and P479A relative to SEQ ID NO: 2, e.g., a recombinant mutant invertase referred to as V78 herein.

In one embodiment, the disclosure relates to a nucleotide sequence encoding a recombinant mutant invertase that comprises the following substitutions: E120A, V178I, and L549I relative to SEQ ID NO: 2, e.g., a recombinant mutant invertase referred to as V83 herein.

In one embodiment, the disclosure relates to a nucleotide sequence encoding a recombinant mutant invertase that comprises the following substitutions: E382Q, S537D, and N589K relative to SEQ ID NO: 2, e.g., a recombinant mutant invertase referred to as V87 herein.

In one embodiment, the disclosure relates to a nucleotide sequence encoding a recombinant mutant invertase that comprises the following substitutions: T140L, G586S, and F596I relative to SEQ ID NO: 2, e.g., a recombinant mutant invertase referred to as V89 herein.

In one embodiment, the disclosure relates to a nucleotide sequence encoding a recombinant mutant invertase that comprises the following substitutions: E382Q, L459F, and K519R relative to SEQ ID NO: 2, e.g., a recombinant mutant invertase referred to as V90 herein.

Specific expression and purification conditions will vary depending upon the expression system employed. For example, if a gene is to be expressed in E. coli, S. cerevisiae, or P. pastoris, it can be cloned into an expression vector by positioning the engineered gene downstream from a suitable bacterial or yeast promoter, e.g., Trp or Tac, and a prokaryotic or eukaryotic signal sequence. In yeast, it may be advantageous to introduce the coding gene into the yeast genome (such as by homologous recombination) for higher expression. The expressed protein accumulates in refractile or inclusion bodies or is secreted, and can be harvested after disruption of the cells by French press or sonication, or by simply collecting the supernatant if secreted. The refractile bodies then are solubilized, and the proteins refolded and cleaved by methods known in the art. Secreted proteins can be further purified using methods known in the art such as ion exchange, affinity chromatography, or salt precipitation.

An invertase and/or isomaltase enzyme can be produced by growing (culturing) a host cell transfected with an expression vector encoding such invertase and/or isomaltase enzyme, under conditions that permit expression of the invertase and/or isomaltase enzyme. Following expression, the invertase and/or isomaltase enzyme can be harvested and purified or isolated using techniques known in the art, e.g., affinity tags such as glutathione-S-transferase (GST) and histidine tags.

Exemplary methods for recombinant expression and purification of invertase enzymes are described in Mohandesi et al. (2016) BIOTECH 6 (2): 129. Exemplary methods for recombinant expression and purification of isomaltase enzymes are described in Deng et al. (2014) FEBS OPEN BIO 4:200-212.

In certain embodiments, an invertase and/or isomaltase enzyme is dried, e.g., spray-dried. Pharmaceutical proteins may be dried in many ways, e.g., by removal of water, organic solvent or liquid polymer by means including drying with N₂, air or inert gases, vacuum oven drying, lyophilization, washing with a volatile organic solvent followed by evaporation of the solvent, evaporation in a fume hood, tray drying, fluid bed drying, spray drying, vacuum drying, or roller drying.

Spray drying invertase and/or isomaltase enzymes allows water to be separated from the invertase and/or isomaltase enzyme preparation, allowing for continuous production of dry solids in powder, granulate, or agglomerate form from liquid feedstocks such as emulsions and pumpable suspensions. Spray drying involves the atomization of a liquid feedstock comprising invertase and/or isomaltase enzyme into a spray of droplets and contacting the droplets with hot air or gas in a drying chamber. The atomization process may be conducted using a two-fluid atomizer that mixes the liquid feedstock with a drying gas such as compressed air or nitrogen. Operating conditions and dryer design are selected according to the drying characteristics of the invertase and/or isomaltase enzyme and the desired powder qualities. Exemplary methods for spray drying enzymes are described in United States Patent Application Publication No. 2015/0353913. The invertase and isomaltase enzyme may be spray dried separately or combined and spray dried together.

It is contemplated that a disclosed invertase and/or isomaltase enzyme may be modified, engineered or chemically conjugated. For example, it is contemplated that a disclosed invertase and/or isomaltase enzyme can be conjugated to an effector agent using standard in vitro conjugation chemistries. If the effector agent is a polypeptide, the invertase and/or isomaltase enzyme can be chemically conjugated to the effector or joined to the effector as a fusion protein. Construction of fusion proteins is within ordinary skill in the art.

In certain embodiments, depending upon a particular mode of administration or site of activity, a disclosed invertase and/or isomaltase enzyme can be modified with a moiety that improves its stabilization and/or retention in circulation, e.g., in blood, serum, or other tissues. For example, a disclosed invertase and/or isomaltase enzyme may be conjugated to a polymer, e.g., a substantially non-antigenic polymer, such as a polyalkylene oxide or a polyethylene oxide. In certain embodiments, a disclosed invertase and/or isomaltase enzyme is conjugated to a water soluble polymer, e.g., a hydrophilic polyvinyl polymer, e.g., polyvinylalcohol or polyvinylpyrrolidone. Examples of such polymers include polyalkylene oxide homopolymers such as polyethylene glycol (PEG) or polypropylene glycols, polyoxyethylenated polyols, copolymers thereof and block copolymers thereof. Additional useful polymers include polyoxyalkylenes such as polyoxyethylene, polyoxypropylene, and block copolymers of polyoxyethylene and polyoxypropylene, polymethacrylates, carbomers, and branched or unbranched polysaccharides.

### IV. Pharmaceutical Compositions

For therapeutic use, an invertase and/or isomaltase enzyme described herein preferably is combined with a pharmaceutically acceptable carrier and/or an excipient. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients suitable for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington's Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In another aspect, the disclosure relates to a pharmaceutical composition comprising: an invertase enzyme, e.g., a recombinant mutant invertase as described herein; a separate isomaltase enzyme; and a pharmaceutically acceptable carrier and/or an excipient. In certain embodiments, the invertase and/or the isomaltase is spray-dried.

In certain embodiments of the pharmaceutical compositions disclosed herein (i) the invertase is a microbial invertase (e.g., derived from Saccharomyces cerevisiae), or a functional fragment or variant thereof, (ii) the invertase comprises a sequence of any one of SEQ ID NOs: 1-33, or a functional fragment or variant thereof, (iii) the invertase is a recombinant mutant S. cerevisiae invertase as described herein, (iv) the isomaltase is a microbial isomaltase (e.g., derived from S. cerevisiae or L. fermentum, or a functional fragment or variant of either of the foregoing, (v) the isomaltase comprises any one of SEQ ID NOs: 37-41, or a functional fragment or variant thereof, (vi) the isomaltase comprises a sequence of SEQ ID NO: 47, (vii) the isomaltase comprises substitutions at positions corresponding to the amino acids sequence set forth in SEQ ID NO: 47, including: the E93K, K115I, R132K, D226S, E310A, L366M, I421A, A444G, A531D, and F560L substitutions; the E93K, K115I, R132K, D226S, E310A, I421A, A444G, E524Q, A531D, and F560L substitutions; the E93K, K115I, R132K, D226S, E310A, 1421A, A444G, A531D, and F560V substitutions; the E93K, K115I, R132K, D226S, L366M, I421A, A444G, E524Q, A531D, and F560L substitutions; the E93K, K115I, R132K, D226S, L366M, 1421A, A444G, A531D, and F560V substitutions; the E93K, K115I, R132K, D226S, I421A, A444G, E524Q, A531D, and F560V substitutions; the K115I, R132K, D226S, E310A, L366M, I421A, A444G, E524Q, A531D, and F560L substitutions; the K115I, R132K, D226S, E310A, L366M, I421A, A444G, A531D, and F560V substitutions; the K115I, R132K, D226S, E310A, I421A, A444G, E524Q, A531D, and F560V substitutions; the K115I, R132K, D226S, L366M, I421A, A444G, E524Q, A531D, and F560V substitutions; the E93K, K115I, R132K, A211E, D226S, I421A, A444G, E524Q, A531D, and F560V substitutions; the E93K, K115I, R132K, D226S, L366M, 1421A, A444G, E524Q, A531D, and F560V substitutions; the E93K, K115I, R132K, D226S, E275D, I421A, A444G, E524Q, A531D, and F560V substitutions; the T89A, E93K, K115I, R132K, D226S, I421A, A444G, E524Q, A531D, and F560V substitutions; the E93K, K115I, R132K, H182R, D226S, I421A, A444G, E524Q, A531D, and F560V substitutions; the E93K, K115I, E122D, R132K, D226S, I421A, A444G, E524Q, A531D, and F560V substitutions; the E93K, K115I, D226S, 1421A, A444G, E524Q, A531D, and F560V substitutions; the E93K, K115I, D226S, I421A, A444G, E524Q, A531D, and F560L substitutions; the K115I, A211E, D226S, I421A, A444G, A531D, and F560V substitutions; the K115I, D226S, L366M, I421A, A444G, E524Q, A531D, and F560L substitutions; the K115I, A211E, D226S, 1421A, A444G, E524Q, A531D, and F560L substitutions; the K115I, R132K, D226S, E310A, L366M, I421A, A444G, A531D, and F560L substitutions; the E93K, K115I, D226S, E310A, I421A, A444G, A531D, and F560L substitutions; the K115I, D226S, E310A, I421A, A444G, A531D, and F560V substitutions; the E93K, K115I, D226S, L366M, I421A, A444G, A531D, and F560L substitutions; the K115I, D226S, I421A, A444G, E524Q, A531D, and F560V substitutions; the K115I, D226S, E310A, I421A, A444G, E524Q, A531D, and F560L substitutions; the K115I, A211E, D226S, L366M, I421A, A444G, A531D, and F560L substitutions; the K115I, A211E, D226S, E310A, I421A, A444G, A531D, and F560L substitutions; the E93K, K115I, D226S, I421A, A444G, A531D, and F560V substitutions; the E93K, K115I, A211E, D226S, I421A, A444G, A531D, and F560L substitutions; or the K115I, D226S, L366M, I421A, A444G, A531D, and F560V substitutions, (viii) the isomaltase is a recombinant mutant isomaltase set forth in U.S. Provisional Application No. 63/388,845, filed on Jul. 13, 2022, or (ix) or a combination of any one of features (i)-(viii). In certain embodiments, the invertase comprises the amino acid sequence of SEQ ID NO: 14 or 29, or a functional fragment thereof. In certain embodiments, the invertase comprises the amino acid sequence of or SEQ ID NO: 49 or 50, or a functional fragment thereof.

In certain embodiments, the composition is formulated as an oral dosage form. In certain embodiments, the composition is formulated as a powder, satchel, granulate, pellet, micropellet, tablet, or minitablet. In certain embodiments, the composition is formulated as a powder, satchel, or tablet. In certain embodiments, the composition has a shelf-life at room temperature of at least 3 months, 6 months, 9 months, 12 months, 15 months, 18 months, 21 months, 24 months, 36 months, 48 months, 60 months, 72 months, 84 months, 96 months, 108 months, or 120 months.

In certain embodiments, the invertase and/or isomaltase enzymes can be formulated, or co-administered (either at the same time or sequentially), for example, by an enteral route (e.g., orally), with a pH increasing agent, for example, a protein pump inhibitor (PPI), to enhance the stability of the invertase and/or isomaltase enzyme, for example, in an acidic environment, for example, in the gastrointestinal tract.

Proton pump inhibitors are a group of drugs whose main action is pronounced and long-lasting reduction of gastric acid production. Proton pump inhibitors act by blocking the hydrogen/potassium adenosine triphosphatase enzyme system (the H⁺/K⁺ ATPase, or more commonly just gastric proton pump) of the gastric parietal cell. The proton pump is the terminal stage in gastric acid secretion, being directly responsible for secreting H⁺ ions into the gastric lumen, making it an ideal target for inhibiting acid secretion. Examples of proton pump inhibitors include: Omeprazole (brand names: LOSEC®, PRILOSEC®, ZEGERID®); Lansoprazole (brand names: PREVACID®, ZOTON®, INHIBITOL®); Esomeprazole (brand names: NEXIUM®); and Pantoprazole (brand names: PROTONIX®, SOMAC®, PANTOLOC®).

Pharmaceutical compositions containing a recombinant invertase and/or isomaltase enzyme disclosed herein can be presented in a dosage unit form and can be prepared by any suitable method. A pharmaceutical composition should be formulated to be compatible with its intended route of administration. The pharmaceutical compositions may be in a variety of forms. These include, for example, liquid, semi-solid and solid dosage forms, such as liquid solutions, dispersions or suspensions, tablets, pills, powders, liposomes and suppositories. The preferred form will depend upon the intended mode of administration and therapeutic application. In certain embodiments, the composition is formulated as an oral dosage form. The oral dosage form may, for example, be formulated as a powder, satchel, granulate, pellet, micropellet, tablet, or minitablet. In some embodiments, the composition is formulated as a powder, satchel, or tablet.

Although the compositions preferably are formulated for administration enterally (for example, orally), such compositions can be administered by a parenteral mode (e.g., intravenous, subcutaneous, intraperitoneal, or intramuscular injection). The phrases “parenteral administration” and “administered parenterally” as used herein mean modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and infrasternal injection and infusion.

The composition can be formulated as a solution, microemulsion, dispersion, liposome, or other ordered structure suitable for stable storage at high concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. In the case of sterile powders for the preparation of sterile injectable solutions, the preferred methods of preparation are vacuum drying and freeze drying that yield a powder of an agent described herein plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Depending upon the mode of administration, for example, by parenteral administration, it may be desirable to produce a pharmaceutical formulation that is sterile. Sterilization can be accomplished by any suitable method, e.g., filtration through sterile filtration membranes. Where the composition is lyophilized, filter sterilization can be conducted prior to or following lyophilization and reconstitution.

In certain embodiments, a disclosed composition comprises a polyionic reagent which may, e.g., coat the invertase and/or isomaltase enzyme (e.g., the composition comprises a polyionic coating). Exemplary polyionic reagents include PSS (poly(Sodium 4-styrenesulfonate), PAA (poly Acrylic acid sodium salt), PMG (poly(methylene-co-guanidine) hydrochloride), DS (dextran sulfate), PMA (poly(methyl acrylate)), or PVS (polyvinylsiloxane).

In certain embodiments, a disclosed composition and/or dosage comprises: (i) about 750 to about 75,000, about 750 to about 60,000, about 750 to about 45,000, about 750 to about 30,000, about 750 to about 15,000, about 750 to about 10,000, about 750 to about 7500, about 750 to about 5,000, about 750 to about 2500, about 750 to about 1,000, about 1,000 to about 75,000, about 1,000 to about 60,000, about 1,000 to about 45,000, about 1,000 to about 30,000, about 1,000 to about 15,000, about 1,000 to about 10,000, about 1,000 to about 7,500, about 1,000 to about 5,000, about 1,000 to about 2,500, about 2,500 to about 75,000, about 2,500 to about 60,000, about 2,500 to about 45,000, about 2,500 to about 30,000, about 2,500 to about 15,000, about 2,500 to about 10,000, about 2,500 to about 7,500, about 2,500 to about 5,000, about 5,000 to about 75,000, about 5,000 to about 60,000, about 5,000 to about 45,000, about 5,000 to about 30,000, about 5,000 to about 15,000, about 5,000 to about 10,000, about 5,000 to about 7,500, about 7,500 to about 75,000, about 7,500 to about 60,000, about 7,500 to about 45,000, about 7,500 to about 30,000, about 7,500 to about 15,000, about 7,500 to about 10,000, about 10,000 to about 75,000, about 10,000 to about 60,000, about 10,000 to about 45,000, about 10,000 to about 30,000, about 10,000 to about 15,000, about 15,000 to about 75,000, about 15,000 to about 60,000, about 15,000 to about 45,000, about 15,000 to about 30,000, about 30,000 to about 75,000, about 30,000 to about 60,000, about 30,000 to about 45,000, about 45,000 to about 75,000, about 45,000 to about 60,000, or about 60,000 to about 75,000 international units (I.U.) of invertase enzyme; and/or (ii) about 750 to about 75,000, about 750 to about 60,000, about 750 to about 45,000, about 750 to about 30,000, about 750 to about 15,000, about 750 to about 10,000, about 750 to about 7,500, about 750 to about 5,000, about 750 to about 2,500, about 750 to about 1,000, about 1,000 to about 75,000, about 1,000 to about 60,000, about 1,000 to about 45,000, about 1,000 to about 30,000, about 1,000 to about 15,000, about 1,000 to about 10,000, about 1,000 to about 7,500, about 1,000 to about 5,000, about 1,000 to about 2,500, about 2,500 to about 75,000, about 2,500 to about 60,000, about 2,500 to about 45,000, about 2,500 to about 30,000, about 2,500 to about 15,000, about 2,500 to about 10,000, about 2,500 to about 7,500, about 2,500 to about 5,000, about 5,000 to about 75,000, about 5,000 to about 60,000, about 5,000 to about 45,000, about 5,000 to about 30,000, about 5,000 to about 15,000, about 5,000 to about 10,000, about 5,000 to about 7,500, about 7,500 to about 75,000, about 7,500 to about 60,000, about 7,500 to about 45,000, about 7,500 to about 30,000, about 7,500 to about 15,000, about 7,500 to about 10,000, about 10,000 to about 75,000, about 10,000 to about 60,000, about 10,000 to about 45,000, about 10,000 to about 30,000, about 10,000 to about 15,000, about 15,000 to about 75,000, about 15,000 to about 60,000, about 15,000 to about 45,000, about 15,000 to about 30,000, about 30,000 to about 75,000, about 30,000 to about 60,000, about 30,000 to about 45,000, about 45,000 to about 75,000, about 45,000 to about 60,000, or about 60,000 to about 75,000 international units (I.U.) of isomaltase enzyme.

In certain embodiments, a disclosed composition and/or dosage comprises: (i) about 5,000 to about 12,000, about 5,000 to about 11,000, about 5,000 to about 10,000, about 5,000 to about 9,000, about 5,000 to about 8,000, about 5,000 to about 7,000, about 5,000 to about 6,000, about 6,000 to about 12,000, about 6,000 to about 11,000, about 6,000 to about 10,000, about 6,000 to about 9,000, about 6,000 to about 8,000, about 6,000 to about 7,000, about 7,000 to about 12,000, about 7,000 to about 11,000, about 7,000 to about 10,000, about 7,000 to about 9,000, about 7,000 to about 8,000, about 8,000 to about 12,000, about 8,000 to about 11,000, about 8,000 to about 10,000, about 8,000 to about 9,000, about 9,000 to about 12,000, about 9,000 to about 11,000, about 9,000 to about 10,000, about 10,000 to about 12,000, about 10,000 to about 11,000, about 11,000 to about 12,000, about 5,000, about 6,000, about 7,000, about 8,000, about 8,500, about 9,000, about 10,000, about 11,000, or about 12,000 international units (I.U.) of invertase enzyme; and/or (ii) about 5,000 to about 12,000, about 5,000 to about 11,000, about 5,000 to about 10,000, about 5,000 to about 9,000, about 5,000 to about 8,000, about 5,000 to about 7,000, about 5,000 to about 6,000, about 6,000 to about 12,000, about 6,000 to about 11,000, about 6,000 to about 10,000, about 6,000 to about 9,000, about 6,000 to about 8,000, about 6,000 to about 7,000, about 7,000 to about 12,000, about 7,000 to about 11,000, about 7,000 to about 10,000, about 7,000 to about 9,000, about 7,000 to about 8,000, about 8,000 to about 12,000, about 8,000 to about 11,000, about 8,000 to about 10,000, about 8,000 to about 9,000, about 9,000 to about 12,000, about 9,000 to about 11,000, about 9,000 to about 10,000, about 10,000 to about 12,000, about 10,000 to about 11,000, about 11,000 to about 12,000, about 5,000, about 6,000, about 7,000, about 8,000, about 8,500, about 9,000, about 10,000, about 11,000, or about 12,000 international units (I.U.) of isomaltase enzyme.

In certain embodiments, a disclosed invertase and/or isomaltase enzyme or composition is administered to a subject together with a meal or snack. In certain embodiments, a disclosed invertase and/or isomaltase enzyme or composition is administered to a subject together with each meal or snack that the subject eats. In certain embodiments, a disclosed invertase and/or isomaltase enzyme or composition is administered to a subject once every 7 days, once every 6 days, once every 5 days, once every 4 days, once every 3 days, once every 2 days, once every day, 2 times every day, 3 times every day, 4 times every day, 5 times every day, 6 times every, or more than 6 times every day.

Depending upon the circumstances, the composition can be formulated as a powder, granulate, pellet, micropellet, or a minitablet. The composition can be encapsulated in a capsule, e.g., a hydroxypropyl methylcellulose (HPMC) capsule, soft gelatin capsule, or a hard gelatin capsule. Alternatively, the composition can be formulated as a tablet dosage form. The dosage form, e.g., granules, tablets, minitablets, pellets, micropellets, capsules can be enterically coated using, e.g., one or more methacryilic polymers, such as methacrylic acid methylmethacrylate copolymers (e.g., Eudragit L and S), and methacrylic acid ethyl acrylate copolymer (Eudragit L30D), hydroxyl propyl methyl cellulose acetate, hydroxy propyl methyl cellulose phthalate succinate and others

In certain embodiments, the composition comprises less than 50%, less than 40%, less than 30%, less than 20%, less than 10% or less than 5% glycerol. In certain embodiments, the composition does not comprise glycerol.

In certain embodiments, a disclosed pharmaceutical composition has a shelf-life (e.g., retains at least 30%, at least 40%, at least 50%, at least 60%, at least 70%, at least 80%, at least 85%, at least 90%, at least 95%, at least 96%, at least 97%, at least 98%, at least 99%, or 100% of its biological activity) at room temperature of at least 3 months, 6 months, 9 months, 12 months, 15 months, 18 months, 21 months, 24 months, 36 months, 48 months, 60 months, 72 months, 84 months, 96 months, 108 months, or 120 months. In certain embodiments, a disclosed pharmaceutical composition has a shelf-life at room temperature of from about 3 to about 120 months, from about 3 to about 96 months, from about 3 to about 72 months, from about 3 to about 48 months, from about 3 to about 24 months, from about 3 to about 21 months, from about 3 to about 18 months, from about 3 to about 15 months, from about 3 to about 12 months, from about 3 to about 9 months, from about 3 to about 6 months, from about 6 to about 120 months, from about 6 to about 96 months, from about 6 to about 72 months, from about 6 to about 48 months, from about 6 to about 24 months, from about 6 to about 21 months, from about 6 to about 18 months, from about 6 to about 15 months, from about 6 to about 12 months, from about 6 to about 9 months, from about 9 to about 120 months, from about 9 to about 96 months, from about 9 to about 72 months, from about 9 to about 48 months, from about 9 to about 24 months, from about 9 to about 21 months, from about 9 to about 18 months, from about 9 to about 15 months, from about 9 to about 12 months, from about 12 to about 120 months, from about 12 to about 96 months, from about 12 to about 72 months, from about 12 to about 48 months, from about 12 to about 24 months, from about 12 to about 21 months, from about 12 to about 18 months, from about 12 to about 15 months, from about 15 to about 120 months, from about 15 to about 96 months, from about 15 to about 72 months, from about 15 to about 48 months, from about 15 to about 24 months, from about 15 to about 21 months, from about 15 to about 18 months, from about 18 to about 120 months, from about 18 to about 96 months, from about 18 to about 72 months, from about 18 to about 48 months, from about 18 to about 24 months, from about 18 to about 21 months, from about 21 to about 120 months, from about 21 to about 96 months, from about 21 to about 72 months, from about 21 to about 48 months, from about 21 to about 24 months, from about 24 to about 120 months, from about 24 to about 96 months, from about 24 to about 72 months, from about 24 to about 48 months, from about 48 to about 120 months, from about 48 to about 96 months, from about 48 to about 72 months, from about 72 to about 120 months, from about 72 to about 96 months, or from about 96 to about 120 months. In certain embodiments, a disclosed pharmaceutical composition retains at least 30%, at least 40%, at least 50%, at least 60%, at least 70%, at least 80%, at least 85%, at least 90%, at least 95%, at least 96%, at least 97%, at least 98%, at least 99%, or 100% of its biological activity after 12 hours, 1 day, 2 days, 3 days, 4 days, 5 days, 6 days, 1 week, 2 weeks, 3 weeks, or 4 weeks at 40° C. and 75% relative humidity (RH).

### V. Therapeutic Uses

The methods and compositions disclosed herein can be used to treat diseases or disorders. For example, the disclosure provides a method of treating congenital sucrase-isomaltase deficiency (CSID) in a subject. The method comprises administering (e.g., orally administering) to the subject an effective amount of (i) an invertase enzyme (e.g., a recombinant mutant invertase enzyme described herein), and/or (ii) an isomaltase enzyme. For example, the method may comprise administering a disclosed pharmaceutical composition comprising (i) a spray-dried invertase enzyme (e.g., a recombinant mutant invertase enzyme described herein), and/or (ii) a spray-dried isomaltase enzyme. In certain embodiments, the pharmaceutical composition is administered to the subject together with a meal or snack.

Under certain conditions, the method reduces abdominal pain, bloating, and/or nausea in the subject, and as a result, the method reduces a total symptom score (for example, measured by abdominal pain, bloating and nausea) for the subject. Furthermore, the invention permits greater diet liberalization in the subject (e.g., allows the subject to better tolerate certain foods, e.g., grains (e.g., wheat, potatoes, corn, rice, and bread), fruits (e.g., apples, bananas, apricots, and oranges) and/or vegetables (e.g., beets, carrots, and black beans)).

The term “effective amount” as used herein refers to the amount of an active agent (e.g., a disclosed invertase and/or isomaltase enzyme) sufficient to effect beneficial or desired results. An effective amount can be administered in one or more administrations, applications or dosages and is not intended to be limited to a particular formulation or administration route.

As used herein, “treat”, “treating” and “treatment” mean the treatment of a disease in a subject, e.g., in a human. This includes: (a) inhibiting the disease, i.e., arresting its development; and (b) relieving the disease, i.e., causing regression of the disease state. As used herein, the terms “subject” and “patient” refer to an organism to be treated by the methods and compositions described herein. Such organisms preferably include, but are not limited to, mammals (e.g., murines, simians, equines, bovines, porcines, canines, felines, and the like), and more preferably includes humans.

The disclosure also provides a method of reducing an amount of sucrose and branched (1-6 linked) α-limit dextrin in a subject, for example, a subject with congenital sucrase-isomaltase deficiency (CSID). The method comprises administering (e.g., orally administering) to the subject an effective amount of (i) an invertase enzyme (e.g., a recombinant mutant invertase enzyme described herein), and/or (ii) an isomaltase. For example, the method may comprise administering a disclosed pharmaceutical composition comprising (i) a spray-dried invertase (e.g., a recombinant mutant invertase described herein), and (ii) a spray-dried isomaltase. An amount of sucrose and branched (1-6 linked) α-limit dextrin in a subject may refer to an amount of sucrose and branched (1-6 linked) α-limit dextrin measured by a hydrogen breath test, e.g., a sucrose methane hydrogen breath test or a ¹³C-sucrose breath test. An amount of sucrose and branched (1-6 linked) α-limit dextrin in a subject may refer to an amount of sucrose and branched (1-6 linked) α-limit dextrin in a body fluid (e.g., blood, plasma, serum, or urine), tissue and/or cell in the subject.

The methods and compositions described herein can be used alone or in combination with other therapeutic agents and/or modalities. The term administered “in combination,” as used herein, is understood to mean that two (or more) different treatments are delivered to the subject during the course of the subject's affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

In certain embodiments, a method or composition described herein, is administered in combination with one or more fructose degrading enzymes. Exemplary fructose degrading enzymes include glucose (xylose) isomerases.

Throughout the description, where compositions are described as having, including, or comprising specific components, or where processes and methods are described as having, including, or comprising specific steps, it is contemplated that, additionally, there are compositions of the present invention that consist essentially of, or consist of, the recited components, and that there are processes and methods according to the present invention that consist essentially of, or consist of, the recited processing steps.

In the application, where an element or component is said to be included in and/or selected from a list of recited elements or components, it should be understood that the element or component can be any one of the recited elements or components, or the element or component can be selected from a group consisting of two or more of the recited elements or components.

Further, it should be understood that elements and/or features of a composition or a method described herein can be combined in a variety of ways without departing from the spirit and scope of the present invention, whether explicit or implicit herein. For example, where reference is made to a particular compound, that compound can be used in various embodiments of compositions of the present invention and/or in methods of the present invention, unless otherwise understood from the context. In other words, within this application, embodiments have been described and depicted in a way that enables a clear and concise application to be written and drawn, but it is intended and will be appreciated that embodiments may be variously combined or separated without parting from the present teachings and invention(s). For example, it will be appreciated that all features described and depicted herein can be applicable to all aspects of the invention(s) described and depicted herein.

It should be understood that the expression “at least one of” includes individually each of the recited objects after the expression and the various combinations of two or more of the recited objects unless otherwise understood from the context and use. The expression “and/or” in connection with three or more recited objects should be understood to have the same meaning unless otherwise understood from the context.

The use of the term “include,” “includes,” “including,” “have,” “has,” “having,” “contain,” “contains,” or “containing,” including grammatical equivalents thereof, should be understood generally as open-ended and non-limiting, for example, not excluding additional unrecited elements or steps, unless otherwise specifically stated or understood from the context.

Where the use of the term “about” is before a quantitative value, the present invention also includes the specific quantitative value itself, unless specifically stated otherwise. As used herein, the term “about” refers to a ±10% variation from the nominal value unless otherwise indicated or inferred.

It should be understood that the order of steps or order for performing certain actions is immaterial so long as the present invention remain operable. Moreover, two or more steps or actions may be conducted simultaneously.

The use of any and all examples, or exemplary language herein, for example, “such as” or “including,” is intended merely to illustrate better the present invention and does not pose a limitation on the scope of the invention unless claimed. No language in the specification should be construed as indicating any non-claimed element as essential to the practice of the present invention.

### EXAMPLES

The following Examples are merely illustrative and are not intended to limit the scope or content of the invention in any way.

### Example 1—Invertase Selection and Engineering

### Invertase Expression

This Example describes the design of recombinant mutant S. cerevisiae invertases with improved tolerance of low pH, resistance to digestion by pepsin and pancreatin, improved thermostability, and high catalytic activity.

Expression constructs encoding recombinant S. cerevisiae invertase (GenBank: AFN08663.1, SEQ ID NO: 1) were constructed for expression in the yeast expression strains Pichia pastoris and S. cerevisiae. Expression constructs were designed with or without a C-terminal or N-terminal 6×-His affinity tag and were tested for expression and tolerance of the added His tag. Further expression constructs were designed with either the natural invertase secretion leader intact (SEQ ID NO: 1) or with a substituted alpha-mating factor (FAKS) leader (SEQ ID NO: 2). The nucleic acid sequences of each expression construct were optimized for high expression in the corresponding host. The synthesized nucleic acid sequences encoding for the invertases were cloned into pD1204 (S. cerevisiae) or pD902 (P. pastoris).

For expression in S. cerevisiae, the strain CEN.PK2-1C was transformed chemically using the lithium acetate method (Ito et al. (1983) J. BACTERIOL. 153 (1): 163-168). Cells harboring plasmid were selected by plating on CM-URA media (MP Biomedicals, Santa Ana, CA) over 3 days at 30° C. Three individual colonies for each construct were isolated and used to inoculate 500 μL of CM-URA media. The inoculated culture was incubated at 30° C. with shaking at 1,000 rpm for 16 hours. 40 μL of overnight culture was then used to inoculate 500 μL of YP induction media (1% yeast extract, 2% peptone, 0.1 M potassium phosphate, 2% raffinose, and 2% galactose). The induction culture was then incubated at 30° C. with shaking at 1,000 rpm for 72 hours. The induction culture was supplemented with 50 μL 20% galactose every 24 hours after induction. The supernatant was clarified by centrifugation and secreted invertase expression was analyzed by PAGE (4-12% Bis-tris, MOPS-buffered; Invitrogen).

For expression in P. pastoris, 20 μg of expression plasmid was linearized by digestion with Pmel. The DNA was isolated by precipitation with ethanol and reconstituted in Qiagen EB buffer. For expression studies, the strain BG10 was transformed by electroporation. For protein engineering, the strain BG10 was transformed chemically using the lithium acetate method (Ito et al., supra). Cells harboring plasmid were selected by plating on YPD media with 250 μg/mL zeocin (Teknova) over 3 days at 30° C. Three individual colonies for each construct were isolated and used to inoculate 350 μl of BYPG media with 250 μg/mL zeocin in a deep-well microplate. The inoculated culture was then incubated at 30° C. with shaking at 1,000 rpm for 60 hours. 250 μL of the overnight culture was then added to 250 μL BYPM induction media (BYP media supplemented with 20% methanol). The induction culture was then incubated at 30° C. with shaking at 1,000 rpm for 72 hours. Cultures were supplemented with 50 μL BYP with 10% methanol every 12 hours after induction. Supernatants were clarified from cells by centrifugation and secreted invertase expression was analyzed by PAGE.

Expression yields were determined by PAGE densitometry after deglycosylation with PNGaseF. Briefly, culture supernatant samples were treated with PNGaseF after denaturation according to manufacturer's protocol (NEB) and the samples were analyzed by PAGE. Full-length deglycosylated invertase band intensities were determined and intensity relative to BSA standards was used to estimate protein concentration.

Expression and yield of S. cerevisiae invertase in P. pastoris was typically better than in S. cerevisiae.

### Invertase Activity and Stability Assays

In the initial stages, when the resulting proteins tested without purification, specific activities were approximated by assaying units per amount of deglycosylated protein (1 unit of enzyme consumes 1 μmole sucrose per minute at 37° C.).

Activity assays were performed using the dinitro salicylate (DNS) colorimetric method to monitor reducing sugar formation upon hydrolysis of sucrose. Briefly, the DNS method detects the reducing sugars liberated by the action of hydrolase enzymes on carbohydrates, under specific pH and temperatures (Bailey (1988) APPLIED MICROBIOLOGY AND BIOTECHNOLOGY 29:494-496). Upon reaction with a reducing sugar DNS is reduced to 3-amino-5-nitro salicylic acid and the reaction product can be monitored at 540 nm.

Culture supernatants were diluted 1/20 into reaction buffer containing 237 mM NaCl and either: A) 50 mM glycine, pH 3.5 (buffer A); B) 50 mM sodium acetate, pH 4.7 (buffer B); C) 50 mM MES, pH 5.8 (buffer C); or D) 50 mM sodium phosphate, pH 6.8 (buffer D). Diluted enzymes were then further diluted 10-fold into 10 mM sucrose in reaction buffer pre-equilibrated at 37° C. Reaction progress was then estimated by the DNS method. To stop the reaction, 20 μL of reaction was removed to 30 μL DNS reagent (10 mg/mL dinitrosalicylic acid, 300 mg/mL tartrate, 0.4 N NaOH) and the mixture was immediately heated to 99° C. for 5 minutes. 40 μL of the DNS reaction was diluted with 160 μL water and the absorbance was read at 540 nm. Reducing sugars present were determined relative to 1:1 glucose:fructose standards.

Thermostability was assayed by treatment of invertase samples at 56.5° C. for 30 minutes prior to assaying residual activity at pH 4.7 (in reaction buffer B, described above) using the DNS method as described above.

Low pH tolerance assays were performed as follows. Culture supernatants including the invertase enzymes were diluted 1/20 into 237 mM NaCl, 50 mM glycine pH 2.8 and preincubated at 37° C. for 30 minutes. Preincubations were then diluted into 10 mM sucrose in reaction buffer at a final of pH 4.7 and activity was assayed using the DNS method as described above.

Resistance to pepsin was assayed by treating the invertase enzymes with 10 mg/ml soluble pepsin from porcine gastric mucosa (Sigma-Aldrich, St. Louis, MO) in 237 mM NaCl, 50 mM NaOAc pH 4 at 37° C. Culture supernatants including the invertase enzymes were diluted 20-fold into 10 mg/mL pepsin and incubated for 2 hours at 37° C. Residual invertase activity was then assayed by diluting the pepsin treatment reaction 20-fold into 10 mM sucrose in reaction buffer at a final pH of 4.7 and activity was assayed using the DNS method as described above. The overexpressed S. cerevisiae invertase and most invertase variants showed insignificant degradation under these conditions.

Resistance to pancreatin was assayed by treating the invertases with 3.4 mg/mL soluble porcine pancreatin (Sigma-Aldrich, St. Louis, MO) in 237 mM NaCl, 50 mM MES pH 6 at 37° C. Culture supernatants were diluted 20-fold into 3.4 mg/mL pancreatin and incubated for 2 hours at 37° C. Residual invertase activity was then assayed by diluting the pancreatin treatment reaction 20-fold into 10 mM sucrose in reaction buffer at a final pH of 4.7 and activity was assayed using the DNS method as described above.

### Invertase Engineering

95 recombinant S. cerevisiae invertase variants (denoted as V1-V95) were created using protein engineering principles, each containing three substitutions relative to the wild-type natural protein S. cerevisiae invertase (denoted as V0). TABLE 2 shows certain exemplary amino acid mutations in S. cerevisiae invertase (SEQ ID NO: 2) that were analyzed in the invertase engineering program. The variants along with the wild-type (wt) S. cerevisiae invertase were expressed in P. pastoris and tested as culture supernatants for activity and stability. Each variant was tested for seven properties using the assays described above, including: 1) expression level, 2) activity at pH 3.5, 3) activity at pH 7, 4) thermostability, 5) stability at pH 2.8, 6) resistance to pepsin, and 7) resistance to pancreatin.

TABLE 2

| | | | |
|---|---|---|---|
| E120A | K277T | N429A | K519R |
| D122E | K259L | L459F | S537D |
| A123G | L359F | T470S | L549I |
| T140L | T365S | T573S | V581I |
| T140Q | A378T | S476N | G586S |
| F143Y | E382Q | S476A | N589K |
| E156Q | N390D | P479A | F596I |
| F172Y | E415S | N512G | Q597E |
| V178I | L416Q | K514P | |

Overall, 94 invertase variants were expressed and tested (one variant did not express) and compared against wild-type enzyme. In general, 13 invertase variants showed an improvement pH 2.8 stability, thermostability, pH 3.5 stability, and pH 7.0 activity over the parent invertase, 35 invertase displayed mixed properties, and 46 invertase variants displayed worse properties than the parent invertase. Exemplary results for the engineered invertase variants compared to parental, wild-type invertase (S. cerevisiae invertase) are shown in TABLE 3 and the substitutions present in the various invertase variants (relative to S. cerevisiae invertase (SEQ ID NO: 2) listed in TABLE 3 are shown in TABLE 4. TABLE 3 includes the properties of the 13 invertase variants (V6, V9, V14, V25, V32, V38, V60, V63, V70, V78, V83, V87, V89, V90), that showed one or more improved properties relative to parental invertase, and three exemplary invertase variants (V3, V75 and V82) that performed worse than the parental invertase.

TABLE 3

| | Stability at | Thermo- | Activity at | Activity at |
|---|---|---|---|---|
| Invertase | pH 2.8 ^{a)} | stability ^{a)} | pH 3.5 ^{a)} | pH 7.0 ^{a)} |
| Parental | ~ | ~ | ~ | ~ |
| invertase (V0) | | | | |
| V6 | + | ++ | ~ | + |
| V9 | ++ | + | ~ | ~ |
| V14 | + | ++ | ~ | + |
| V22 | + | − | − | − |
| V25 | ~ | ~ | ~ | + |
| V32 | + | + | + | ~ |
| V38 | ~ | + | ~ | + |
| V60 | + | + | ~ | ~ |
| V63 | ~ | + | ~ | ~ |
| V70 | ~ | ++ | ~ | + |
| V78 | + | ++ | + | ++ |
| V83 | + | ~ | ~ | ~ |
| V87 | + | + | ~ | ~ |
| V89 | + | ~ | ~ | + |
| V90 | + | + | ~ | ~ |
| V3 | − | − | − | + |
| V75 | − | − | − | − |
| V82 | − | − | − | − |

^{a)} Relative impacts of the substitutions are indicated as strongly positive (++), moderately positive (+), moderately negative (−) or neutral (~) relative to the parent invertase.

TABLE 4

| | Substitution | Substitution | Substitution | |
|---|---|---|---|---|
| Name | 1 | 2 | 3 | SEQ ID NO: |
| V6 | E120A | D122E | P479A | SEQ ID NO: 4 |
| V9 | T140L | E156Q | L549I | SEQ ID NO: 5 |
| V14 | S476A | P479A | Q597E | SEQ ID NO: 6 |
| V22 | K259L | S476N | K514P | SEQ ID NO: 7 |
| V25 | K277T | E415S | V581I | SEQ ID NO: 8 |
| V32 | A123G | T140L | F143Y | SEQ ID NO: 9 |
| V38 | N390D | S476N | K519R | SEQ ID NO: 10 |
| V60 | E382Q | T573S | V581I | SEQ ID NO: 11 |
| V63 | D122E | T470S | S476N | SEQ ID NO: 12 |
| V70 | T140Q | P479A | K514P | SEQ ID NO: 13 |
| V78 | F172Y | N429A | P479A | SEQ ID NO: 14 |
| V83 | E120A | V178I | L549I | SEQ ID NO: 15 |
| V87 | E382Q | S537D | N589K | SEQ ID NO: 16 |
| V89 | T140L | G586S | F596I | SEQ ID NO: 17 |
| V90 | E382Q | L459F | K519R | SEQ ID NO: 18 |
| V3 | K277T | T365S | N512G | |
| V75 | D122E | L359F | A378T | |
| V82 | E120A | L416Q | S537D | |

The relative impact of each substitution on each target property was estimated and is set forth in TABLE 5 (amino acid numbering is relative to S. cerevisiae invertase (SEQ ID NO: 2). In particular, the impact of certain amino acid substitutions relative to wild type S. cerevisiae invertase (SEQ ID NO: 2) that showed significant contribution to at least one of three key invertase properties: pH 7.0 activity, thermostability, or low pH survival, are summarized in TABLE 5.

TABLE 5

| Amino Acid | Activity at | Low pH | |
|---|---|---|---|
| Substitution | pH 7.0 ^{a)} | Stability ^{a)} | Thermostability ^{a)} |
| A378T | + | − − | − |
| A501N | ~ | ~ | + |
| D272N | + | ~ | ~ |
| E156Q | ~ | + | |
| E285H | ~ | ~ | ++ |
| E382Q | ~ | + | + |
| F172Y | + | + | ~ |
| G431R | ~ | + | − |
| G586S | + | ~ | ~ |
| K259L | − | ++ | ~ |
| K277T | + | ~ | − |
| L549I | + | ~ | + |
| N589K | ~ | + | + |
| P479A | ++ | + | ++ |
| T140L | ~ | + | ++ |
| V178I | ~ | + | − |
| V399A | + | − | + |
| V477D | + | ~ | ~ |
| V581I | ~ | + | + |
| D122E | − | + | + |

^{a)} Relative impacts of substitutions are indicated as strongly positive (++), moderately positive (+), moderately negative (−) or neutral (~).

### Further Characterization of Certain Invertase Variants

A number of invertase variants showing improved properties relative to the parental invertase in terms of activity and/or stability were further characterized. In particular, two studies were performed on independent preparations of the selected variants and the results are summarized in TABLES 6 and 7. TABLE 6 summarizes the characteristics of three variants compared against wild-type parental invertase.

TABLE 6

| | Activity (U/mg | | | | Stability | | | |
|---|---|---|---|---|---|---|---|---|
| | deglycosylated protein)^{a} | | | | | Thermo- | | |
| Invertase | pH | pH | pH | pH | pH | stability^{c} | | |
| Variant | 3.5 | 5.0 | 6.2 | 7.1 | 2.5^{b} | (Tm, ° C.) | Pepsin^{d} | Pancreatin^{d} |
| V0 | 768 | 956 | 660 | 171 | 16 | 55 | >80 | >80 |
| (Parent) | | | | | | | | |
| V78 | 1288 | 1520 | 1099 | 311 | 63 | 62 | >80 | >80 |
| V22 | 770 | 1029 | 637 | 96 | 76 | 54 | >80 | >80 |
| V6 | 1037 | 1188 | 830 | 247 | 60 | 61 | >80 | >80 |

^{a}Specific activity was estimated as μmole sucrose consumed per minute per mg of deglycosylated invertase in reactions (single point measurements from single variant preparations for each reaction pH).
^{b}Stability at pH 2.5 was measured as the percent residual activity remaining after pretreatment at 37° C. for 30 minutes.
^{c}Thermostability was estimated as the observed temperature of 50% loss of activity after 30 minutes of pretreatment.
^{d}Percent residual activity after protease pretreatment. Protease stability was assessed at 37° C. at 10 mg/mL pepsin in SGF, pH 4 or 3.4 mg/mL pancreatin in SIF, pH 6. All variants showed <20% loss of activity under the assay conditions after 2 hours pretreatment.

Strong improvements in activity, low pH stability and thermostability were seen for variants V78 (F172Y, N429A, P479A; SEQ ID NO: 14) and V6 (E120A, D122E, P479A; SEQ ID NO: 4). Both variants V6 and V78 contained the broadly beneficial substitution P479A and at least one other substitution positive for one or more critical properties. V22 (K259L, S476N, K514P; SEQ ID NO: 7) showed an improvement in low pH stability. V22 contained the K259L substitution which was found to be beneficial for low pH stability but not high pH activity.

To further compare enzymes that were expressed under similar conditions, Sc_S288C (SEQ ID NO: 36) invertase was expressed in P. pastoris and assayed under identical conditions as certain of the better variants. Further variants of Sc_S288C invertase were tested that included the two additional substitutions found in V78 (F172Y and N429A) to assess their impact in the context of the Sc_S288C invertase sequence. The results of the comparison are summarized in TABLE 7.

TABLE 7

| | Activity (U/mg | | | | Stability | | | |
|---|---|---|---|---|---|---|---|---|
| | deglycosylated protein)^{a} | | | | | Thermo- | | |
| Variant | pH 3.5 | pH 5 | pH 6 | pH 7 | pH 2.7^{b} | stability^{c} | Pepsin^{d} | Pancreatin^{d} |
| V0 | 960 | 1020 | 800 | 300 | 28 | 55 | >80 | >80 |
| (Parent) | | | | | | | | |
| V78 | 1540 | 1670 | 1470 | 820 | 82 | 62 | >80 | >80 |
| ScS288C | 1230 | 1330 | 1107 | 580 | 89 | 64 | nd | nd |
| ScS288C + | 1240 | 1370 | 1190 | 690 | 97 | 65 | nd | nd |
| F172Y + | | | | | | | | |
| 429A | | | | | | | | |

^{a}Specific activity was estimated as μmole sucrose consumed per minute per mg of deglycosylated invertase in reactions. Expression and reactions were performed in triplicate.
^{b}Stability at pH 2.7 was measured as the percent residual activity remaining after pretreatment at 37° C. for 30 minutes.
^{c}Thermostability was estimated as the observed temperature of 50% loss of activity after 30 minutes of pretreatment.
^{d}Percent residual activity after protease pretreatment. Protease stability was assessed at 37° C. at 10 mg/mL pepsin in SGF, pH 4 or 3.4 mg/mL pancreatin in SIF, pH 6. All variants showed <20% loss of activity under the assay conditions after 2 hours pretreatment.

The activity profile for the Sc_S288C invertase was more comparable to that of the parent S. cerevisiae invertase, but similar differences were observed in low pH and thermostability. This was consistent with the expected impact of the P479A substitution on invertase activity. The variant V78 showed improvement in activity relative to Sc_S288C invertase but slightly poorer thermostability.

Together, these studies identified several substitutions that can be used alone or in combination to positively impact activity, low pH stability and thermostability of S. cerevisiae invertase. Variant V78 shows all-around significant improvement relative to the parental invertase.

### INCORPORATION BY REFERENCE

The entire disclosure of each of the patent and scientific documents referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

What is claimed is:

1. A recombinant mutant S. cerevisiae invertase enzyme, wherein the invertase comprises: (i) increased activity at acidic pH (e.g., about pH 3.5, 4.0, 4.5, 5.0, 5.5, 6.0, or 6.5) relative to a corresponding wild-type invertase; (ii) increased activity at neutral pH (e.g., about pH 7.0) relative to a corresponding wild-type invertase; (iii) increased stability at acidic pH (e.g., about pH 2.5) relative to a corresponding wild-type invertase; (iv) increased thermal stability relative to a corresponding wild-type invertase, or a combination of (i), (ii), (iii) or (iv).

2. The invertase of claim 1, wherein the invertase comprises one or more amino acid substitutions selected from the group consisting of:
(a) a substitution of an F residue at a position corresponding to position 172 of SEQ ID NO: 2;
(b) a substitution of an N residue at a position corresponding to position 429 of SEQ ID NO: 2;
(c) a substitution of a P residue at a position corresponding to position 479 of SEQ ID NO: 2;
(d) a substitution of a D residue at a position corresponding to position 122 of SEQ ID NO: 2;
(e) a substitution of a T residue at a position corresponding to position 140 of SEQ ID NO: 2;
(f) a substitution of an E residue at a position corresponding to position 156 of SEQ ID NO: 2;
(g) a substitution of a V residue at a position corresponding to position 178 of SEQ ID NO: 2;
(h) a substitution of a K residue at a position corresponding to position 259 of SEQ ID NO: 2;
(i) a substitution of a D residue at a position corresponding to position 272 of SEQ ID NO: 2;
(j) a substitution of a K residue at a position corresponding to position 277 of SEQ ID NO: 2;
(k) a substitution of an E residue at a position corresponding to position 285 of SEQ ID NO: 2;
(l) a substitution of an A residue at a position corresponding to position 378 of SEQ ID NO: 2;
(m) a substitution of an E residue at a position corresponding to position 382 of SEQ ID NO: 2;
(n) a substitution of a V residue at a position corresponding to position 399 of SEQ ID NO: 2;
(o) a substitution of a G residue at a position corresponding to position 431 of SEQ ID NO: 2;
(p) a substitution of a V residue at a position corresponding to position 477 of SEQ ID NO: 2;
(q) a substitution of an A residue at a position corresponding to position 501 of SEQ ID NO: 2;
(r) a substitution of an L residue at a position corresponding to position 549 of SEQ ID NO: 2;
(s) a substitution of a V residue at a position corresponding to position 581 of SEQ ID NO: 2;
(t) a substitution of a G residue at a position corresponding to position 586 of SEQ ID NO: 2; or
(u) a substitution of an N residue at a position corresponding to position 589 of SEQ ID NO: 2;
or a combination of any of the foregoing substitutions.

3. The invertase of claim 2, wherein, in the invertase:
(a) the F residue at a position corresponding to position 172 of SEQ ID NO: 2 is substituted by Y (F172Y);
(b) the N residue at a position corresponding to position 429 of SEQ ID NO: 2 is substituted by A (N429A);
(c) the P residue at a position corresponding to position 479 of SEQ ID NO: 2 is substituted by A (P479A);
(d) the D residue at a position corresponding to position 122 of SEQ ID NO: 2 is substituted by E (D122E);
(e) the T residue at a position corresponding to position 140 of SEQ ID NO: 2 is substituted by L (T140L);
(f) the E residue at a position corresponding to position 156 of SEQ ID NO: 2 is substituted by Q (E156Q);
(g) the V residue at a position corresponding to position 178 of SEQ ID NO: 2 is substituted by I (V178I);
(h) the K residue at a position corresponding to position 259 of SEQ ID NO: 2 is substituted by L (K259L);
(i) the D residue at a position corresponding to position 272 of SEQ ID NO: 2 is substituted by N (D272N);
(j) the K residue at a position corresponding to position 277 of SEQ ID NO: 2 is substituted by T (K277T);
(k) the E residue at a position corresponding to position 285 of SEQ ID NO: 2 is substituted by H (E285H);
(l) the A residue at a position corresponding to position 378 of SEQ ID NO: 2 is substituted by T (A378T);
(m) the E residue at a position corresponding to position 382 of SEQ ID NO: 2 is substituted by Q (E382Q);
(n) the V residue at a position corresponding to position 399 of SEQ ID NO: 2 is substituted by A (V399A);
(o) the G residue at a position corresponding to position 431 of SEQ ID NO: 2 is substituted by R (G431R);
(p) the V residue at a position corresponding to position 477 of SEQ ID NO: 2 is substituted by D (V477D);
(q) the A residue at a position corresponding to position 501 of SEQ ID NO: 2 is substituted by N (A501N);
(r) the L residue at a position corresponding to position 549 of SEQ ID NO: 2 is substituted by I (L549I);
(s) the V residue at a position corresponding to position 581 of SEQ ID NO: 2 is substituted by I (V581I);
(t) the G residue at a position corresponding to position 586 of SEQ ID NO: 2 is substituted by S (G586S); or
(u) the N residue at a position corresponding to position 589 of SEQ ID NO: 2 is substituted by K (N589K);
or the invertase comprises a combination of any of the foregoing substitutions.

4. The invertase of any one of claims 1-3, wherein the invertase comprises one, two, three, four, five, six, seven, eight, nine, ten, or more than ten mutations relative to the corresponding wild-type invertase.

5. The invertase of claim 3 or 4, wherein the invertase comprises:
(a) the F172Y, N429A, and P479A substitutions;
(b) the E120A, D122E, and P479A substitutions;
(c) the K259L, S476N, and K514P substitutions;
(d) the T140L, E156Q, and L549I substitutions;
(e) the S476A, P479A, and Q597E substitutions;
(f) the K277T, E415S, and V581I substitutions;
(g) the A123G, T140L, and F143Y substitutions;
(h) the N390D, S476N, and K519R substitutions;
(i) the E382Q, T573S, and V581I substitutions;
(j) the D122E, T470S, and S476N substitutions;
(k) the T140Q, P479A, and K514P substitutions;
(l) the E120A, V178I, and L549I substitutions;
(m) the E382Q, S537D, and N589K substitutions;
(n) the T140L, G586S, and F596I substitutions; or
(o) the E382Q, L459F, and K519R substitutions.

6. The invertase of any one of claims 1-5, wherein the invertase comprises the amino acid sequence of any one of SEQ ID NOs: 29, 19-28, and 30-33, or an amino acid sequence that has at least 85%, 90%, 95%, 96%, 97%, 98%, or 99% sequence identity to any one of SEQ ID NOs: 29, 19-28, and 30-33.

7. The invertase of any one of claims 1-6, wherein the invertase comprises the amino acid sequence of SEQ ID NO: 29, or an amino acid sequence that has at least 85%, 90%, 95%, 96%, 97%, 98%, or 99% sequence identity to SEQ ID NO: 29.

8. The invertase of any one of claims 1-5, wherein the invertase comprises the amino acid sequence of any one of SEQ ID NOs: 14, 4-13, and 15-18, or an amino acid sequence that has at least 85%, 90%, 95%, 96%, 97%, 98%, or 99% sequence identity to any one of SEQ ID NOs: 14, 4-13, and 15-18.

9. A recombinant mutant S. cerevisiae invertase enzyme comprising a substitution, or combination of substitutions listed in TABLE 1 or TABLE 4.

10. The invertase of any one of claims 1-9, wherein the invertase has a specific activity at about pH 3.5 of at least 800, 900, 1,000, 1,100, 1,200, 1,300, 1,400, 1,500, 1,600, 1,700, 1,800, 1,900, or 2,000 μmol sucrose consumed per minute per milligram of deglycosylated invertase.

11. The invertase of any one of claims 1-10, wherein the invertase has at least 1 fold, 1.5 fold, 2 fold, 2.5 fold, or 3 fold higher activity at about pH 3.5, compared to a corresponding wild-type invertase.

12. The invertase of any one of claims 1-11, wherein the invertase has a specific activity at about pH 5.0 of at least 1,000, 1,100, 1,200, 1,300, 1,400, 1,500, 1,600, 1,700, 1,800, 1,900, 2,000, 2,100, or 2,200 μmol sucrose consumed per minute per milligram of deglycosylated invertase.

13. The invertase of any one of claims 1-12 wherein the invertase has at least 1 fold, 1.5 fold, 2 fold, 2.5 fold, or 3 fold higher activity at about pH 5.0, compared to a corresponding wild-type invertase.

14. The invertase of any one of claims 1-13, wherein the invertase has a specific activity at about pH 6.0 or 6.2 of at least 700, 800, 900, 1,000, 1,100, 1,200, 1,300, 1,400, 1,500, 1,600, 1,700, or 1,800 μmol sucrose consumed per minute per milligram of deglycosylated invertase.

15. The invertase of any one of claims 1-14 wherein the invertase has at least 1 fold, 1.5 fold, 2 fold, 2.5 fold, or 3 fold higher activity at about pH 6.0 or 6.2, compared to a corresponding wild-type invertase.

16. The invertase of any one of claims 1-15, wherein the invertase has a specific activity at about pH 7.0 or 7.1 of at least 200, 300, 400, 500, 600, 700, 800, 900, 1,000, 1,100, 1,200, or 1,300 μmol sucrose consumed per minute per milligram of deglycosylated invertase.

17. The invertase of any one of claims 1-16, wherein the invertase has at least 1 fold, 1.5 fold, 2 fold, 2.5 fold, or 3 fold higher activity at about pH 7.0 or 7.1, compared to a corresponding wild-type invertase.

18. The invertase of any one of claims 1-17, wherein the invertase retains at least 20%, 30%, 40%, 50%, 60%, 70%, 80%, 90%, 95%, 96%, 97%, 98%, or 99% of activity following incubation at about pH 2.5 for about 30 minutes.

19. The invertase of any one of claims 1-18, wherein the invertase has at least 1 fold, 1.5 fold, 2 fold, 2.5 fold, 3 fold, 3.5 fold, 4 fold, or 5 fold higher stability at about pH 2.5 compared to a corresponding wild-type invertase.

20. The invertase of any one of claims 1-19, wherein the invertase has a Tm of at least 56, 57, 58, 59, 60, 61, 62, 63, 64, 65, 66, 67, or 68° C.

21. The invertase of any one of claims 1-20, wherein the invertase has a Tm that is at least 1, 2, 3, 4, 5, 6, 7, 8, 9, or 10° C. higher than a corresponding wild-type invertase.

22. The invertase of any one of claims 1-21, wherein the invertase has higher stability in the presence of pancreatin or pepsin compared to a corresponding wild-type invertase.

23. A nucleic acid encoding the invertase of any one of claims 1-22.

24. An expression vector comprising the nucleic acid of claim 23.

25. A cell comprising the expression vector of claim 24.

26. The cell of claim 25, wherein the cell is a P. pastoris or S. cerevisiae cell.

27. A method of producing a recombinant mutant S. cerevisiae invertase enzyme, the method comprising
a) growing the cell of claim 25 or 26 under conditions so that the host cell expresses the invertase, and
b) purifying the invertase.

28. A pharmaceutical composition comprising the invertase of any one of claims 1-22, and a pharmaceutically acceptable carrier and/or an excipient.

29. A pharmaceutical composition comprising an invertase enzyme, a separate isomaltase enzyme, and a pharmaceutically acceptable carrier and/or an excipient.

30. The pharmaceutical composition of claim 29, wherein the invertase is spray-dried.

31. The pharmaceutical composition of claim 29 or 30, wherein the isomaltase is spray-dried.

32. The pharmaceutical composition of any one of claims 29-31, wherein the invertase is a microbial invertase, or a functional fragment or variant thereof.

33. The pharmaceutical composition of any one of claims 29-32, wherein the isomaltase is a microbial isomaltase, or a functional fragment or variant thereof.

34. The pharmaceutical composition of any one of claims 29-33, wherein the invertase is derived from Saccharomyces cerevisiae.

35. The pharmaceutical composition of claim 34, wherein the invertase comprises a sequence of any one of SEQ ID NOs: 29, 14, 1-13, 15-28, and 30-33, or a functional fragment or variant thereof.

36. The pharmaceutical composition of claim 34, wherein the invertase is the recombinant mutant S. cerevisiae invertase enzyme of any one of claims 1-19.

37. The pharmaceutical composition of any one of claims 29-36, wherein the isomaltase is derived from Saccharomyces cerevisiae.

38. The pharmaceutical composition of claim 37, wherein the isomaltase comprises any one of SEQ ID NOs: 37-41, or a functional fragment or variant thereof.

39. The pharmaceutical composition of any one of claims 29-36, wherein the isomaltase comprises a recombinant mutant Lactobacillus fermentum isomaltase enzyme (SEQ ID NO: 47), or a functional fragment or variant thereof.

40. The pharmaceutical composition of claim 39, wherein the isomaltase further comprises:
(a) K115I, R132K, D226S, E310A, L366M, I421A, A444G, A531D, and F560V substitutions;
(b) E93K, K115I, R132K, D226S, E310A, L366M, I421A, A444G, A531D, and F560L substitutions;
(c) E93K, K115I, R132K, D226S, E310A, I421A, A444G, E524Q, A531D, and F560L substitutions;
(d) E93K, K115I, R132K, D226S, E310A, I421A, A444G, A531D, and F560V substitutions;
(e) E93K, K115I, R132K, D226S, L366M, I421A, A444G, E524Q, A531D, and F560L substitutions;
(f) E93K, K115I, R132K, D226S, L366M, I421A, A444G, A531D, and F560V substitutions;
(g) E93K, K115I, R132K, D226S, I421A, A444G, E524Q, A531D, and F560V substitutions;
(h) K115I, R132K, D226S, E310A, L366M, I421A, A444G, E524Q, A531D, and F560L substitutions;
(i) K115I, R132K, D226S, E310A, I421A, A444G, E524Q, A531D, and F560V substitutions;
(j) K115I, R132K, D226S, L366M, 1421A, A444G, E524Q, A531D, and F560V substitutions;
(k) E93K, K115I, R132K, A211E, D226S, I421A, A444G, E524Q, A531D, and F560V substitutions;
(l) E93K, K115I, R132K, D226S, L366M, I421A, A444G, E524Q, A531D, and F560V substitutions;
(m) E93K, K115I, R132K, D226S, E275D, I421A, A444G, E524Q, A531D, and F560V substitutions;
(n) T89A, E93K, K115I, R132K, D226S, I421A, A444G, E524Q, A531D, and F560V substitutions;
(o) E93K, K115I, R132K, H182R, D226S, I421A, A444G, E524Q, A531D, and F560V substitutions;
(p) E93K, K115I, E122D, R132K, D226S, I421A, A444G, E524Q, A531D, and F560V substitutions;
(q) E93K, K115I, D226S, I421A, A444G, E524Q, A531D, and F560V substitutions;
(r) E93K, K115I, D226S, I421A, A444G, E524Q, A531D, and F560L substitutions;
(s) K115I, A211E, D226S, I421A, A444G, A531D, and F560V substitutions;
(t) K115I, D226S, L366M, I421A, A444G, E524Q, A531D, and F560L substitutions;
(u) K115I, A211E, D226S, I421A, A444G, E524Q, A531D, and F560L substitutions;
(v) K115I, R132K, D226S, E310A, L366M, 1421A, A444G, A531D, and F560L substitutions;
(w) E93K, K115I, D226S, E310A, I421A, A444G, A531D, and F560L substitutions;
(x) K115I, D226S, E310A, I421A, A444G, A531D, and F560V substitutions;
(y) E93K, K115I, D226S, L366M, 1421A, A444G, A531D, and F560L substitutions;
(z) K115I, D226S, I421A, A444G, E524Q, A531D, and F560V substitutions;
(aa) K115I, D226S, E310A, I421A, A444G, E524Q, A531D, and F560L substitutions;
(bb) K115I, A211E, D226S, L366M, I421A, A444G, A531D, and F560L substitutions;
(cc) K115I, A211E, D226S, E310A, I421A, A444G, A531D, and F560L substitutions;
(dd) E93K, K115I, D226S, 1421A, A444G, A531D, and F560V substitutions;
(ee) E93K, K115I, A211E, D226S, I421A, A444G, A531D, and F560L substitutions; or
(ff) K115I, D226S, L366M, I421A, A444G, A531D, and F560V substitutions.

41. The pharmaceutical composition of claim 39 or 40, wherein the invertase comprises the amino acid sequence of SEQ ID NO: 14 or 29, or a functional fragment thereof.

42. The pharmaceutical composition of claim 39 or 40, wherein the invertase comprises the amino acid sequence of SEQ ID NO: 49 or 50, or a functional fragment thereof.

43. The pharmaceutical composition of any one of claims 28-42, wherein the composition is formulated as an oral dosage form.

44. The pharmaceutical composition of claim 43, wherein the composition is a formulated as a powder, satchel, granulate, pellet, micropellet, tablet, or minitablet.

45. The pharmaceutical composition of claim 44, wherein the composition is formulated as a powder, satchel, or tablet.

46. The pharmaceutical composition of any one of claims 28-45, wherein the composition has a shelf-life at room temperature of at least 3 months, 6 months, 9 months, 12 months, 15 months, 18 months, 21 months, 24 months, 36 months, 48 months, 60 months, 72 months, 84 months, 96 months, 108 months, or 120 months.

47. A method of treating congenital sucrase-isomaltase deficiency (CSID) in a subject in need thereof, the method comprising orally administering to the subject and effective amount of the pharmaceutical composition of any one of claims 28-46.

48. The method of claim 47, wherein the pharmaceutical composition is administered to the subject together with a meal or snack.

49. A method of reducing sucrose and branched (1-6 linked) α-limit dextrin concentration in a subject, the method comprising orally administering to the subject and effective amount of the pharmaceutical composition of any one of claims 28-46.

50. The method of claim 49, wherein the method reduces sucrose and branched (1-6 linked) α-limit dextrin concentration in the subject as measured by a hydrogen breath test.

51. A method of treating congenital sucrase-isomaltase deficiency (CSID) in a subject in need thereof, the method comprising orally administering to the subject (i) an invertase enzyme and (ii) a separate isomaltase enzyme.

52. The method of claim 51, wherein the invertase is the recombinant mutant S. cerevisiae invertase enzyme of any one of claims 1-22.

53. The method of claim 52, wherein the isomaltase is the recombinant mutant S. cerevisiae or L. fermentum isomaltase enzyme of any one of claims 37-40.

54. The method of any one of claims 47-53, wherein the subject is a mammal.

55. The method of any one of claims 47-53, wherein the subject is a human.
